(19) United States
(12) Patent Application Publication
(10) Pub. No.: US 2004/0033517 A1
(43) Pub. Date: Feb. 19, 2004

(54) Compositions and methods relating to endothelial cell signaling using the protease activated receptor (PAR1)

(76) Inventors: Wolfram Ruf, San Diego, CA (US); Matthias Riewald, La Jolla, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/418,938

(22) Filed: Apr. 18, 2003

Related U.S. Application Data
(60) Provisional application No. 60/374,110, filed on Apr. 19, 2002.

(51) Int. Cl. C12Q 1/68; G01N 33/53; G01N 33/567

(52) U.S. Cl. 435/6; 435/7.2

(57) ABSTRACT

The invention relates to compositions and methods based on the characterization of an endothelial cell protein C receptor (EPCR) dependent signaling by activated protein C (APC) which acts through protease activated receptor 1 (PAR1).

### CROSS-REFERENCE TO RELATED APPLICATIONS

This application claims benefit of U.S. Provisional Application No. 60/374,110, filed Apr. 19, 2002, the disclosure of which is incorporated herein by reference.

### FIELD OF THE INVENTION

The invention relates to compositions, methods of identifying molecules, and methods of screening based on the characterization of an endothelial cell protein C receptor (EPCR) dependent signaling by activated protein C (APC) which acts through protease activated receptor 1 (PAR1). The invention further provides methods of treatment in mammalian subjects for various disease states such as inflammation or sepsis.

### BACKGROUND OF THE INVENTION

Tissue factor initiated coagulation in sepsis triggers a lethal response (A. A. Creasey et al., J Clin Invest 91: 2850, 1993; F. B. Taylor et al., Blood 91: 1609, 1998; F. B. Taylor, Jr. et al., Circ Shock 33: 127, 1991) that may involve coagulation protease mediated proinflammatory signaling through G-protein coupled PARs (S. R. Coughlin, Nature 407: 258, 2000; J. O'Brien, et. al., Oncogene 20: 1570, 2001; Camerer, et al., Proc Natl Acad Sci USA 97: 5255, 2000; Riewald, W. Ruf, Proc Natl Acad Sci USA 98: 7742, 2001). The PC pathway protects animals from E. coli induced lethality (F. B. Taylor et al., Blood 95: 1680, 2000; F. B. Taylor, Jr., in Bacterial Endotoxins: Basic Science to Anti-Sepsis Strategies, J. Levin, S. J. H. Van Deventer, T. Van der Poll, A. Sturk, Eds. (Wiley-Liss, Inc., New York, 1994); F. B. Taylor et al., J Clin Invest 79: 918, 1987) and APC reduces mortality in patients with severe sepsis (G. R. Bernard et al., N Engl J Med 344: 699, 2001). PC bound to EPCR is activated by a coagulation feedback loop in which traces of thrombin, once bound to thrombomodulin, specifically activate PC(C. T. Esmon, Faseb J 9: 946, 1995). APC is a trypsin-like coagulation protease and PARs serve as the cellular sensors for these enzymes (S. R. Coughlin, Nature 407: 258, 2000; J. O'Brien, et. al., Oncogene 20: 1570, 2001). The PC anticoagulant pathway operates on endothelial cells that express PAR1 and PAR2 along with EPCR. Proteolytic signaling by APC induces protective responses in endothelial cells (D. E. Joyce, et al., J Biol Chem 276: 11199, 2001), but the involvement of PARs in this process remains unclear.

The events accompanying sepsis remain complex, although it is known that the coagulant and inflammatory exacerbation in sepsis is counterbalanced by the protective effects mediated by the protein C (PC) pathway. Cellular responses to injury at the level of platelets and hemostasis and inflammatory exacerbation in sepsis are mediated, in part, by thrombin activation of closely related G-protein coupled receptors, namely protease-activated receptors (PAR)-1, -3, and -4. Endothelial cell protein C receptor (EPCR) plays a crucial role in the protection from septicemia by activated protein C (APC), although the precise signaling mechanism of APC remains unclear.

### SUMMARY OF THE INVENTION

The coagulant and inflammatory exacerbation in sepsis is counterbalanced by the protective protein C (PC) pathway. Activated protein C (APC) was shown to utilize endothelial cell PC receptor (EPCR) as a coreceptor for cleavage of protease activated receptor 1 (PAR1) on endothelial cells. APC signals through PAR1, also known to be a receptor for thrombin, on endothelial cells. Specifically, APC activates PAR1 in an EPCR dependent manner, resulting in PAR1 signaling.

Gene profiling demonstrated that PAR1 signaling could account for all APC induced protective genes. The compositions and methods described herein provide examples of APC induced protective genes. For example, the immuno-modulatory monocyte chemoattractant protein-1 (MCP-1) was selectively induced by PAR1 activation, but not by PAR2 activation. Thus, the prototypical thrombin receptor is the target for EPCR-dependent APC signaling, suggesting a role for this receptor cascade in protection from sepsis.

Compositions and methods are described herein which are useful for the diagnosis and treatment of conditions related to inflammation and sepsis that are influenced by signaling through PAR1 by interactions of APC and EPCR.

The methods describe a variety of cell based assay systems for measuring signaling in endothelial cells, which signaling can depend on APC, on EPCR, on PAR1, on PAR2, or on combinations thereof. Using these systems, one can screen for compounds which modulate signaling. These identified compounds are therapeutic candidates for use in modulating specific signaling pathways, and in modulating inflammatory processes or sepsis regulated by these signaling pathways.

In one aspect, a method for identifying a compound which modulates signaling in endothelial cells via a protease activated receptor 1 (PAR1) pathway is provided which comprises contacting a test compound with a cell-based assay system comprising a cell co-expressing endothelial protein C receptor (EPCR) and PAR1 capable of signaling responsiveness to activated protein C (APC). The method provides APC to the assay system in an amount selected to be effective to activate signaling, and detects an effect of the test compound on PAR1 signaling in the assay system, effectiveness of the test compound in the assay being indicative of the modulation. In some such methods, the cell-based assay system can comprise an endothelial cell or a PAR1 deficient fibroblast cell. The method further comprises measuring a reporter gene activity in the endothelial cell or the PAR1 deficient fibroblast cell. In a further aspect of the method, the detecting step further comprises determining a level of gene expression for a panel of genes in endothelial cells subject to activation or repression mediated by APC, EPCR, and PAR1.

In a detailed aspect of the method, the panel of genes includes at least one of SH-PTP3, W28170, fructose-6-phosphate, 2-kinase/fructose-2,6-bisphosphatase, W28616, BID, NF-KB2, thrombospondin-1,6-phosphofructo-2-kinase/fructose-2,6-bisphosphatase, neurofibromatosis 2 tumor suppressor, cationic amino acid transporter 2, W28616, NF-kappa-B p65delta3, TTF-I interacting peptide 12, ATRC2, alpha adducin, sortilin, p53 cellular tumor antigen, W28257, hERRa1, G protein-coupled receptor, EGFRBP-GRB2, Pax8, SH-PTP3, 5T4, bc1-xL, disintegrin-metalloprotease, C1q-related factor, cyclin D1, AI743606, myosin-IXb, GNS, ISLR, Stat2, stem cell factor, c-ets-1, usurpin-beta, chromosome 5q21-22, clone-A3-A, thrombospondin, ELL2, dual-specificity protein phosphatase, vitronectin receptor alpha subunit, PCTAIRE-2, follistatin, ets-2, ELL2, utrophin, C8FW phosphoprotein, PCTAIRE-2, fra-2, MINOR, GADD34, IL8, SSR alpha, neuron derived orphan receptor, CGGBP, nma, jun-B, epsilon tyrosine phosphatase, GRO-beta, CtIP, PRDII-BF1, vascular endothelial growth factor, stanniocalcin-related protein, CL 100, BMP-2A, NF-ATc, mrg1, jun-B, VEGF, STC, or ATF3. In a futher detailed aspect of the method, the panel of genes includes at least one of MCP-1, qe82d12.x1, RACH1, PAC747L4, T-plastin, endothelin receptor type B-like, VEGF, or wx69d10.x1.

In another aspect, a method for identifying genes which reduce an inflammatory response in a tissue is provided which comprises contacting a test compound with a cell-based assay system comprising an endothelial cell capable of signaling responsiveness to APC by an EPCR-dependent PAR1 pathway. The method provides stimulating the cell-based assay system with a test compound specific for signaling by APC or PAR1, and isolating cDNA corresponding to mRNA in stimulated cells. The method further provides analyzing the isolated cDNA using a gene expression analysis system to identify a panel of genes that are regulated in stimulated cells by signaling by APC through the EPCR-dependent PAR1 signaling pathway, wherein regulation of one or more of the genes is capable of reducing an inflammatory response in the tissue.

In another aspect, a method for identifying genes which reduce an inflammatory response in a tissue is provided which comprises contacting a test compound with a cell-based assay system comprising an endothelial cell capable of signaling responsiveness to APC by an EPCR-dependent PAR1 pathway, and stimulating the cell-based assay system with a test compound specific for signaling by APC or PAR1. The method further comprises isolating cDNA corresponding to mRNA in stimulated cells, and analyzing the isolated cDNA using a gene expression analysis system to identify a panel of genes that are regulated in stimulated cells by signaling by APC through the EPCR-dependent PAR1 signaling pathway, wherein regulation of one or more of the genes is capable of reducing an inflammatory response in the tissue.

In a further aspect, the method comprises analyzing the isolated cDNA using the gene expression analysis system to identify the panel of genes that are up-regulated by signaling through PAR1 or APC and down-regulated by signaling through PAR2. In a further detailed aspect, the panel of genes includes at least one of MCP-1, qe82d12.x1, RACH1, PAC747L4, T-plastin, endothelin receptor type B-like, VEGF, or wx69d10.x1.

In a further aspect the method comprises analyzing the isolated cDNA using the gene expression analysis system to identify the panel of genes that are down-regulated by signaling through PAR1 or APC and up-regulated by signaling through thrombin. In a further detailed aspect, the panel of genes includes at least one of SH-PTP3, W28170, fructose-6-phosphate, 2-kinase/fructose-2,6-bisphosphatase, W28616, BID, NF-KB2, thrombospondin-1, 6-phosphofructo-2-kinase/fructose-2,6-bisphosphatase, neurofibromatosis 2 tumor suppressor, cationic amino acid transporter 2, W28616, NF-kappa-B p65delta3, TTF-I interacting peptide 12, ATRC2, alpha adducin, sortilin, p53 cellular tumor antigen, W28257, hERRa1, G protein-coupled receptor, EGFRBP-GRB2, Pax8, SH-PTP3, 5T4, bc1-xL, disintegrin-metalloprotease, C1q-related factor, cyclin D1, A1743606, myosin-IXb, GNS, ISLR, Stat2, stem cell factor, c-ets-1, usurpin-beta, chromosome 5q21-22, clone-A3-A, thrombospondin, ELL2, dual-specificity protein phosphatase, vitronectin receptor alpha subunit, PCTAIRE-2, follistatin, ets-2, ELL2, utrophin, C8FW phosphoprotein, PCTAIRE-2, fra-2, MINOR, GADD34, IL8, SSR alpha, neuron derived orphan receptor, CGGBP, nma, jun-B, epsilon tyrosine phosphatase, GRO-beta, CtIP, PRDII-BF1, vascular endothelial growth factor, stanniocalcin-related protein, CL 100, BMP-2A, NF-ATc, mrg1, jun-B, VEGF, STC, or ATF3

In another aspect, a method for identifying a compound which modulates signaling in endothelial cells via a PAR1 pathway is provided which comprises contacting a test compound with a cell-based assay system comprising an endothelial cell capable of signaling responsiveness to APC by an EPCR-dependent PAR1 pathway. The method provides stimulating the cell-based assay system with the test compound specific for signaling by APC or PAR1. The method further provides isolating cDNA corresponding to mRNA in two populations of cells stimulated in both the presence and absence of the test compound. The method further provides analyzing the isolated cDNA using a gene expression analysis system to identify a panel of genes, for example, from the genes described herein, which are regulated in stimulated cells, and detecting up-regulated or down-regulated genes in both populations of stimulated cells, thereby identifying the test compounds that modulate signaling through APC or PAR1.

In a further aspect, the detecting step of the method comprises identifying the test compounds that regulate gene expression by up-regulating genes that signal through PAR1 or APC and down-regulating genes that signal through PAR2. In a further aspect, the detecting step of the method comprises identifying the test compounds that regulate gene expression by down-regulating genes that signal through PAR1 or APC and by up-regulating genes that signal through thrombin.

In another aspect, a method for detecting an activation state of an EPCR-dependent PAR1 pathway in a tissue is provided which comprises contacting a test compound with a cell-based assay system comprising an endothelial cell capable of signaling responsiveness to APC by an EPCR-dependent PAR1 pathway. The method further provides stimulating the cell-based assay system with the test compound specific for signaling by APC or PAR1. The method further provides identifying a panel of genes which represent regulated genes that correlate with an activation state for endothelial cell signaling mediated by APC, EPCR or PAR1, and determining levels of gene expression for members of the panel of genes in the tissue subject to activation mediated by APC or PAR1 thereby determining the gene activation state of the tissue.

In a further aspect, the determining step of the method comprises identifying the levels of gene expression for members of the panel of genes that are up-regulated by signaling through APC and EPCR-dependent PAR1 pathway, and identifying the panel of genes, for example, from the genes described herein, that are down-regulated by signaling through PAR2. In a further aspect, the determining step of the method step comprises identifying the levels of gene expression for the panel of genes, for example, from the genes described herein, that are down-regulated by signaling through APC and EPCR-dependent PAR1 pathway, and identifying the levels of gene expression for the panel of genes that are up-regulated by signaling through thrombin.

In another aspect, a method for treating inflammation or sepsis in a mammalian subject is provided which comprises administering a therapeutically effective amount of a compound which modulates signaling in endothelial cells via an EPCR-dependent PAR1 pathway, wherein the compound acts as an agonist of APC-mediated PAR1 signaling in a cell-based assay system, and the compound is effective to reduce the incidence of inflammation or sepsis in the mammalian subject.

In a further aspect, the method comprises identifying genes that are up-regulated by signaling through APC, EPCR, and PAR1, and identifying genes that are down-regulated by signaling through PAR2. In a further aspect, the method comprises identifying genes that are down-regulated by signaling through APC, EPCR, or PAR1 and up-regulated by signaling through thrombin. In a detailed aspect the method further comprises administering a therapeutically effective amount of a second compound acting as an antagonist of thrombin-mediated PAR1 signaling in a cell-based assay system, the second compound is effective to reduce the incidence of inflammation or sepsis in the mammalian subject.

In another aspect, a method for treating stroke in a mammalian subject is provided which comprises administering a therapeutically effective amount of a compound which modulates signaling in endothelial cells via an EPCR-dependent PAR1 pathway, wherein the compound acts as an agonist of APC-mediated PAR1 signaling in a cell-based assay system and the compound is effective to reduce the incidence of stroke in the mammalian subject.

In a further aspect, the method comprises identifying genes that are up-regulated by signaling through APC, EPCR, and PAR1, and identifying genes that are down-regulated by signaling through PAR2. In a further aspect, the method comprises identifying genes that are down-regulated by signaling through APC, EPCR, or PAR1 and up-regulated by signaling through thrombin. In a detailed aspect the method further comprises administering a therapeutically effective amount of a second compound acting as an antagonist of thrombin-mediated PAR1 signaling in a cell-based assay system, the second compound is effective to reduce the incidence of stroke in the mammalian subject.

In another aspect, a method for treating ischemic injury in a mammalian subject is provided which comprises administering a therapeutically effective amount of a compound which modulates signaling in endothelial cells via an EPCR-dependent PAR1 pathway, wherein the compound acts as an agonist of APC-mediated PAR1 signaling in a cell-based assay system and the compound is effective to reduce the incidence of ischemic injury in the mammalian subject.

In a further aspect, the method comprises identifying genes that are up-regulated by signaling through APC, EPCR, and PAR1, and identifying genes that are down-regulated by signaling through PAR2. In a further aspect, the method comprises identifying genes that are down-regulated by signaling through APC, EPCR, or PAR1 and up-regulated by signaling through thrombin. In a detailed aspect the method further comprises administering a therapeutically effective amount of a second compound acting as an antagonist of thrombin-mediated PAR1 signaling in a cell-based assay system, the second compound is effective to reduce the incidence of ischemic injury in the mammalian subject.

In another aspect, a method for preventing apoptosis in a mammalian cell is provided which comprises administering a therapeutically effective amount of a compound which modulates signaling in endothelial cells via an EPCR-dependent PAR1 pathway, wherein the compound acts as an agonist of APC-mediated PAR1 signaling in a cell-based assay system and the compound is effective to reduce the incidence of apoptosis in the mammalian cell.

In a further aspect, the method comprises identifying genes that are up-regulated by signaling through APC, EPCR, and PAR1, and identifying genes that are down-regulated by signaling through PAR2. In a further aspect, the method comprises identifying genes that are down-regulated by signaling through APC, EPCR, or PAR1 and up-regulated by signaling through thrombin. In a detailed aspect the method further comprises administering a therapeutically effective amount of a second compound acting as an antagonist of thrombin-mediated PAR1 signaling in a cell-based assay system, the second compound is effective to reduce the incidence of apoptosis in the mammalian cell.

In another aspect, a method of identifying a test compound that prevents apoptosis in a mammalian cell is provided which comprises contacting a test compound to the mammalian cell in a cell-based assay system to co-express EPCR and PAR1 capable of signaling responsiveness to APC, and assaying for an effect of the test compound on death of the mammalian cell, thereby identifying compounds that prevent apoptosis in the mammalian cell.

In another aspect, a method of screening drug candidates in a mammalian subject is provided which comprises administering a therapeutically effective amount of a compound to the mammalian subject wherein the compound acts as an agonist of an EPCR-dependent PAR1 pathway, and wherein the compound modulates signaling via a PAR1 signaling pathway in an endothelial cell-based assay system.

In a further aspect, the method comprises administering a therapeutically effective amount of a second compound acting as an antagonist of thrombin-mediated PAR1 signaling in a cell-based assay system, the second compound modulates signaling via a PAR1 signaling pathway in an endothelial cell-based assay system. In a further aspect, the method provides signaling that down-regulates genes by APC/EPCR/PAR1 signaling and up-regulates genes by thrombin signaling. In a further aspect, the method provides signaling that up-regulates genes by APC/EPCR/PAR1 signaling and down-regulates genes by PAR2 signaling. In a detailed aspect, up-regulation by APC/EPCR/PAR1 signaling is approximately 1.3-fold or greater, and down-regulation by PAR2 is less than approximately 1.3-fold.

As further contemplated, it has now been discovered that APC signals through the thrombin receptor known as protease activated receptor 1 (PAR1) on endothelial cells. Specifically, it is discovered that APC activates PAR1 in an EPCR dependent manner, resulting in signaling. Details of the discoveries are illustrated in the exemplary embodiments.

As described herein, a variety of compositions and methods which are useful for the diagnosis and potential treatment of conditions related to inflammation and sepsis that are influenced by signaling through PAR1 by interactions of APC and EPCR.

According to the teachings, it is seen that there are a variety of cell based assay systems for measuring signaling in endothelial cells, which signaling can depend on APC, on EPCR, on PAR1, on PAR2, or on combinations thereof. Using these systems, one can screen for compounds which modulate signaling. These identified compounds are therapeutic candidates for use in modulating specific signaling pathways, and in modulating inflammatory processes or sepsis regulated by these signaling pathways.

Thus, in one embodiment, a method for screening for a compound which modulates signaling in endothelial cells via the PAR1 pathway is described comprising: a) providing a cell-based assay system comprising an endothelial cell co-expressing EPCR and PAR1 capable of signaling responsiveness to APC according to exemplary embodiments; b) admixing a test compound with the cell-based assay system in the presence or absence of an activating amount of APC; and c) determining the effect of the test compound on signaling in the cell-based assay system, thereby identifying a compound which modulates PAR1 dependent signaling.

In a related embodiment, a screening method for identifying genes that participate in the regulation of the subject signaling pathways is described. Using the methods illustrated in an exemplary embodiment, one can measure fluctuations in levels of gene expression of selected genes that regulate signaling. Similarly, gene microarray analysis can be used to identify new genes which are involved in regulation of the signaling pathways. Exemplary methods of microarray analysis are described in exemplary embodiments

Thus, a method for identifying genes which are regulated in endothelial cells is described comprising:

a) providing a cell-based assay system comprising an endothelial cell capable of signaling responsiveness to APC by an EPCR-dependent PAR1 pathway according to exemplary embodiments;

b) stimulating the cell-based assay system with a compound specific for signaling by APC, PAR1 or PAR2;

c) isolating cDNA corresponding to the mRNA in the stimulated cells;

d) analyzing the isolated cDNA using a gene expression analysis system to identify genes which are upregulated or downregulated in the stimulated cells, thereby identifying genes that are regulated by signaling through APC, PAR1 or PAR2.

Many genes have been identified by the present methods which are useful targets for detecting the activation state of a tissue (e.g., Examples 1 and 2; FIGS. 1, 2, and 3). Using a panel of these target genes, one can evaluate the status of the activation state of preselected genes in a tissue sample obtained from a patient experiencing an episode or at risk for an inflammatory condition, sepsis or other condition in which the status of regulation of signaling is diagnostic or prognostic for evaluation or treatment of the condition. Thus, the invention contemplates a method for detecting the activation state of the APC, PAR1 or PAR2 pathway in a tissue comprising: a) providing a panel of genes according to exemplary embodiments which represent regulated genes that correlate with an activation state for endothelial cell signaling mediated by EPCR, PAR1, PAR2 or APC; and b) determining the levels of gene expression for members of the panel of genes in a tissue containing endothelial cells subject to activation mediated by EPCR, PAR1, PAR2 or APC, thereby determining the gene activation state of the tissue.

These target genes can be also used in screening assays to identify compounds capable of modulating the signaling that is regulated by the target gene.

Further contemplated is a method for screening for a compound which modulates signaling in endothelial cells comprising: a) providing a cell-based assay system comprising an endothelial cell capable of signaling responsiveness to APC by an EPCR-dependent PAR1 pathway according to the exemplary embodiments; b) stimulating the cell-based assay system with a compound specific for signaling by APC, PAR1 or PAR2 in the presence or absence of a test compound; c) isolating cDNA corresponding to the mRNA in the cells stimulated in both the presence and absence of the test compound; d) analyzing the isolated cDNA using a gene expression analysis system to identify genes which are regulated in the stimulated cells; and e) comparing the up- or down-regulated genes in both populations of stimulated cells, thereby identifying compounds that are modulated by signaling through APC, PAR1 or PAR2.

Also contemplated are compositions and articles of manufacture which contain one or more of the reagents described in the exemplary embodiments formulated and/or packaged for use in practicing the methods of the invention.

### BRIEF DESCRIPTION OF THE DRAWINGS

FIG. 1. Cell activation by APC is dependent on EPCR and PAR expression. (A) Induction of egr-1 promoter activity in PAR1-deficient fibroblasts transfected with human EPCR, EPCR variants with Tyr154→Ala (EPCR Al 54) or Cys221→Ser (EPCR S221) substitutions, human PAR2 or PAR1. Fold-induction of luciferase activity upon stimulation with 20 nM APC (solid bars), PAR agonist peptide (open bars; 100 μM SLIGRL and 10 μM TFLLRN in panel A), or 5 nM thrombin (crosshatched bars) is shown (n=3). (panel B) APC signaling in HUVEC measured as Erk1/2 phosphorylation. Stimulation with the indicated agonists in the absence (filled bars) or presence of 100 nM chloromethylketone modified APC (APC-CK) (open bars, n=3) or cleavage blocking anti PAR1 antibodies (open bars, n=5), *p<0.05. A representative Western-blot is shown in panel C.

FIG. 2. APC and PAR1 specific agonist induce similar genes in human endothelial cells. (A) Plot of fold induction (average, n=3) by PAR1 versus PAR2 agonist peptides demonstrated selective upregulation of MCP-1 by the PAR1 agonist. Both MCP-1 and the nuclear receptor TR3 were represented by two independent probe sets. Comparison of genes induced by APC versus PAR1 (B) and PAR2 (C, D) agonist peptides showed upregulation of MCP-1 by APC stimulation.

FIG. 3. (A) Time course of TR3, MCP-1, and DSCR1 induction in HUVECs by PAR1 agonist (filled circles), PAR2 agonist (filled squares), and APC (open circles) was analyzed by quantitative PCR. Fold inductions, normalized to GAPDH levels, are shown for a typical experiment. (B) Fold inductions of the indicated genes following stimulation with 10 nM APC/100 nM hirudin in the absence and presence of anti PAR1 antibody were determined by quantitative PCR (n=3). HBEGF, heparin binding EGF-like growth factor; NFκBIα, NFκB Inhibitor α; GADD45B, growth arrest and DNA damage-inducible gene beta.

### DETAILED DESCRIPTION OF ILLUSTRATIVE EMBODIMENTS

The term “signaling in endothelial cells” refers to the interaction of a ligand, such as APC, with receptors, such as endothelial protein C receptor (EPCR) and protease activated receptor (PAR)-1 resulting in endothelial cell protective signaling within the protective protein C pathway or the protease activated receptor (PAR)-1 pathway.

The term “activated protein C (APC)” refers to a protease shown to use endothelial protein C receptor (EPCR) as a coreceptor for cleave of PAR1 on endothelial cells. The protective protein C pathway counterbalances the coagulant and inflammatory exacerbation in sepsis.

The term “activating amount of APC” refers to an amount of APC interacting with cellular receptors, such as PAR1 that results in expression of genes leading to levels of protective protein activity in endothelial cells.

The term “endothelial protein C receptor (EPCR)” refers to a receptor involved in endothelial cell specific signaling. EPCR binds APC and is a coreceptor for cleavage of PAR1 resulting in endothelial cell protective signaling within the protective protein C pathway.

The term “protease-activated receptor (PAR),” refers to a seven transmembrane domain G-protein-coupled receptor. PAR1 is activated by proteolysis by either APC or thrombin. APC is shown to use endothelial protein C receptor (EPCR) as a coreceptor to cleave PAR1 and activate PAR1 on endothelial cells. Gene profiling demonstrated that APC/EPCR/PAR1 signaling could account for all APC-induced protective genes, including, for example, monocyte chemoattractant protein-1 (MCP-1).

Thrombin binds to N-terminal exodomain of PAR1 and cleaves it after Arg41 to generate a new receptor N-terminus. PAR1, PAR3, and PAR4 can be activated by thrombin, and thrombin is almost certainly a physiological activator of these receptors in vivo. Several other proteases can cleave these receptors productively and can also contribute to their function in vivo. APC can cleave PAR1 and activate the receptor. PAR2 can be activated by multiple trypsin-like serine proteases including: trypsin itself, mast cell tryptase, neutrophil proteinase 3, tissue factor/factor VIIa/factor Xa, and membrane-tethered serine protease-1. However PAR2 is not cleaved by thrombin.

The term “protease activated receptor (PAR)-1 pathway”, “protective protein C (PC) pathway” or “PAR1 dependent signaling” refers to activation of the PAR1 receptor by APC and EPCR within the protective protein C (PC) pathway to counterbalance or block thrombin induced inflammatory response and provide protection from sepsis.

The term “APC/EPCR/PAR1 induced protective gene” or “APC/EPCR/PAR1 induced protective protein” refers to expression of genes, proteins or polypeptides that are induced, activated, inactivated or repressed by the APC/EPCR/PAR1 signaling pathway resulting in a reduced inflammatory response and prevention of sepsis in a mammalian subject.

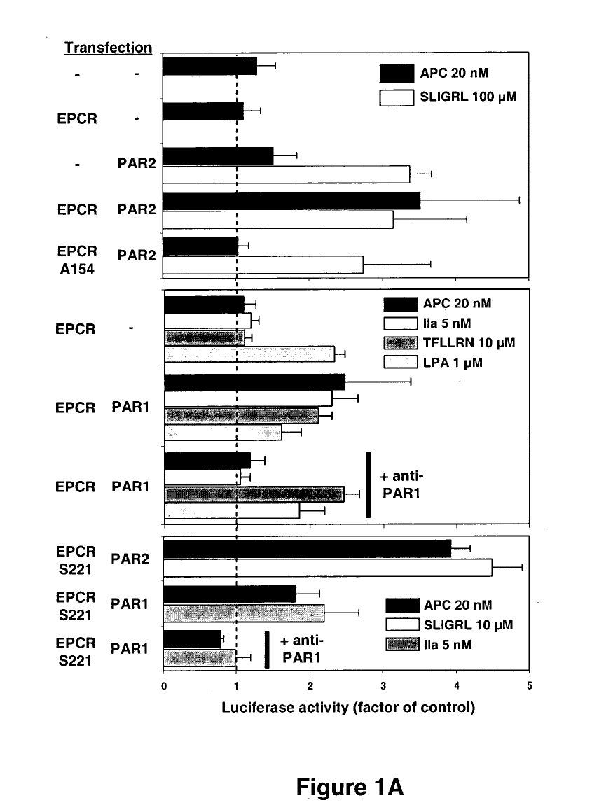

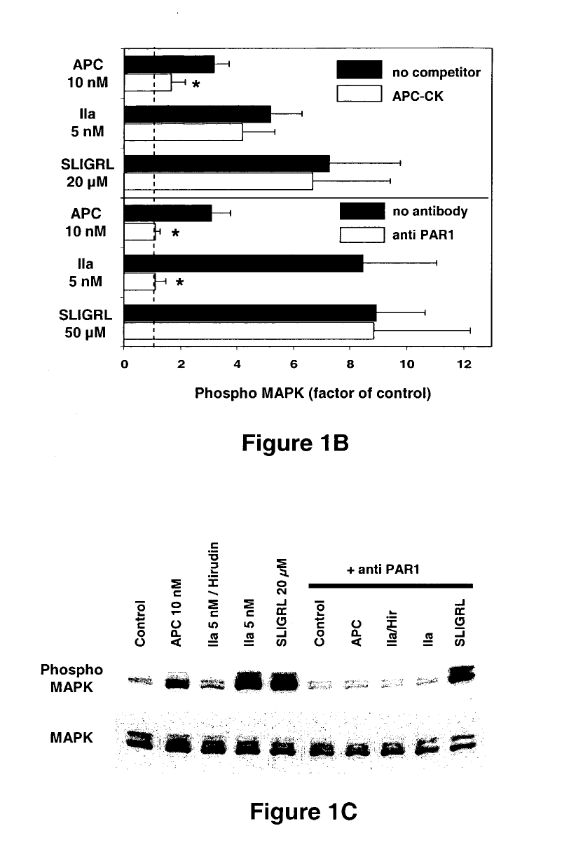

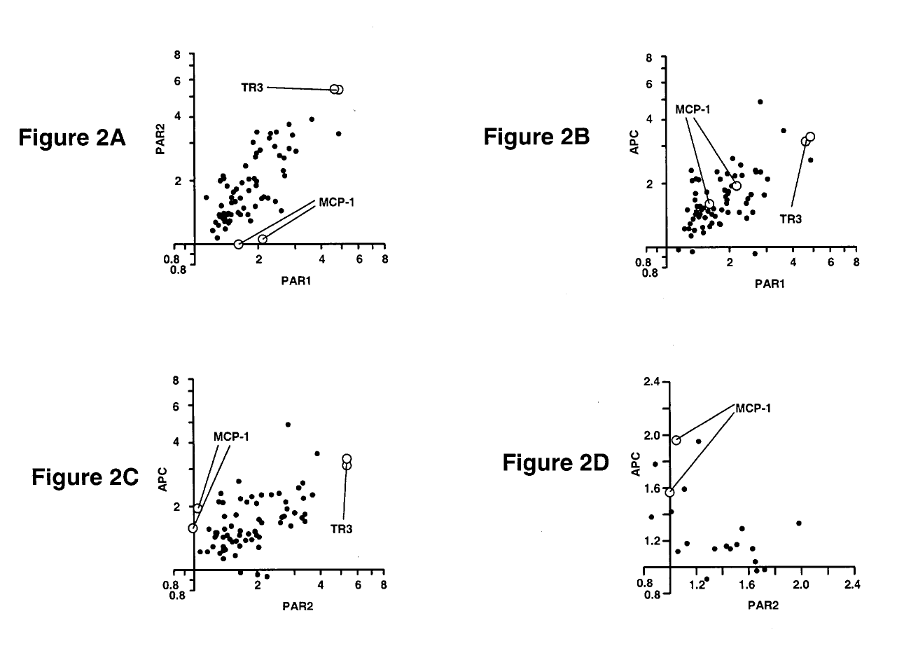

The term “cell-based assay system” refers to an in vitro cell assay system utilizing a cell line, for example, fibroblast cells or endothelial cells, expressing EPCR, PAR1, and/or PAR2 to assay for endothelial cell protective signaling within the protective protein C pathway. Examples of cell based assay systems include, but are not limited to, (1) induction of Egr-1 promoter-luciferase reporter activity in PAR1-deficient fibroblasts transfected with human EPCR, EPCR variants, human PAR2 or human PAR1. Fold induction of luciferase activity is measured upon stimulation with APC, PAR1 agonist peptide, PAR2 agonist peptide, or thrombin. See FIGS. 1A and 1B; (2) APC signaling in human umbilical vein endothelia cells (HUVECs) measured as Erk 1/2 phosphorylation. Stimulation with PAR1 agonist, PAR2 agonist, or thrombin. See FIGS. 1C and 1D; (3) APC- and PAR1-specific agonist induced a panel of genes in human endothelial cells by a gene array assay. See FIG. 2.

The cell-based assay system can be performed in any cell type that expresses PAR1 and EPCR, either endogenously or by cells transfected with PAR1 and EPCR DNA expression vectors. PAR1 is expressed on a wide variety of cells and APC-EPCR-PAR1 signaling can occur virtually in all cells that express EPCR. Although EPCR is highly specific for endothelial cells, EPCR expression has been demonstrated on placental trophoblast cells (Crawley, J. T., et al., Thromb Haemost 88: 259-66, 2002), monocytes and monocytic cell lines (Galligan, L., et al., Br J Haematol 115: 408-14, 2001), and various human tumor cell types (Tsuneyoshi, N., Thromb Haemost 85: 356-61, 2001; Scheffer, G. L., et al., Eur J Cancer 38: 1535-42, 2002). In addition soluble EPCR can be recruited to activated neutrophil leukocytes and the APC-EPCR-PAR1 signaling pathway can also be operative on these cells (Kurosawa, S., et al., J Immunol 165: 4697-703, 2000).

The term “measuring a reporter gene activity” refers to measurement of a gene product, nucleic acid or protein wherein the gene is often of prokaryotic origin that produces a product easily detected in eukaryotic cells and that is used as a marker to determine the activity of another gene with which its DNA has been closely linked or combined. The reporter gene activity includes, but is not limited to, luciferase, chloramphenicol acetyl transferase, or green fluorescent protein. The reporter gene activity is generally a measure of gene expression at the level of gene transcription as a result of signaling through the APC/EPCR/PAR1 pathway.

The term “test compound” refers to a nucleic acid, DNA, RNA, protein, polypeptide, or small chemical entity that is determined to effect an increase or decrease in a gene expression as a result of signaling through the APC/EPCR/PAR1 pathway. The test compound can be an antisense RNA, ribozyme, polypeptide, or small molecular chemical entity. The term “test compound” can be any small chemical compound, or a biological entity, such as a protein, sugar, nucleic acid or lipid. Typically, test compounds will be small chemical molecules and polypeptides. A “test compound specific for signaling by APC or PAR1” is determined to be a modulator of a APC/EPCR/PAR1 induced protective protein.

The term “modulate gene expression” or “modulate signaling” refers to an increase or decrease in a gene expression product, RNA or protein, as a result of signaling through the APC/EPCR/PAR1 pathway.

The term “gene activation state”, “up-regulation” or “down-regulation” refers to modulation of gene expression in a cell or tissue treated with a test compound or effector molecule resulting in an increase or decrease in a gene expression product, RNA or protein, as a result of signaling through the APC/EPCR/PAR1 pathway. “Gene activation state” can be measured on a gene expression panel microarray by measuring fold increase or decrease of a gene in the microarray derived from a tissue treated with a test compound compared to a tissue untreated with a test compound. “Gene activation state” can also be measured by a reporter gene activity, for example, Egr-1 promoter/luciferase activity in a PAR1 deficient fibroblast cell transfected with human EPCR; or in human umbilical vein endothelial cells (HUVECs) measured as Erk1/2 phosphorylation. “Up regulation” of a gene activation state or “activation of a level of gene expression” refers to an increase in gene expression within a cell or tissue treated with a test compound or effector molecule that results in an approximately 1.3-fold or greater increase in gene activation as measured by a gene expression product, RNA or protein, as a result of signaling through the APC/EPCR/PAR1 pathway. “Down regulation” of a gene activation state or “repression of a level of gene expression” refers to an decrease in gene expression in a cell or tissue treated with a test compound or effector molecule that results in a gene activation state that is below approximately 0.7-fold as measured by a gene expression product, RNA or protein, as a result of signaling through the APC/EPCR/PAR1 pathway. The gene activation state can be considered unchanged if the difference in gene expression is approximately 1.0-fold, or typically, approximately 0.7-fold to 1.3-fold as measured by a gene expression product, RNA or protein, as a result of signaling through the APC/EPCR/PAR1 pathway.

The term “contacting” refers to mixing a test compound in a soluble form into a cell-based assay system such that an effect upon PAR1 receptor mediated signaling can be measured.

The term “signaling responsiveness” or “effective to activate signaling” or “stimulating a cell-based assay system” refers to the ability of APC to use EPCR as a co-receptor for proteolytic cleavage of the PAR1 receptor thus activating PAR1 and inducing a protective response in endothelial cells against inflammation and sepsis.

The term “detecting an effect” refers to an effect measured in a cell-based assay system. For example, the effect detected can be measured in PAR1-deficient fibroblasts transfected with human EPCR measuring fold-induction of Egr-1 promoter activity (luciferase reporter) upon stimulation with APC or an agonist. The effect detected can be APC signaling in human umbilical vein endothelia cells (HUVECs) measured as Erk 1/2 phosphorylation. Furthermore, the effect detected can be APC- and PAR1-specific agonist induced a panel of genes in human endothelial cells by a gene array assay.

The term “assay being indicative of modulation” refers to results of a cell-based assay system indicating that cell activation by APC uses EPCR as a coreceptor for cleavage of PAR1 receptor and thus activating PAR1. PAR1 activation induces a protective response in endothelial cells against inflammation and sepsis.

The term “inflammation” or “inflammatory response” refers to an innate immune response that occurs when tissues are injured by bacteria, trauma, toxins, heat, or any other cause. The damaged tissue releases compounds including histamine, bradykinin, and serotonin. Inflammation refers to both acute responses (i.e., responses in which the inflammatory processes are active) and chronic responses (i.e., responses marked by slow progression and formation of new connective tissue). Acute and chronic inflammation can be distinguished by the cell types involved. Acute inflammation often involves polymorphonuclear neutrophils; whereas chronic inflammation is normally characterized by a lymphohistiocytic and/or granulomatous response. Inflammation includes reactions of both the specific and non-specific defense systems. A specific defense system reaction is a specific immune system reaction response to an antigen (possibly including an autoantigen). A non-specific defense system reaction is an inflammatory response mediated by leukocytes incapable of immunological memory. Such cells include granulocytes, macrophages, neutrophils and eosinophils. Examples of specific types of inflammation are diffuse inflammation, focal inflammation, croupous inflammation, interstitial inflammation, obliterative inflammation, parenchymatous inflammation, reactive inflammation, specific inflammation, toxic inflammation and traumatic inflammation.

The term “sepsis” or systemic inflammatory response syndrome (SIRS) refers to a disease that occurs in 2 of every 100 hospital admissions. Sepsis is caused by bacterial infection that can originate anywhere in the body. Common sites of sepsis include the kidneys, liver, gall bladder, bowel, skin, or lungs. Meningitis can also be accompanied by sepsis. In children, sepsis can accompany infection of the bone (osteomyelitis).

The term “ischemic injury” refers to, for example, ischemic cardiomyopathy that results from blockage of coronary arteries leading to destruction of heart muscle tissue. Ischemic cardiomyopathy is a common cause of congestive heart failure. Patients with ischemic cardiomyopathy can at one time have had acute heart attact, angina, or unstable angina.

The term “stroke” refers to an interruption of the blood supply to any part of the brain resulting in ischemic injury or tissue death and loss of brain function. Stroke can occur as a result of a blood clot or atherosclerosis. The risk of stroke is increased by smoking, hypertension, diabetes, hyperlipidemia, and heart disease.

The term “apoptosis” refers to programmed cell death. Apoptosis refers to a genetically determined process of cell self-destruction that is marked by the fragmentation of nuclear DNA. Apoptosis is activated either by the presence of a stimulus or by the removal of a stimulus or suppressing agent. It is a normal physiological process eliminating DNA-damaged, superfluous, or unwanted cells (as immune cells targeted against the self in the development of self-tolerance or larval cells in amphibians undergoing metamorphosis), and when halted (as by genetic mutation) can result in uncontrolled cell growth and tumor formation

Gene Expression Profiles

The present methods screen for compositions which modulate endothelial cells. The expression levels of genes are determined for different cellular states of endothelial cells to provide expression profiles. An endothelial expression profile of a particular endothelial cell state can be a “fingerprint” of the state; while two states can have any particular gene similarly expressed, the evaluation of a number of genes simultaneously allows the generation of a gene expression profile that is unique to the state of the cell. By comparing expression profiles of endothelial cells in an anti-inflammatory state compared to a state of coagulant and inflammatory exacerbation in sepsis, information regarding which genes are important (including both up- and down-regulation of genes) in each of these states is obtained. This information can then be used in a number of ways. For example, the evaluation of a particular treatment regime can be evaluated: does an anti-inflammatory drug act like an anti-inflammatory drug in this particular patient according to a gene expression profile. Similarly, diagnosis can be done or confirmed: does this patient have the gene expression profile of anti-inflammatory endothelial cells. Furthermore, these gene expression profiles can be used in drug candidate screening to find drugs that mimic a particular expression profile; for example, screening can be done for drugs that induce an anti-inflammatory expression profile similar to APC using EPCR as a coreceptor for cleavage of PAR1 on endothelial cells. Accordingly, genes are identified and described which are differentially expressed within and among endothelial cells in different states, from which the expression profiles are generated as further described herein. For example, determinations of differentially expressed nucleic acids are provided herein for endothelial cells which are in an anti-inflammatory state or in a state of coagulant and inflammatory exacerbation in sepsis.

“Differential expression,” or grammatical equivalents as used herein, refers to both qualitative as well as quantitative differences in the temporal and/or cellular expression patterns of the genes within and among endothelial cells. Thus, a differentially expressed gene can qualitatively have its expression altered, including an activation or inactivation of a state of an endothelial cell, for example, tissue factor-initiated and thrombin-initiated coagulation and inflammatory exacerbation in sepsis can trigger a lethal response mediated by endothelial cells, or the protective protein (PC) pathway can be mediated by activated protein C (APC), endothelial cell PC receptor (EPCR) and protease activated receptor (PAR)-1 in endothelial cells. Genes can be turned on or turned off in a particular state, relative to another state. Any comparison of two or more states can be made. Such a qualitatively regulated gene will exhibit an expression pattern within a state or cell type which can be detectable by standard techniques in one such state or cell type, but can be not detectable in both. Alternatively, the determination can be quantitative in that expression is increased or decreased; that is, the expression of the gene is either upregulated, resulting in an increased amount of transcript, or downregulated, resulting in a decreased amount of transcript. The degree to which expression differs need only be large enough to quantify using standard characterization techniques, for example, by using Affymetrix GeneChip™ expression arrays (Lockhart, Nature Biotechnology, 14:1675-1680, 1996; this reference and all references cited therein are incorporated by reference). Other methods include, but are not limited to, quantitative reverse transcriptase PCR, Northern analysis and RNase protection. The change or modulation in expression (i.e., upregulation or downregulation) is at least about 5%, typically at least about 10%, typically, at least about 20%, at least about 30%, or typically by at least about 50%, or at least about 75%, and typically at least about 90%.

Any one, two, three, four, five, or ten or more genes can be evaluated in the evaluation of the basis for an inflammatory response and sepsis and blocking such a response. Table 3 shows the following unchanged or down-regulated by APC-PAR1 signaling and up-regulated by thrombin signaling. For example, in developing a therapeutic treatment for sepsis or inflammation, the following gene targets can be down-regulated to prevent sepsis and/or an inflammatory response. These genes include, but are not limited to, SH-PTP3, W28170, fructose-6-phosphate,2-kinase/fructose-2,6-bisphosphatase, W28616, BID, NF-KB2, thrombospondin-1,6-phosphofructo-2-kinase/fructose-2,6-bisphosphatase, neurofibromatosis 2 tumor suppressor, cationic amino acid transporter 2, W28616, NF-kappa-B p65delta3, TTF-I interacting peptide 12, ATRC2, alpha adducin, sortilin, p53 cellular tumor antigen, W28257, hERRa1, G protein-coupled receptor, EGFRBP-GRB2, Pax8, SH-PTP3, 5T4, bc1-xL, disintegrin-metalloprotease, C1q-related factor, cyclin D1, A1743606, myosin-IXb, GNS, ISLR, Stat2, stem cell factor, c-ets-1, usurpin-beta, chromosome 5q21-22, clone-A3-A, thrombospondin, ELL2, dual-specificity protein phosphatase, vitronectin receptor alpha subunit, PCTAIRE-2, follistatin, ets-2, ELL2, utrophin, C8FW phosphoprotein, PCTAIRE-2, fra-2, MINOR, GADD34, IL8, SSR alpha, neuron derived orphan receptor, CGGBP, nma, jun-B, epsilon tyrosine phosphatase, GRO-beta, CtIP, PRDII-BF1, vascular endothelial growth factor, stanniocalcin-related protein, CL 100, BMP-2A, NF-ATc, mrg1, jun-B, VEGF, STC, and ATF3. The accession numbers for these genes can be found in Table 3. This panel of genes is downregulated or repressed in cells treated with APC as a result of APC/EPCR/PAR1 signaling pathway; but up-regulated in cells treated with thrombin.

Any one, two, three, four, five, or ten or more genes can be evaluated in the evaluation of the basis for an inflammatory response and sepsis and blocking such a response. Table 1 shows the following genes that are unchanged by PAR2 agonist signaling and upregulated by PAR1 agonist signaling or APC signaling in endothelial cells. For example, in developing a therapeutic treatment for sepsis or inflammation, the following gene targets can be up-regulated to prevent sepsis and/or an inflammatory response. Additional genes to be evaluated include, but are not limited to MCP-1, qe82d12.x1, RACH1, PAC747L4, T-plastin, endothelin receptor type B-like, VEGF, and wx69d10.x1. The accession numbers for these genes can be found in Table 1. This panel of genes is up-regulated or induced by PAR1 receptor agonist or APC, but not induced by PAR2 receptor agonist. Generally, oligonucleotide sequences used in the evaluation of these genes are derived from their 3′ untranslated regions.

Differentially expressed genes can represent “expression profile genes”, which includes “target genes”. “Expression profile gene,” as used herein, refers to a differentially expressed gene whose expression pattern can be used in methods for identifying compounds useful in the modulation of endothelial cell states or activity, or the treatment of disorders, or alternatively, the gene can be used as part of a prognostic or diagnostic evaluation of immune disorders. For example, the effect of the compound on the expression profile gene normally displayed in connection with a particular state, such as anti-inflammatory response, for example, can be used to evaluate the efficacy of the compound to modulate that state, or to induce or maintain that state. Such assays are further described below. Alternatively, the gene can be used as a diagnostic or in the treatment of inflammatory disorders or sepsis as also further described below. In some instances, only a fragment of an expression profile gene is used, as further described below.

“Expression profile,” as used herein, refers to the pattern of gene expression generated from two up to all of the expression profile genes which exist for a given state. As outlined above, an expression profile is in a sense a “fingerprint” or “blueprint” of a particular cellular state; while two or more states have genes that are similarly expressed, the total expression profile of the state will be unique to that state. The gene expression profile obtained for a given endothelial cell state can be useful for a variety of applications, including diagnosis of a particular disease or condition and evaluation of various treatment regimes. In addition, comparisons between the expression profiles of different endothelial cell states can be similarly informative. An expression profile can include genes which do not appreciably change between two states, so long as at least two genes which are differentially expressed are represented. The gene expression profile can also include at least one target gene, as defined below. Alternatively, the profile can include all of the genes which represent one or more states. Specific expression profiles are described below.

Gene expression profiles can be defined in several ways. For example, a gene expression profile can be the relative transcript level of any number of particular set of genes. Alternatively, a gene expression profile can be defined by comparing the level of expression of a variety of genes in one state to the level of expression of the same genes in another state. For example, genes can be either upregulated, downregulated, or remain substantially at the same level in both states.

A gene expression profile for cells responding to anti-inflammatory therapy can include a combination of at least two of SH-PTP3, W28170, fructose-6-phosphate,2-kinase/fructose-2,6-bisphosphatase, W28616, BID, NF-KB2, thrombospondin-1,6-phosphofructo-2-kinase/fructose-2,6-bisphosphatase, and neurofibromatosis 2 tumor suppressor. This expression profile is for genes down-regulated in the presence of APC and up-regulated in the presence of thrombin. Another gene expression profile for cells responding to anti-inflammatory therapy can include MCP-1. This expression profile is for genes up-regulated by APC or PAR1 agonist and unchanged by PAR2 agonist.

Target and Pathway Genes

In addition to expression profile genes, target genes are also provided. “Target gene,” as used herein, refers to a differentially expressed expression profile gene whose expression is unique for a particular state, such that the presence or absence of the transcript of a target gene(s) can indicate the state the cell is in. A target gene can be completely unique to a particular state; the presence or absence of the gene is only seen in a particular cell state, or alternatively, cells in all other states express the gene but it is not seen in the first state. Thus for example, expression of SH-PTP3 can be an indicator of sepsis or of an inflammatory state. Expression of SH-PTP3 is downregulated in the presence of APC and upregulated in the presence of thrombin. Alternatively, target genes can be identified as relevant to a comparison of two states, that is, the state is compared to another particular state or standard to determine the uniqueness of the target gene. Target genes can be used in the diagnostic, prognostic, and compound identification methods described herein.

It should be understood that a target gene for a first state can be an expression profile gene for a second state. The presence or absence of a particular target gene in one state can be diagnostic of the state; the same gene in a different state can be an expression profile gene.

Further, pathway genes are provided herein. “Pathway genes” are defined by the ability of their gene products to interact with expression profile genes. Pathway genes can also exhibit target gene and/or expression profile gene characteristics and can be included as modulators of expression profile genes as further described below.

The products of such expression profile, target, and pathway genes, as well as antibodies to such gene products are also included. Furthermore, the engineering and use of cell- and animal-based models of endothelial cell states to which such profiles, genes and gene products can contribute, are also described.

Use of Gene Expression Monitoring for Genetic Network Mapping and Gene Function Identification

Methods, compositions and apparatus for interrogating the genetic network and for studying normal and abnormal functions for specific genes are provided. The methods involve quantifying the level of expression of a large number of genes. In some embodiments, a high density oligonucleotide array is used to hybridize with a target nucleic acid sample to detect the expression level of a large number of genes, typically more than 10, more than 100, and typically more than 1000 genes.

A variety of nucleic acid samples are prepared according to these methods to represent many states of the genetic network. By comparing the expression levels of those samples, regulatory relationships among genes can be determined with a certain statistical confidence. A dynamic map can be constructed based upon expression data.

Such a genetic network map is extremely useful for drug discovery. For example, if a gene of interest is found to be associated with a particular disease, a list of potential up-stream regulatory genes can be found using such a genetic network map. Research efforts can then be concentrated on the potential up-stream genes as drug targets. Similarly, if a gene mutation causes a disease, it can affect genes that are both related and unrelated to the pathogenesis of the disease. The relationships can be explored to find the pathogenic genes. In such embodiments, the association between a disease state and the expression of a large number of genes is determined, and the genes whose expression is altered in the diseased tissue are identified. The up-stream genes that regulate the altered genes are indicated as functionally altered or potentially mutated.

In general, genetic regulatory relationships can be explored to detect potential mutations once a target gene's down-stream regulated genes are identified. In one embodiment, the expression of several down-stream positively regulated genes is monitored using a high density oligonucleotide array. Diminished expression of those positively regulated genes indicates a possible malfunction of the target gene. Such malfunction can indicate the presence of a potential mutation in the target gene. Other mutation detection methods, such as the tiling methods, can then be used to confirm and to detect the nature of the mutation. Many sets of such down-stream positively regulated genes, each set of genes being regulated by a target gene, can be monitored simultaneously. This simultaneous detection of mutations in many genes is an major improvement over prior art methods. It will be apparent to those skilled in the art that negatively regulated down-stream genes can also be used in a similar manner.

Similarly, in some embodiments, the regulatory function of a particular gene can be identified by monitoring a large number of genes. In a further embodiment, the expression of a gene of interest is suppressed by applying antisense oligonucleotides. The expression of a large number of genes are monitored to provide an expression pattern. The expression of the gene of interest is then restored and the expression of a large number of genes are similarly monitored to provide another expression pattern. By comparing the expression patterns, the regulatory function of the gene of interest can be deduced.

Detecting the Regulation of Gene Expression

Activity of a gene is reflected by the activity of its product(s): the proteins or other molecules encoded by the gene. Those product molecules perform biological functions. Directly measuring the activity of a gene product is, however, often difficult for certain genes. Instead, the anti-inflammatory activities or the amount of the final product(s) or its peptide processing intermediates are determined as a measurement of the gene activity. More frequently, the amount or activity of intermediates, such as transcripts, RNA processing intermediates, or mature mRNAs are detected as a measurement of gene activity.

In many cases, the form and function of the final product(s) of a gene is unknown. In those cases, the activity of a gene is measured conveniently by the amount or activity of transcript(s), RNA processing intermediate(s), mature mRNA(s) or its protein product(s) or functional activity of its protein product(s).

Any methods that measure the activity of a gene are useful for at least some embodiments. For example, traditional Northern blotting and hybridization, nuclease protection, RT-PCR and differential display have been used for detecting gene activity. Those methods are useful for some embodiments. However, these methods are most useful in conjunction with methods for detecting the expression of a large number of genes.

High density arrays are particularly useful for monitoring the expression control at the transcriptional, RNA processing and degradation level. The fabrication and application of high density arrays in gene expression monitoring have been disclosed previously in, for example, WO 97/10365, WO 92/10588, U.S. Pat. No. 6,309,822; U.S. Pat. No. 6,040,138; U.S. Ser. No. 08/168,904 filed Dec. 15, 1993; Ser. No. 07/624,114 filed on Dec: 6, 1990, Ser. No. 07/362,901 filed Jun. 7, 1990, each incorporated herein by reference. In some embodiment using high density arrays, high density oligonucleotide arrays are synthesized using methods such as the Very Large Scale Immobilized Polymer Synthesis (VLSIPS) disclosed in U.S. Pat. No. 5,445,934, incorporated herein by reference. Each oligonucleotide occupies a known location on a substrate. A nucleic acid target sample is hybridized with a high density array of oligonucleotides and then the amount of target nucleic acids hybridized to each probe in the array is quantified. One quantifying method is to use confocal microscope and fluorescent labels. The GeneChip™ system (Affymetrix, Santa Clara, Calif.) is particularly suitable for quantifying the hybridization; however, it will be apparent to those of skill in the art that any similar systems or other effectively equivalent detection methods can also be used.

High density arrays are suitable for quantifying a small variations in expression levels of a gene in the presence of a large population of heterogeneous nucleic acids. Such high density arrays can be fabricated either by de novo synthesis on a substrate or by spotting or transporting nucleic acid sequences onto specific locations of substrate. Nucleic acids are purified and/or isolated from biological materials, such as a bacterial plasmid containing a cloned segment of sequence of interest. Suitable nucleic acids are also produced by amplification of templates. As a nonlimiting illustration, polymerase chain reaction, and/or in vitro transcription, are suitable nucleic acid amplification methods.

Synthesized oligonucleotide arrays are particularly useful for the methods herein. Oligonucleotide arrays have numerous advantages, as opposed to other methods, such as efficiency of production, reduced intra- and inter array variability, increased information content and high signal-to-noise ratio.

One of skill in the art would appreciate that in order to interrogate the genetic network, it is desirable to measure the control of transcription. Because all the cell nuclei of an organism generally carry the same genes, the difference in the protein products in different cell types is generally the result of selective gene expression. It is well known in the art that the first level of regulation is at the level of transcription, i.e., by varying the frequency with which a gene is transcribed into nascent pre-mRNA by a RNA polymerase. The regulation of transcription is one of the most important steps in the control of gene expression because transcription constitutes the input of the mRNA pool. It is generally known in the art that transcriptional regulation can be achieved through various means. As non-limiting examples, transcription can be controlled by a) cis-acting transcriptional control sequences and transcriptional factors; b) different gene products from a single transcription unit, c) epigenetic mechanisms; and d) long range control of genetic expression by chromatin structure. Methods for detecting the transcriptional regulation of individual genes at all of these levels of control are provided.

Massive Parallel Gene Expression Monitoring

One method for massive parallel gene expression monitoring is based upon high density nucleic acid arrays. Nucleic acid array methods for monitoring gene expression are disclosed and discussed in detail in PCT Application WO 92/10588, incorporated herein by reference.

Generally those methods of monitoring gene expression involve (a) providing a pool of target nucleic acids comprising RNA transcript(s) of one or more target gene(s), or nucleic acids derived from the RNA transcript(s); (b) hybridizing the nucleic acid sample to a high density array of probes and (c) detecting the hybridized nucleic acids and calculating a relative and/or absolute expression (transcription, RNA processing or degradation) level.

One of skill in the art will appreciate that it is desirable to have nucleic acid samples containing target nucleic acid sequences that reflect the transcripts of interest. Therefore, suitable nucleic acid samples can contain transcripts of interest or can contain nucleic acids derived from the transcripts of interest. As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable samples include, but are not limited to, transcripts of the gene or genes, cDNA reverse transcribed from the transcript, cRNA transcribed from the cDNA, DNA amplified from the genes, RNA transcribed from amplified DNA, and the like. Transcripts, as used herein, can include, but are not limited to pre-mRNA nascent transcript(s), transcript processing intermediates, mature mRNA(s) and degradation products. It is not necessary to monitor all types of transcripts. For example, one can choose to measure the mature mRNA levels only. See, for example, U.S. Pat. No. 6,340,565, U.S. patent application 20010031462, each incorporated herein by reference.

Other suitable amplification methods include, but are not limited to polymerase chain reaction (PCR) (Innis, et al., PCR Protocols. A Guide to Methods and Application. Academic Press, Inc. San Diego, 1990), ligase chain reaction (LCR) (Wu and Wallace, Genomics, 4: 560, 1989, Landegren, et al., Science, 241: 1077, 1988, and Barringer, et al., Gene, 89: 117, 1990) transcription amplification (Kwoh, et al., Proc. Natl. Acad. Sci. USA, 86: 1173, 1989), and self-sustained sequence replication (Guatelli, et al., Proc. Nat. Acad. Sci. USA, 87: 1874, 1990). Each citation incorporated herein by reference.

Cell lysates or tissue homogenates often contain a number of inhibitors of polymerase activity. Therefore, RT-PCR typically incorporates preliminary steps to isolate total RNA or mRNA for subsequent use as an amplification template. A one-tube mRNA capture method can be used to prepare poly(A)⁺ RNA samples suitable for immediate RT-PCR in the same tube (Boehringer Mannheim). The captured mRNA can be directly subjected to RT-PCR by adding a reverse transcription mix and, subsequently, a PCR mix.

In one embodiment, the sample mRNA is reverse transcribed with a reverse transcriptase and a primer consisting of oligo(dT) and a sequence encoding the phage T7 promoter to provide single stranded DNA template. The second DNA strand is polymerized using a DNA polymerase. After synthesis of double-stranded cDNA, T7 RNA polymerase is added and RNA is transcribed from the cDNA template. Successive rounds of transcription from each single cDNA template results in amplified RNA. Methods of in vitro polymerization are well known to those of skill in the art (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987), incorporated herein by reference.) and this particular method is described in detail by Van Gelder, et al., Proc. Natl. Acad. Sci. USA, 87: 1663-1667, 1990, incorporated herein by reference, who demonstrate that in vitro amplification according to this method preserves the relative frequencies of the various RNA transcripts. Moreover, Eberwine et al. Proc. Natl. Acad. Sci. USA, 89: 3010-3014, incorporated herein by reference, provide a protocol that uses two rounds of amplification via in vitro transcription to achieve greater than 10⁶ fold amplification of the original starting material, thereby permitting expression monitoring even where biological samples are limited.

It will be appreciated by one of skill in the art that the direct transcription method described above provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes can be of either sense as the target nucleic acids include both sense and antisense strands.

The protocols cited above include methods of generating pools of either sense or antisense nucleic acids. Indeed, one approach can be used to generate either sense or antisense nucleic acids as desired. For example, the cDNA can be directionally cloned into a vector (e.g., Stratagene's p Bluscript II KS (+) phagemid) such that it is flanked by the T3 and T7 promoters. In vitro transcription with the T3 polymerase will produce RNA of one sense (the sense depending on the orientation of the insert), while in vitro transcription with the T7 polymerase will produce RNA having the opposite sense. Other suitable cloning systems include phage lambda vectors designed for Cre-loxP plasmid subcloning (see e.g., Palazzolo et al., Gene, 88: 25-36, 1990, incorporated herein by reference).

A variety of labels can be incorporated into target nucleic acids in the course of amplification or after amplification. Suitable labels include fluorescein or biotin, the latter being detected by staining with phycoerythrin-streptavidin after hybridization. In some methods, hybridization of target nucleic acids is compared with control nucleic acids. Optionally, such hybridizations can be performed simultaneously using different labels are used for target and control samples. Control and target samples can be diluted, if desired, prior to hybridization to equalize fluorescence intensities.

Supports

Supports can be made of a variety of materials, such as glass, silica, plastic, nylon or nitrocellulose. Supports can be rigid and have a planar surface. Supports typically have from 1-10,000,000 discrete spatially addressable regions, or cells. Supports having 10-1,000,000 or 100-100,000 or 1000-100,000 cells are common. The density of cells is typically at least 1000, 10,000, 100,000 or 1,000,000 cells within a square centimeter. Typically a single probe per cell. In some supports, all cells are occupied by pooled mixtures of probes. In other supports, some cells are occupied by pooled mixtures of probes, and other cells are occupied, at least to the degree of purity obtainable by synthesis methods, by a single type of polynucleotide. The strategies for probe design described in the present application can be combined in the same array with other strategies, such as those described by WO 95/11995, EP 717,113 and WO 97/29212, each incorporated herein by reference.

The location and sequence of each different polynucleotide probe in the array is generally known. Moreover, the large number of different probes can occupy a relatively small area providing a high density array having a probe density of generally greater than about 60, more generally greater than about 100, and most generally greater than about 600, often greater than about 1000, more often greater than about 5,000, most often greater than about 10,000, typically greater than about 40,000 greater than about 100,000, and typically greater than about 400,000 different polynucleotide probes per cm². The small surface area of the array (often less than about 10 cm², less than about 5 cm² less than about 2 cm², and typically less than about 1.6 cm²) permits the use of small sample volumes and extremely uniform hybridization conditions.

Synthesis of Probe Arrays

Arrays of probes can be synthesized in a step-by-step manner on a support or can be attached in presynthesized form. One method of synthesis is VLSIPS™ (see Fodor et al., 1993, Nature 364, 555-556; McGall et al., U.S. S. N. 08/445,332; U.S. Pat. No. 5,143,854; EP 476,014, each incorporated herein by reference), which entails the use of light to direct the synthesis of polynucleotide probes in high-density, miniaturized arrays. Algorithms for design of masks to reduce the number of synthesis cycles are described by Hubbel et al., U.S. Pat. No. 5,571,639 and U.S. Pat. No. 5,593,839, each incorporated herein by reference. Arrays can also be synthesized in a combinatorial fashion by delivering monomers to cells of a support by mechanically constrained flowpaths. Arrays can also be synthesized by spotting monomers reagents on to a support using an ink jet printer. See EP 624,059, EP 728,520, each incorporated herein by reference.

After hybridization of control and target samples to an array containing one or more probe sets as described above and optional washing to remove unbound and nonspecifically bound probe, the hybridization intensity for the respective samples is determined for each probe in the array. For fluorescent labels, hybridization intensity can be determined by, for example, a scanning confocal microscope in photon counting mode. Appropriate scanning devices are described by e.g., U.S. Pat. No. 5,578,832; U.S. Pat. No. 5,631,734, each incorporated herein by reference, and are available from Affymetrix, Inc., under the GeneChip™ label. Some types of label provide a signal that can be amplified by enzymatic methods. See Broude, et al. Proc. Natl. Acad. Sci. U.S.A. 91: 3072-3076, 1994, incorporated herein by reference.

Design of Arrays

(A) Customized and Generic Arrays

The design of arrays for expression monitoring is generally described, for example, WO 97/27317 and WO 97/10365, each incorporated herein by reference. There are two principal categories of arrays. One type of array detects the presence and/or levels of particular mRNA sequences that are known in advance. In these arrays, polynucleotide probes can be selected to hybridize to particular preselected subsequences of mRNA gene sequence. Such expression monitoring arrays can include a plurality of probes for each mRNA to be detected. For analysis of mRNA nucleic acids, the probes are designed to be complementary to the region of the mRNA that is incorporated into the nucleic acids (i.e., the 3′ end). The array can also include one or more control probes.

Generic arrays can include all possible nucleotides of a given length; that is, polynucleotides having sequences corresponding to every permutation of a sequence. Thus since the polynucleotide probes of the methods herein include up to 4 bases (A, G, C, T) or (A, G, C, U) or derivatives of these bases, an array having all possible nucleotides of length X contains substantially 4^{X} different nucleic acids (e.g., 16 different nucleic acids for a 2 mer, 64 different nucleic acids for a 3 mer, 65536 different nucleic acids for an 8 mer). Some small number of sequences can be absent from a pool of all possible nucleotides of a particular length due to synthesis problems, and inadvertent cleavage). An array comprising all possible nucleotides of length X refers to an array having substantially all possible nucleotides of length X. All possible nucleotides of length X includes more than 90%, typically more than 95%, more than 98%, more more than 99%, and typically more than 99.9% of the possible number of different nucleotides. Generic arrays are particularly useful for comparative hybridization analysis between two mRNA populations or nucleic acids derived therefrom.

(B) Control Probes

Either customized or generic probe arrays can contain control probes in addition to the probes described above.

Normalization controls are typically perfectly complementary to one or more labeled reference polynucleotides that are added to the nucleic acid sample. The signals obtained from the normalization controls after hybridization provide a control for variations in hybridization conditions, label intensity, reading and analyzing efficiency and other factors that can cause the signal of a perfect hybridization to vary between arrays. Signals (e.g., fluorescence intensity) read from all other probes in the array can be divided by the signal (e.g., fluorescence intensity) from the control probes thereby normalizing the measurements.

Virtually any probe can serve as a normalization control. However, hybridization efficiency can vary with base composition and probe length. Normalization probes can be selected to reflect the average length of the other probes present in the array, however, they can also be selected to cover a range of lengths. The normalization control(s) can also be selected to reflect the (average) base composition of the other probes in the array. However one or a fewer normalization probes can be used and they can be selected such that they hybridize well (i.e., no secondary structure) and do not match any target-specific probes.

Normalization probes can be localized at any position in the array or at multiple positions throughout the array to control for spatial variation in hybridization efficiently. The normalization controls can be located at the corners or edges of the array as well as in the middle of the array.

Expression level controls can be probes that hybridize specifically with constitutively expressed genes in the biological sample. Expression level controls can be designed to control for the overall health and metabolic activity of a cell. Examination of the covariance of an expression level control with the expression level of the target nucleic acid can indicate whether measured changes or variations in expression level of a gene is due to changes in transcription rate of that gene or to general variations in health of the cell. Thus, for example, when a cell is in poor health or lacking a critical metabolite the expression levels of both an active target gene and a constitutively expressed gene are expected to decrease. The converse can also be true. Thus where the expression levels of both an expression level control and the target gene appear to both decrease or to both increase, the change can be attributed to changes in the metabolic activity of the cell as a whole, not to differential expression of the target gene in question. Conversely, where the expression levels of the target gene and the expression level control do not covary, the variation in the expression level of the target gene can be attributed to differences in regulation of that gene and not to overall variations in the metabolic activity of the cell.

Virtually any constitutively expressed gene can provide a suitable target for expression level controls. Typically expression level control probes can have sequences complementary to subsequences of constitutively expressed genes including, but not limited to the β-actin gene, the transferrin receptor gene, the GAPDH gene, and the like.

Mismatch controls can also be provided for the probes to the target genes, for expression level controls or for normalization controls. Mismatch controls are typically employed in customized arrays containing probes matched to known mRNA species. For example, some such arrays contain a mismatch probe corresponding to each match probe. The mismatch probe is the same as its corresponding match probe except for at least one position of mismatch. A mismatched base is a base selected so that it is not complementary to the corresponding base in the target sequence to which the probe can otherwise specifically hybridize. One or more mismatches are selected such that under appropriate hybridization conditions (e.g. stringent conditions) the test or control probe can be expected to hybridize with its target sequence, but the mismatch probe cannot hybridize (or can hybridize to a significantly lesser extent). Mismatch probes can contain a central mismatch. Thus, for example, where a probe is a 20 mer, a corresponding mismatch probe can have the identical sequence except for a single base mismatch (e.g., substituting a G, a C or a T for an A) at any of positions 6 through 14 (the central mismatch).

In generic (e.g., random, arbitrary, or haphazard) arrays, since the target nucleic acid(s) are unknown perfect match and mismatch probes cannot be a priori determined, designed, or selected. In this instance, the probes can be provided as pairs where each pair of probes differ in one or more preselected nucleotides. Thus, while it is not known a priori which of the probes in the pair is the perfect match, it is known that when one probe specifically hybridizes to a particular target sequence, the other probe of the pair can act as a mismatch control for that target sequence. The perfect match and mismatch probes need not be provided as pairs, but can be provided as larger collections (e.g., 3, 4, 5, or more) of probes that differ from each other in particular preselected nucleotides.

In both customized and generic arrays mismatch probes can provide a control for non-specific binding or cross-hybridization to a nucleic acid in the sample other than the target to which the probe is complementary. Mismatch probes thus can indicate whether a hybridization is specific or not. For example, if the complementary target is present the perfect match probes can be consistently brighter than the mismatch probes. In addition, if all central mismatches are present, the mismatch probes can be used to detect a mutation. Finally, the difference in intensity between the perfect match and the mismatch probe (I(PM)-I(MM)) can provide a good measure of the concentration of the hybridized material.

Arrays can also include sample preparation/amplification control probes. These can be probes that are complementary to subsequences of control genes selected because they do not normally occur in the nucleic acids of the particular biological sample being assayed. Suitable sample preparation/amplification control probes can include, for example, probes to bacterial genes (e.g., Bio B) where the sample in question is a biological sample from a eukaryote.

The RNA sample can then be spiked with a known amount of the nucleic acid to which the sample preparation/amplification control probe is directed before processing. Quantification of the hybridization of the sample preparation/amplification control probe can then provide a measure of alteration in the abundance of the nucleic acids caused by processing steps (e.g., PCR, reverse transcription, or in vitro transcription).

Quantitation controls can be similar. Typically they can be combined with the sample nucleic acid(s) in known amounts prior to hybridization. They are useful to provide a quantitation reference and permit determination of a standard curve for quantifying hybridization amounts (concentrations).

Methods of Detection

In one method of detection, mRNA or nucleic acid derived therefrom, typically in denatured form, are applied to an array. The component strands of the nucleic acids hybridize to complementary probes, which are identified by detecting label. Optionally, the hybridization signal of matched probes can be compared with that of corresponding mismatched or other control probes. Binding of mismatched probe serves as a measure of background and can be subtracted from binding of matched probes. A significant difference in binding between a perfectly matched probes and a mismatched probes signifies that the nucleic acid to which the matched probes are complementary is present. Binding to the perfectly matched probes is typically at least 1.2, 1.5, 2, 5 or 10 or 20 times higher than binding to the mismatched probes.

In a variation of the above method, nucleic acids are not labeled but are detected by template-directed extension of a probe hybridized to a nucleic acid strand with the nucleic acid strand serving as a template. The probe is extended with a labeled nucleotide, and the position of the label indicates, which probes in the array have been extended. By performing multiple rounds of extension using different bases bearing different labels, it is possible to determine the identity of additional bases in the tag than are determined through complementarity with the probe to which the tag is hybridized. The use of target-dependent extension of probes is described by U.S. Pat. No. 5,547,839, incorporated herein by reference.

In a further variation, probes can be extended with inosine. The inosine strand can be labeled. The addition of degenerate bases, such as inosine (it can pair with all other bases), can increase duplex stability between the polynucleotide probe and the denatured single stranded DNA nucleic acids. The addition of 1-6 inosines onto the end of the probes can increase the signal intensity in both hybridization and ligation reactions on a generic ligation array. This can allow for ligations at higher temperatures. The use of degenerate bases is described in WO 97/27317, incorporated herein by reference.

Ligation reactions can offer improved discriminate between fully complementary hybrids and those that differ by one or more base pairs, particularly in cases where the mismatch is near the 5′ terminus of the polynucleotide probes. Use of a ligation reaction in signal detection increases the stability of the hybrid duplex, improves hybridization specificity (particularly for shorter polynucleotide probes (e.g., 5 to 12-mers), and optionally, provides additional sequence information. Ligation reactions used in signal detection are described in WO 97/27317, incorporated herein by reference. Optionally, ligation reactions can be used in conjunction with template-directed extension of probes, either by inosine or other bases.

Analysis of Hybridization Patterns

The position of label is detected for each probe in the array using a reader, such as described by U.S. Pat. Nos. 5,143,854, 5,578,832, PCT International Application WO 90/15070, each incorporated herein by reference. For customized arrays, the hybridization pattern can then be analyzed to determine the presence and/or relative amounts or absolute amounts of known mRNA species in samples being analyzed as described in e.g., WO 97/10365, incorporated herein by reference. Comparison of the expression patterns of two samples is useful for identifying mRNAs and their corresponding genes that are differentially expressed between the two samples.

The quantitative monitoring of expression levels for large numbers of genes can prove valuable in elucidating gene function, exploring the causes and mechanisms of disease, and for the discovery of potential therapeutic and diagnostic targets. Expression monitoring can be used to monitor the expression (transcription) levels of nucleic acids whose expression is altered in a disease state. For example, inflammation or sepsis can be characterized by the overexpression of a particular marker such as monocyte secretory protein (MCP-1).

Expression monitoring can be used to monitor expression of various genes in response to defined stimuli, such as a drug. This is especially useful in drug research if the end point description is a complex one, not simply asking if one particular gene is overexpressed or underexpressed. Therefore, where a disease state or the mode of action of a drug is not well characterized, the expression monitoring can allow rapid determination of the particularly relevant genes.

In generic arrays, the hybridization pattern is also a measure of the presence and abundance of relative mRNAs in a sample, although it is not immediately known, which probes correspond to which mRNAs in the sample.

However the lack of knowledge regarding the particular genes does not prevent identification of useful therapeutics. For example, if the hybridization pattern on a particular generic array for a healthy cell is known and significantly different from the pattern for a diseased cell, then libraries of compounds can be screened for those that cause the pattern for a diseased cell to become like that for the healthy cell. This provides a detailed measure of the cellular response to a drug.

Generic arrays can also provide a powerful tool for gene discovery and for elucidating mechanisms underlying complex cellular responses to various stimuli. For example, generic arrays can be used for expression fingerprinting. Suppose it is found that the mRNA from a certain cell type displays a distinct overall hybridization pattern that is different under different conditions (e.g., when harboring mutations in particular genes, in a disease state). Then this pattern of expression (an expression fingerprint), if reproducible and clearly differentiable in the different cases can be used as a very detailed diagnostic. It is not required that the pattern be fully interpretable, but just that it is specific for a particular cell state and of diagnostic and/or prognostic relevance.

Both customized and generic arrays can be used in drug safety studies. For example, if one is making a new antibiotic, then it should not significantly affect the expression profile for mammalian cells. The hybridization pattern can be used as a detailed measure of the effect of a drug on cells, for example, as a toxicological screen.

The sequence information provided by the hybridization pattern of a generic array can be used to identify genes encoding mRNAs hybridized to an array. Such methods can be performed using DNA nucleic acids as the target nucleic acids described in WO 97/27317, incorporated herein by reference. DNA nucleic acids can be denatured and then hybridized to the complementary regions of the probes, using standard conditions described in WO 97/27317, incorporated herein by reference. The hybridization pattern indicates which probes are complementary to nucleic acid strands in the sample. Comparison of the hybridization pattern of two samples indicates which probes hybridize to nucleic acid strands that derive from mRNAs that are differentially expressed between the two samples. These probes are of particular interest, because they contain complementary sequence to mRNA species subject to differential expression. The sequence of such probes is known and can be compared with sequences in databases to determine the identity of the full-length mRNAs subject to differential expression provided that such mRNAs have previously been sequenced. Alternatively, the sequences of probes can be used to design hybridization probes or primers for cloning the differentially expressed mRNAs. The differentially expressed mRNAs are typically cloned from the sample in which the mRNA of interest was expressed at the highest level. In some methods, database comparisons or cloning is facilitated by provision of additional sequence information beyond that inferable from probe sequence by template dependent extension as described above.

Screening For Endothelial Cell Activity Modulators

(A) Candidate Bioactive Agents

Having identified a number of suitable expression profiles, the information is used in a wide variety of ways. In one method, the expression profiles can be used in conjunction with high throughput screening techniques, to allow monitoring for expression profile genes after treatment with a candidate agent, Zlokarnik, et al., Science 279: 84-8, 1998, Heid et al., Genome Res. 6: 986, 1996; each incorporated herein by reference. In one method, the candidate agents are added to cells.

The term “candidate bioactive agent” or “drug candidate” or grammatical equivalents as used herein describes any molecule, e.g., protein, oligopeptide, small organic molecule, polysaccharide, polynucleotide, to be tested for bioactive agents that are capable of directly or indirectly altering the activity of an endothelial cell. In one methods, the bioactive agents modulate the expression profiles, or expression profile nucleic acids or proteins provided herein. In a further embodiment of the method, the candidate agents induce an immunosuppressive tolerant response, or maintain such a response as indicated, for example, by the effect of the agent on the expression profile, nucleic acids, proteins or endothelial cell activity as further described below. Generally a plurality of assay mixtures are run in parallel with different agent concentrations to obtain a differential response to the various concentrations. Typically, one of these concentrations serves as a negative control, i.e., at zero concentration or below the level of detection.

Candidate agents encompass numerous chemical classes, though typically they are organic molecules, e.g., small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents can be subjected to directed or random chemical modifications, such as acylation, alkylation, esterification, amidification to produce structural analogs.

In some embodiment, the candidate bioactive agents are proteins. By “protein” herein is meant at least two covalently attached amino acids, which includes proteins, polypeptides, oligopeptides and peptides. The protein can be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the methods herein. “Amino acid” also includes imino acid residues such as proline and hydroxyproline. The side chains can be in either the (R) or the (S) configuration. In further embodiments, the amino acids are in the (S) or L-configuration. If non-naturally occurring side chains are used, non-amino acid substituents can be used, for example to prevent or retard in vivo degradations.

In one method, the candidate bioactive agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and mammalian proteins, and human proteins.

In some methods, the candidate bioactive agents are peptides of from about 5 to about 30 amino acids, typically from about 5 to about 20 amino acids, and typically from about 7 to about 15 being. The peptides can be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they can incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In some methods, the library can be fully randomized, with no sequence preferences or constants at any position. In other methods, the library can be biased. Some positions within the sequence are either held constant, or are selected from a limited number of possibilities. For example, in some methods, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, or to purines. In other methods, the candidate bioactive agents are nucleic acids, as defined above.

As described above generally for proteins, nucleic acid candidate bioactive agents can be naturally occurring nucleic acids, random nucleic acids, or “biased” random nucleic acids. For example, digests of procaryotic or eucaryotic genomes can be used as is outlined above for proteins.

In some methods, the candidate bioactive agents are organic chemical moieties.

(B) Drug Screening Methods

Several different drug screening methods can be accomplished to identify drugs or bioactive agents that modulate endothelial cell activity. One such method is the screening of candidate agents that can induce a particular expression profile, thus generating the associated phenotype. Candidate agents that can mimic or produce an expression profile similar to an immunosuppressive expression profile as shown herein is expected to result in the immunosuppressive phenotype. Similarly, candidate agents that can mimic or produce an expression profile similar to a tolerant expression profile as shown herein is expected to result in the tolerant phenotype. Thus, in some methods, candidate agents can be determined that mimic an expression profile or change one profile to another.

In other methods, after having identified the differentially expressed genes important in any one state, candidate agent screening can be run to alter the expression of individual genes. For example, particularly in the case of target genes whose presence or absence is unique between two states, screening for modulators of the target gene expression can be done.

In other methods, screening can be done to alter the biological function of the expression product of the differentially expressed gene. Again, having identified the importance of a gene in a particular state, screening for agents that bind and/or modulate the biological activity of the gene product can be performed as outlined below.

Thus, screening of candidate agents that modulate endothelial cell activity either at the level of gene expression or protein level can be accomplished.

In some methods, a candidate agent can be administered to endothelial cells in any state, that thus has an associated endothelial cell activity expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e., a peptide) can be put into a viral construct such as a retroviral construct and added to the cell, such that expression of the peptide agent is accomplished; see PCT US97/01019, incorporated herein by reference.

Once the candidate agent has been administered to the cells, the cells can be washed if desired and are allowed to incubate under physiological conditions for some period of time. The cells are then harvested and a new gene expression profile is generated, as outlined herein.

For example, endothelial cells can be screened for agents that produce a tolerant phenotype. A change in at least one gene of the expression profile indicates that the agent has an effect on endothelial cell activity. In one method, an anti-inflammatory profile is induced or maintained, before, during, and/or after stimulation with antigen. By defining such a signature for anti-inflammatory response, screens for new drugs that mimic the anti-inflammatory phenotype can be devised. With this approach, the drug target need not be known and need not be represented in the original expression screening platform, nor does the level of transcript for the target protein need to change. In some methods, the agent induces or maintains a profile which indicates genes downregulated in the presence of APC and upregulated in the presence of thrombin, for example, a combination of at least two genes, including, but not limited to, SH-PTP3, W28170, fructose-6-phosphate,2-kinase/fructose-2,6-bisphosphatase, W28616, BID, NF-KB2, thrombospondin-1,6-phosphofructo-2-kinase/fructose-2,6-bisphosphatase, and neurofibromatosis 2 tumor suppressor.

In some methods, screens can be done on individual genes and gene products. After having identified a particular differentially expressed gene as important in a particular state, screening of modulators of either the expression of the gene or the gene product itself can be completed.

Thus, in some methods, screening for modulators of expression of specific genes can be completed. This will be done as outlined above, but in general the expression of only one or a few genes are evaluated. In some methods, screens are designed to first find candidate agents that can bind to differentially expressed proteins, and then these agents can be used in other assays that evaluate the ability of the candidate agent to modulate differentially expressed activity. There are a number of different assays which can be completed, such as binding assays and activity assays.

In a further method, binding assays are performed. In general, purified or isolated gene product is used; that is, the gene products of one or more differentially expressed nucleic acids are made. Using the nucleic acids of the methods and compositions herein which encode a differentially expressed protein in an endothelial cell state, a variety of expression vectors can be made. The expression vectors can be either self-replicating extrachromosomal vectors or vectors which integrate into a host genome. Generally, these expression vectors include transcriptional and translational regulatory nucleic acid operably linked to the nucleic acid encoding a differentially expressed protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express a differentially expressed protein; for example, transcriptional and translational regulatory nucleic acid sequences from Bacillus are used to express a differentially expressed protein in Bacillus. Numerous types of appropriate expression vectors, and suitable regulatory sequences are known in the art for a variety of host cells.

In general, the transcriptional and translational regulatory sequences can include, but are not limited to, promoter sequences, ribosomal binding sites, transcriptional start and stop sequences, translational start and stop sequences, and enhancer or activator sequences. In one method, the regulatory sequences include a promoter and transcriptional start and stop sequences.

Promoter sequences encode either constitutive or inducible promoters. The promoters can be either naturally occurring promoters or hybrid promoters. Hybrid promoters, which combine elements of more than one promoter, are also known in the art, and are useful in the methods herein.

In addition, the expression vector can comprise additional elements. For example, the expression vector can have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and typically two homologous sequences which flank the expression construct. The integrating vector can be directed to a specific locus in the host cell by selecting the appropriate homologous sequence for inclusion in the vector. Constructs for integrating vectors are well known in the art. Methods to effect homologous recombination are described in PCT US93/03868 and PCT US98/05223, each incorporated herein by reference.

In some methods, the expression vector contains a selectable marker gene to allow the selection of transformed host cells. Selection genes are well known in the art and will vary with the host cell used.

One expression vector system is a retroviral vector system such as is generally described in PCT/US97/01019 and PCT/US97/01048, each incorporated herein by reference.

The differentially expressed proteins of the present methods and compositions are produced by culturing a host cell transformed with an expression vector containing nucleic acid encoding a differentially expressed protein, under the appropriate conditions to induce or cause expression of a differentially expressed protein. The conditions appropriate for differentially expressed protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In some methods, the timing of the harvest is important. For example, the baculoviral systems used in insect cell expression are lytic viruses, and thus harvest time selection can be crucial for product yield.

Appropriate host cells include yeast, bacteria, archebacteria, fungi, and insect and animal cells, including mammalian cells. Of particular interest are Drosophila melangaster cells, Saccharomyces cerevisiae and other yeasts, E. coli, Bacillus subtilis, SF9 cells, C129 cells, 293 cells, Neurospora, BHK, CHO, COS, and HeLa cells. In some methods, endothelial cells are host cells as provided herein, which for example, include non-recombinant cell lines, such as primary cell lines. In addition, purified primary endothelial cells derived from either transgenic or non-transgenic strains can also be used. The host cell can alternatively be an endothelial cell known to have an endothelial cell disorder.

In one method, the differentially expressed proteins are expressed in mammalian cells. Mammalian expression systems can include retroviral systems. A mammalian promoter is any DNA sequence capable of binding mammalian RNA polymerase and initiating the downstream (3′) transcription of a coding sequence for differentially expressed protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenlytion signals include those derived form SV40.

The methods of introducing nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In some methods, differentially expressed proteins are expressed in bacterial systems which are well known in the art.

In other methods, differentially expressed proteins can be produced in insect cells. Expression vectors for the transformation of insect cells, and in particular, baculovirus-based expression vectors, are well known in the art.

In some methods, differentially expressed proteins are produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica.

A differentially expressed protein can also be made as a fusion protein, using techniques well known in the art. For example, for the creation of monoclonal antibodies, if the desired epitope is small, the differentially expressed protein can be fused to a carrier protein to form an immunogen. Alternatively, a differentially expressed protein can be made as a fusion protein to increase expression. For example, when a differentially expressed protein is a differentially expressed peptide, the nucleic acid encoding the peptide can be linked to other nucleic acid for expression purposes. Similarly, differentially expressed proteins of the methods and compositions herein can be linked to protein labels, such as green fluorescent protein (GFP), red fluorescent protein (RFP), yellow fluorescent protein (YFP), and blue fluorescent protein (BFP).

In one embodiment, the proteins are recombinant. A “recombinant protein” is a protein made using recombinant techniques, i.e., through the expression of a recombinant nucleic acid as depicted above. A recombinant protein is distinguished from naturally occurring protein by at least one or more characteristics. For example, the protein can be isolated or purified away from some or all of the proteins and compounds with which it is normally associated in its wild type host, and thus can be substantially pure. For example, an isolated protein is unaccompanied by at least some of the material with which it is normally associated in its natural state, typically constituting at least about 0.5%, typically at least about 5% by weight of the total protein in a given sample. A substantially pure protein comprises at least about 75% by weight of the total protein, at least about 80%, and typically at least about 90%. The definition includes the production of a differentially expressed protein from one organism in a different organism or host cell. Alternatively, the protein can be made at a significantly higher concentration than is normally seen, through the use of a inducible promoter or high expression promoter, such that the protein is made at increased concentration levels. Alternatively, the protein can be in a form not normally found in nature, as in the addition of an epitope tag or amino acid substitutions, insertions and deletions, as discussed below.

In some methods, when the differentially expressed protein is to be used to generate antibodies, the protein must share at least one epitope or determinant with the full length transcription product of the differentially expressed nucleic acids shown herein. By “epitope” or “determinant” herein is meant a portion of a protein which will generate and/or bind an antibody. Thus, in most instances, antibodies made to a smaller protein should be able to bind to the full length protein. In one embodiment, the epitope is unique; that is, antibodies generated to a unique epitope show little or no cross-reactivity.

In some methods, the antibodies provided herein can be capable of reducing or eliminating the biological function of a differentially expressed protein, as is described below. The addition of antibodies (either polyclonal or monoclonal) to the protein (or cells containing the differentially expressed protein) can reduce or eliminate the protein's activity. Generally, at least a 25% decrease in activity is observed, with typically at least about 50% and typically about a 95-100% decrease being observed.

In addition, the proteins can be variant proteins, comprising one more amino acid substitutions, insertions and deletions.

In one method, a differentially expressed protein is purified or isolated after expression. Differentially expressed proteins can be isolated or purified in a variety of ways. Standard purification methods include electrophoretic, molecular, immunological and chromatographic techniques, including ion exchange, hydrophobic, affinity, and reverse-phase HPLC chromatography, and chromatofocusing. For example, a differentially expressed protein can be purified using a standard anti-differentially expressed protein antibody column. Ultrafiltration and diafiltration techniques, in conjunction with protein concentration, are also useful. For general guidance in suitable purification techniques, see Scopes, R., Protein Purification, Springer-Verlag, NY, 1982, incorporated herein by reference. The degree of purification necessary will vary depending on the use of the differentially expressed protein. In some instances no purification will be necessary.

Once the gene product of the differentially expressed gene is made, binding assays can be done. These methods comprise combining a differentially expressed protein and a candidate bioactive agent, and determining the binding of the candidate agent to the differentially expressed protein. Methods utilize a human differentially expressed protein, although other mammalian proteins can also be used, including rodents (mice, rats, hamsters, guinea pigs), farm animals (cows, sheep, pigs, horses) and primates. These latter methods can be used for the development of animal models of human disease. In some methods, variant or derivative differentially expressed proteins can be used, including deletion differentially expressed proteins as outlined above.

The assays herein utilize differentially expressed proteins as defined herein. In some assays, portions of differentially expressed proteins can be utilized. In other assays, portions having differentially expressed activity can be used. In addition, the assays described herein can utilize either isolated differentially expressed proteins or cells comprising the differentially expressed proteins. In some methods, the differentially expressed protein or the candidate agent is non-diffusably bound to an insoluble support having isolated sample receiving areas (e.g., a microtiter plate or an array). The insoluble supports can be made of any composition to which the compositions can be bound, is readily separated from soluble material, and is otherwise compatible with the overall method of screening. The surface of such supports can be solid or porous and of any convenient shape. Examples of suitable insoluble supports include microtiter plates, arrays, membranes and beads. These are typically made of glass, plastic (e.g., polystyrene), polysaccharides, nylon or nitrocellulose, and teflon™. Microtiter plates and arrays are especially convenient because a large number of assays can be carried out simultaneously, using small amounts of reagents and samples. In some cases magnetic beads and the like are included. The particular manner of binding of the composition is not crucial so long as it is compatible with the reagents and overall methods described herein, maintains the activity of the composition and is nondiffusable. Methods of binding include the use of antibodies (which do not sterically block either the ligand binding site or activation sequence when the protein is bound to the support), direct binding to “sticky” or ionic supports, chemical crosslinking, the synthesis of the protein or agent on the surface. Following binding of the protein or agent, excess unbound material is removed by washing. The sample receiving areas can then be blocked through incubation with bovine serum albumin (BSA), casein or other innocuous protein or other moiety. Also included in the methods and compositions herein are screening assays wherein solid supports are not used.

In other methods, the differentially expressed protein is bound to the support, and a candidate bioactive agent is added to the assay. Alternatively, the candidate agent is bound to the support and the differentially expressed protein is added. Novel binding agents include specific antibodies, non-natural binding agents identified in screens of chemical libraries, and peptide analogs. Of particular interest are screening assays for agents that have a low toxicity for human cells. A wide variety of assays can be used for this purpose, including labeled in vitro protein-protein binding assays, electrophoretic mobility shift assays, imunoassays for protein binding, functional assays (such as phosphorylation assays) and the like.

The determination of the binding of the candidate bioactive agent to a differentially expressed protein can be done in a number of ways. In some methods, the candidate bioactive agent is labeled, and binding determined directly. For example, this can be done by attaching all or a portion of a differentially expressed protein to a solid support, adding a labeled candidate agent (for example a fluorescent label), washing off excess reagent, and determining whether the label is present on the solid support. Various blocking and washing steps can be utilized.

By “labeled” herein is meant that the compound is either directly or indirectly labeled with a label which provides a detectable signal, e.g., radioisotope, fluorescers, enzyme, antibodies, particles such as magnetic particles, chemiluminescers, or specific binding molecules. Specific binding molecules include pairs, such as biotin and streptavidin, digoxin and antidigoxin. For the specific binding members, the complementary member would normally be labeled with a molecule which provides for detection, in accordance with known procedures, as outlined above. The label can directly or indirectly provide a detectable signal.

In some methods, only one of the components is labeled. For example, the proteins (or proteinaceous candidate agents) can be labeled at tyrosine positions using ¹²⁵I, or with fluorophores. Alternatively, more than one component can be labeled with different labels; using ¹²⁵I for the proteins, for example, and a fluorophor for the candidate agents.

In other methods, the binding of the candidate bioactive agent is determined through the use of competitive binding assays. In this method, the competitor is a binding moiety known to bind to the target molecule such as an antibody, peptide, binding partner, or ligand. Under certain circumstances, there can be competitive binding as between the bioactive agent and the binding moiety, with the binding moiety displacing the bioactive agent. This assay can be used to determine candidate agents which interfere with binding between differentially expressed proteins and the competitor.

In some methods, the candidate bioactive agent is labeled. Either the candidate bioactive agent, or the competitor, or both, is added first to the protein for a time sufficient to allow binding, if present. Incubations can be performed at any temperature which facilitates optimal activity, typically between about 4° C. and 40° C. Incubation periods are selected for optimum activity, but can also be optimized to facilitate rapid high through put screening. Typically between 0.1 and 1 hour will be sufficient. Excess reagent is generally removed or washed away. The second component is then added, and the presence or absence of the labeled component is followed, to indicate binding.

In other methods, the competitor is added first, followed by the candidate bioactive agent. Displacement of the competitor is an indication that the candidate bioactive agent is binding to the differentially expressed protein and thus is capable of binding to, and potentially modulating, the activity of the differentially expressed protein. In this method, either component can be labeled. For example, if the competitor is labeled, the presence of label in the wash solution indicates displacement by the agent. Alternatively, if the candidate bioactive agent is labeled, the presence of the label on the support indicates displacement.

In other methods, the candidate bioactive agent is added first, with incubation and washing, followed by the competitor. The absence of binding by the competitor can indicate that the bioactive agent is bound to the differentially expressed protein with a higher affinity. Thus, if the candidate bioactive agent is labeled, the presence of the label on the support, coupled with a lack of competitor binding, can indicate that the candidate agent is capable of binding to the differentially expressed protein.

Competitive binding methods can also be run as differential screens. These methods can comprise a differentially expressed protein and a competitor in a first sample. A second sample comprises a candidate bioactive agent, a differentially expressed protein and a competitor. The binding of the competitor is determined for both samples, and a change, or difference in binding between the two samples indicates the presence of an agent capable of binding to the differentially expressed protein and potentially modulating its activity. If the binding of the competitor is different in the second sample relative to the first sample, the agent is capable of binding to the differentially expressed protein.

Other methods utilize differential screening to identify drug candidates that bind to the native differentially expressed protein, but cannot bind to modified differentially expressed proteins. The structure of the differentially expressed protein can be modeled, and used in rational drug design to synthesize agents that interact with that site. Drug candidates that affect differentially expressed bioactivity are also identified by screening drugs for the ability to either enhance or reduce the activity of the protein.

In some methods, screening for agents that modulate the activity of differentially expressed proteins are performed. In general, this will be done on the basis of the known biological activity of the differentially expressed protein. In these methods, a candidate bioactive agent is added to a sample of the differentially expressed protein, as above, and an alteration in the biological activity of the protein is determined. “Modulating the activity” includes an increase in activity, a decrease in activity, or a change in the type or kind of activity present. Thus, in these methods, the candidate agent should both bind to differentially expressed (although this may not be necessary), and alter its biological or biochemical activity as defined herein. The methods include both in vitro screening methods, as are generally outlined above, and in vivo screening of cells for alterations in the presence, distribution, activity or amount of the differentially expressed protein.

Some methods comprise combining a differentially expressed sample and a candidate bioactive agent, then evaluating the effect on endothelial cell anti-inflammatory activity. By “differentially expressed activity” or grammatical equivalents herein is meant one of endothelial cell biological activities, including, but not limited to, its ability to affect suppression, tolerance and activation. One activity herein is the capability to bind to a target gene, or modulate an expression profile. Expression profiles are induced or maintained and/or the desired endothelial cell state is induced or maintained.

In other methods, the activity of the differentially expressed protein is increased; in other methods, the activity of the differentially expressed protein is decreased. Thus, bioactive agents that are antagonists are useful in some methods, and bioactive agents that are agonists are useful in other methods.

Methods for screening for bioactive agents capable of modulating the activity of a differentially expressed protein are provided. These methods comprise adding a candidate bioactive agent, as defined above, to a cell comprising differentially expressed proteins. Cell types include almost any cell. The cells contain a recombinant nucleic acid that encodes a differentially expressed protein. In one method, a library of candidate agents are tested on a plurality of cells. The effect of the candidate agent on endothelial cell activity is then evaluated.

Positive controls and negative controls can be used in the assays. All control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples can be counted in a scintillation counter to determine the amount of bound compound.

A variety of other reagents can be included in the screening assays. These include reagents like salts, neutral proteins (e.g., albumin and detergents) which can be used to facilitate optimal protein-protein binding and/or reduce non-specific or background interactions. Reagents that otherwise improve the efficiency of the assay, (such as protease inhibitors, nuclease inhibitors, anti-microbial agents) can also be used. The mixture of components can be added in any order that provides for the requisite binding.

The components provided herein for the assays provided herein can also be combined to form kits. The kits can be based on the use of the protein and/or the nucleic acid encoding the differentially expressed proteins. Assays regarding the use of nucleic acids are further described below.

(C) Animal Models

In one method, nucleic acids which encode differentially expressed proteins or their modified forms can also be used to generate either transgenic animals, including “knock-in” and “knock out” animals which, in turn, are useful in the development and screening of therapeutically useful reagents. A non-human transgenic animal (e.g., a mouse or rat) is an animal having cells that contain a transgene, which transgene is introduced into the animal or an ancestor of the animal at a prenatal, e.g., an embryonic stage. A transgene is a DNA which is integrated into the genome of a cell from which a transgenic animal develops, and can include both the addition of all or part of a gene or the deletion of all or part of a gene. In some methods, cDNA encoding a differentially expressed protein can be used to clone genomic DNA encoding a differentially expressed protein in accordance with established techniques and the genomic sequences used to generate transgenic animals that contain cells which either express (or overexpress) or suppress the desired DNA. Methods for generating transgenic animals, particularly animals such as mice or rats, have become conventional in the art and are described, for example, in U.S. Pat. Nos. 4,736,866 and 4,870,009, each incorporated herein by reference. Typically, particular cells would be targeted for a differentially expressed protein transgene incorporation with tissue-specific enhancers. Transgenic animals that include a copy of a transgene encoding a differentially expressed protein introduced into the germ line of the animal at an embryonic stage can be used to examine the effect of increased expression of the desired nucleic acid. Such animals can be used as tester animals for reagents thought to confer protection from, for example, pathological conditions associated with its overexpression. In accordance with this facet, an animal is treated with the reagent and a reduced incidence of the pathological condition, compared to untreated animals bearing the transgene, would indicate a potential therapeutic intervention for the pathological condition. Similarly, non-human homologues of a differentially expressed protein can be used to construct a transgenic animal comprising a differentially expressed protein “knock out” animal which has a defective or altered gene encoding a differentially expressed protein as a result of homologous recombination between the endogenous gene encoding a differentially expressed protein and altered genomic DNA encoding a differentially expressed protein introduced into an embryonic cell of the animal. For example, cDNA encoding a differentially expressed protein can be used to clone genomic DNA encoding a differentially expressed protein in accordance with established techniques. A portion of the genomic DNA encoding a differentially expressed protein can be deleted or replaced with another gene, such as a gene encoding a selectable marker which can be used to monitor integration. Typically, several kilobases of unaltered flanking DNA (both at the 5′ and 3′ ends) are included in the vector (see, e.g., Thomas and Capecchi, Cell 51: 503, 1987, incorporated herein by reference for a description of homologous recombination vectors). The vector is introduced into an embryonic stem cell line (e.g., by electroporation) and cells in which the introduced DNA has homologously recombined with the endogenous DNA are selected (see, e.g., Li et al., Cell 69: 915, 1992; incorporated herein by reference). The selected cells are then injected into a blastocyst of an animal (e.g., a mouse or rat) to form aggregation chimeras (see, e.g., Bradley, in Teratocarcinomas and Embryonic Stem Cells: A Practical Approach, E. J. Robertson, ed. (IRL, Oxford, 1987), pp. 113-152). A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of a differentially expressed protein polypeptide.

Animal models for endothelial cell related disorders, or having a particular state of endothelial cell activity can include, for example, genetic models. For example, such animal models can include the nonobese diabetic (NOD) mouse (see, e.g., McDuffie, M., Curr Opin Immunol. 10(6): 704-9, 1998; Tochino, Y., Crit Rev Immunol 8(1): 49-81, 1987), and experimental autoimmune encephalomyelitis (EAE) (see, e.g., Wong, F. S., Immunol Rev 169: 93-104, 1999). See also Schwartz, R. S. and Datta, S. K., Autoimmunity and Autoimmune Diseases, Ch. 31, in Fundamental Immunology, Paul, W. E. (ed.) (Raven Press 1989), each incorporated herein by reference. Other models can include studies involving transplant rejection.

Animal models exhibiting endothelial cell related disorder-like symptoms can be engineered by utilizing, for example, differentially expressed sequences in conjunction with techniques for producing transgenic animals that are well known to those of skill in the art. For example, gene sequences can be introduced into, and overexpressed in, the genome of the animal of interest, or, if endogenous target gene sequences are present, they can either be overexpressed or, alternatively, can be disrupted in order to underexpress or inactivate target gene expression.

In order to overexpress a target gene sequence, the coding portion of the target gene sequence can be ligated to a regulatory sequence which is capable of driving gene expression in the animal and cell type of interest. Such regulatory regions will be well known to those of skill in the art, and can be utilized in the absence of undue experimentation.

For underexpression of an endogenous target gene sequence, such a sequence can be isolated and engineered such that when reintroduced into the genome of the animal of interest, the endogenous target gene alleles will be inactivated. The engineered target gene sequence is introduced via gene targeting such that the endogenous target sequence is disrupted upon integration of the engineered target sequence into the animal's genome.

Animals of any species, including, but not limited to, mice, rats, rabbits, guinea pigs, pigs, micro-pigs, goats, and non-human primates, e.g., baboons, monkeys, and chimpanzees can be used to generate animal models of endothelial cell related disorders or being a perpetually desired state of the endothelial cell.

(D) Nucleic Acid Based Therapeutics

Nucleic acids encoding differentially expressed polypeptides, antagonists or agonists can also be used in gene therapy. Broadly speaking, a gene therapy vector is an exogenous polynucleotide which produces a medically useful phenotypic effect upon the mammalian cell(s) into which it is transferred. A vector can or can not have an origin of replication. For example, it is useful to include an origin of replication in a vector for propagation of the vector prior to administration to a patient. However, the origin of replication can often be removed before administration if the vector is designed to integrate into host chromosomal DNA or bind to host mRNA or DNA. Vectors used in gene therapy can be viral or nonviral. Viral vectors are usually introduced into a patient as components of a virus. Nonviral vectors, typically dsDNA, can be transferred as naked DNA or associated with a transfer-enhancing vehicle, such as a receptor-recognition protein, lipoamine, or cationic lipid.

Viral vectors, such as retroviruses, adenoviruses, adenoassociated viruses and herpes viruses, are often made up of two components, a modified viral genome and a coat structure surrounding it (see generally Smith et al., Ann. Rev. Microbiol. 49: 807-838, 1995, incorporated herein by reference), although sometimes viral vectors are introduced in naked form or coated with proteins other than viral proteins. Most current vectors have coat structures similar to a wildtype virus. This structure packages and protects the viral nucleic acid and provides the means to bind and enter target cells. However, the viral nucleic acid in a vector designed for gene therapy is changed in many ways. The goals of these changes are to disable growth of the virus in target cells while maintaining its ability to grow in vector form in available packaging or helper cells, to provide space within the viral genome for insertion of exogenous DNA sequences, and to incorporate new sequences that encode and enable appropriate expression of the gene of interest. Thus, vector nucleic acids generally comprise two components: essential cis-acting viral sequences for replication and packaging in a helper line and the transcription unit for the exogenous gene. Other viral functions are expressed in trans in a specific packaging or helper cell line.

Nonviral nucleic acid vectors used in gene therapy include plasmids, RNAs, antisense oligonucleotides (e.g., methylphosphonate or phosphorothiolate), polyamide nucleic acids, and yeast artificial chromosomes (YACs). Such vectors typically include an expression cassette for expressing a protein or RNA. The promoter in such an expression cassette can be constitutive, cell type-specific, stage-specific, and/or modulatable (e.g., by hormones such as glucocorticoids; MMTV promoter). Transcription can be increased by inserting an enhancer sequence into the vector. Enhancers are cis-acting sequences of between 10 to 300 bp that increase transcription by a promoter. Enhancers can effectively increase transcription when either 5′ or 3′ to the transcription unit. They are also effective if located within an intron or within the coding sequence itself. Typically, viral enhancers are used, including SV40 enhancers, cytomegalovirus enhancers, polyoma enhancers, and adenovirus enhancers. Enhancer sequences from mammalian systems are also commonly used, such as the mouse immunoglobulin heavy chain enhancer.

Gene therapy vectors can be delivered in vivo by administration to an individual patient, typically by systemic administration (e.g., intravenous, intraperitoneal, intramuscular, subdermal, or intracranial infusion) or topical application. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Modulating Signaling in Endothelial Cells Via the PAR1 Pathway

(A) Assays for Modulators of PAR1 Signaling

In numerous embodiments of this invention, the level of PAR1 signaling will be modulated in a cell by administering to the cell, in vivo or in vitro, any of a large number of PAR1-modulating molecules, e.g., polypeptides, antibodies, amino acids, nucleotides, lipids, carbohydrates, or any organic or inorganic molecule.

To identify molecules capable of modulating PAR1, assays will be performed to detect the effect of various compounds on PAR1 signaling activity in a cell. PAR1 signaling can be assessed using a variety of in vitro and in vivo assays to determine functional, chemical, and physical effects, e.g., measuring the binding of PAR1 to other molecules (e.g., radioactive binding to APC or EPCR), measuring protein and/or RNA levels of APC/EPCR/PAR1 induced genes that protect against an inflammatory response, or measuring other aspects of APC/EPCR/PAR1 induced protective genes, e.g., phosphorylation levels, transcription levels, receptor activity, ligand binding and the like. Such assays can be used to test for both activators and inhibitors of PAR1 signaling. Modulators thus identified are useful for, e.g., many diagnostic and therapeutic applications.

The APC/EPCR/PAR1 induced protective protein of the assay will typically be a recombinant or naturally occurring polypeptide or a conservatively modified variant thereof. Alternatively, the APC/EPCR/PAR1 induced protective protein of the assay will be derived from a eukaryote and include an amino acid subsequence having amino acid sequence identity to the naturally occurring APC/EPCR/PAR1 induced protective protein. Generally, the amino acid sequence identity will be at least 70%, optionally at least 75%, 85%, or 86%, 87%, 88%, 89%, 90%, 91%, 92%, 93%, 94%, 95%, 96%, 97%, 98% or greater. Optionally, the polypeptide of the assays will comprise a domain of an APC/EPCR/PAR1 induced protective protein. In certain embodiments, a domain of an APC/EPCR/PAR1 induced protective protein, e.g., a zinc finger binding domain, is bound to a solid substrate and used, e.g., to isolate any molecules that can bind to and/or modulate their activity. In certain embodiments, a domain of an APC/EPCR/PAR1 induced protective polypeptide, e.g., an N-terminal domain, a C-terminal domain, is fused to a heterologous polypeptide, thereby forming a chimeric polypeptide. Such chimeric polypeptides are also useful, e.g., in assays to identify modulators of an APC/EPCR/PAR1 induced protective protein.

Samples or assays that are treated with a potential APC/EPCR/PAR1 induced protective protein inhibitor or activator are compared to control samples without the test compound, to examine the extent of modulation. Control samples (untreated with activators or inhibitors) are assigned a relative APC/EPCR/PAR1 induced protective protein activity value of 100. Inhibition of an APC/EPCR/PAR1 induced protective protein is achieved when the APC/EPCR/PAR1 induced protective protein activity value relative to the control is about 90%, optionally about 50%, optionally about 25-0%. Activation of a APC/EPCR/PAR1 induced protective protein is achieved when the APC/EPCR/PAR1 induced protective protein activity value relative to the control is about 110%, optionally about 150%, 200-500%, or about 1000-2000%.

The effects of the test compounds upon the function of the polypeptides can be measured by examining any of the parameters described above. Any suitable physiological change that affects APC/EPCR/PAR1 induced protective protein activity can be used to assess the influence of a test compound on the polypeptides of this invention. When the functional consequences are determined using intact cells or animals, one can also measure a variety of effects such as changes in cell growth or changes in cell-cell interactions.

Modulators of APC/EPCR/PAR1 induced protective protein that act by modulating APC/EPCR/PAR1 induced protective protein gene expression can also be identified. For example, a host cell containing a APC/EPCR/PAR1 induced protective protein of interest is contacted with a test compound for a sufficient time to effect any interactions, and then the level of gene expression is measured. The amount of time to effect such interactions can be empirically determined, such as by running a time course and measuring the level of transcription as a function of time. The amount of transcription can be measured using any method known to those of skill in the art to be suitable. For example, mRNA expression of the protein of interest can be detected using Northern blots or by detecting their polypeptide products using immunoassays.

(B) Assays for APC/EPCR/PAR1 Induced Protective Protein—Interacting Compounds

In certain embodiments, assays will be performed to identify molecules that physically interact with APC/EPCR/PAR1 induced protective proteins. Such molecules can be any type of molecule, including polypeptides, polynucleotides, amino acids, nucleotides, carbohydrates, lipids, or any other organic or inorganic molecule. Such molecules can represent molecules that normally interact with APC/EPCR/PAR1 induced protective protein or can be synthetic or other molecules that are capable of interacting with APC/EPCR/PAR1 induced protective protein and that can potentially be used as lead compounds to identify classes of molecules that can interact with and/or modulate APC/EPCR/PAR1 induced protective protein. Such assays can represent physical binding assays, such as affinity chromatography, immunoprecipitation, two-hybrid screens, or other binding assays, or can represent genetic assays.

In any of the binding or functional assays described herein, in vivo or in vitro, any APC/EPCR/PAR1 induced protective protein, or any derivative, variation, homolog, or fragment of a naturally occurring APC/EPCR/PAR1 induced protective protein, can be used. Preferably, the APC/EPCR/PAR1 induced protective protein has at least about 85% identity to the amino acid sequence of the naturally occurring APC/EPCR/PAR1 induced protective protein. In numerous embodiments, a fragment of a APC/EPCR/PAR1 induced protective protein is used. Such fragments can be used alone, in combination with other APC/EPCR/PAR1 induced protective protein fragments, or in combination with sequences from heterologous proteins, e.g., the fragments can be fused to a heterologous polypeptides, thereby forming a chimeric polypeptide.

Compounds that interact with APC/EPCR/PAR1 induced protective proteins can be isolated based on an ability to specifically bind to a APC/EPCR/PAR1 induced protective protein or fragment thereof. In numerous embodiments, the APC/EPCR/PAR1 induced protective protein or protein fragment will be attached to a solid support. In one embodiment, affinity columns are made using the APC/EPCR/PAR1 induced protective polypeptide, and physically-interacting molecules are identified. It will be apparent to one of skill that chromatographic techniques can be performed at any scale and using equipment from many different manufactures (e.g., Pharmacia Biotechnology). In addition, molecules that interact with APC/EPCR/PAR1 induced protective proteins in vivo can be identified by co-immunoprecipitation or other methods, i.e., immunoprecipitating APC/EPCR/PAR1 induced protective protein using anti-APC/EPCR/PAR1 induced protective protein antibodies from a cell or cell extract, and identifying compounds, e.g., proteins, that are precipitated along with the APC/EPCR/PAR1 induced protective protein. Such methods are well known to those of skill in the art and are taught, e.g., in Ausubel et al., Sambrook et al., and Harlow & Lane, all supra.

(C) Increasing APC/EPCR/PAR1 Induced Protective Protein Activity Levels in Cells

In certain embodiments, this invention provides methods of treating inflammation or sepsis by increasing APC/EPCR/PAR1 induced protective protein levels in a cell. Typically, such methods are used to increase a reduced level of APC/EPCR/PAR1 induced protective protein, e.g., a reduced level in an endothelial cell, and can be performed in any of a number of ways, e.g., increasing the copy number of APC/EPCR/PAR1 induced protective genes or increasing the level of APC/EPCR/PAR1 induced protective mRNA, protein, or protein activity in a cell. Preferably, the level of APC/EPCR/PAR1 induced protective protein activity is increased to a level typical of a normal, endothelial cell, but the level can be increased to any level that is sufficient to increase PAR1 signaling in the endothelial cell, including to levels above or below those typical of normal cells. Preferably, such methods involve the use of activators of APC/EPCR/PAR1 induced protective protein, where an “activator of APC/EPCR/PAR1 induced protective protein” is a molecule that acts to increase APC/EPCR/PAR1 induced protective gene polynucleotide levels, polypeptide levels and/or protein activity. Such activators can include, but are not limited to, small molecule activators of APC/EPCR/PAR1 induced protective protein.

(D) Reducing APC/EPCR/PAR1 Induced Protective Protein Activity Levels In Cells

In certain embodiments, this invention provides methods of treating inflammation or sepsis by reducing APC/EPCR/PAR1 induced protective protein levels in a cell. Typically, such methods are used to reduce an elevated level of APC/EPCR/PAR1 induced protective protein, e.g., an elevated level in an endothelial cell, and can be performed in any of a number of ways, e.g., lowering the copy number of APC/EPCR/PAR1 induced protective protein genes or decreasing the level of mRNA, protein, or protein activity in a cell. Preferably, the level of APC/EPCR/PAR1 induced protective protein activity is lowered to a level typical of a normal endothelial cell, but the level can be reduced to any level that is sufficient to increase PAR1 signaling of the cell, including to levels above or below those typical of normal cells. Preferably, such methods involve the use of inhibitors of APC/EPCR/PAR1 induced protective protein, where an “inhibitor of APC/EPCR/PAR1 induced protective protein” is a molecule that acts to reduce APC/EPCR/PAR1 induced protective protein polynucleotide levels, polypeptide levels and/or protein activity. Such inhibitor s include, but are not limited to, antisense polynucleotides, ribozymes, antibodies, dominant negative APC/EPCR/PAR1 induced protective protein forms, and small molecule inhibitors of APC/EPCR/PAR1 induced protective protein.

In preferred embodiments, APC/EPCR/PAR1 induced protective protein levels will be reduced so as to reduce inflammation or sepsis with elevated APC/EPCR/PAR1 induced protective protein levels. The proliferation of a cell refers to the rate at which the cell or population of cells divides, or to the extent to which the cell or population of cells divides or increases in number. Proliferation can reflect any of a number of factors, including the rate of cell growth and division and the rate of cell death. Without being bound by the following offered theory, it is suggested that the amplification and/or overexpression of the APC/EPCR/PAR1 induced gene in endothelial cells to prevent proinflammatory signaling. The anti-inflammatory activity of APC/EPCR/PAR1 induced protective protein can act to prevent coagulant and inflammatory exacerbation in sepsis. The ability of any of the present compounds to affect APC/EPCR/PAR1 induced protective protein activity can be determined based on any of a number of factors, including, but not limited to, a level of APC/EPCR/PAR1 induced protective polynucleotide, e.g., mRNA or gDNA, the level of APC/EPCR/PAR1 induced protective polypeptide, the degree of binding of a compound to a APC/EPCR/PAR1 induced protective polynucleotide or polypeptide, APC/EPCR/PAR1 induced protective protein intracellular localization, or any functional properties of APC/EPCR/PAR1 induced protective protein, such as the ability of APC/EPCR/PAR1 induced protective protein activity to prevent coagulant and inflammatory exacerbation in sepsis.

(E) Inhibitors of APC/EPCR/PAR1 Induced Protective Polynucleotides

In certain embodiments, APC/EPCR/PAR1 induced protective protein activity is downregulated, or entirely inhibited, by the use of antisense polynucleotide, i.e., a nucleic acid complementary to, and which can preferably hybridize specifically to, a coding mRNA nucleic acid sequence, e.g, APC/EPCR/PAR1 induced mRNA, or a subsequence thereof. Binding of the antisense polynucleotide to the mRNA reduces the translation and/or stability of the APC/EPCR/PAR1 induced mRNA.

In the context of this invention, antisense polynucleotides can comprise naturally-occurring nucleotides, or synthetic species formed from naturally-occurring subunits or their close homologs. Antisense polynucleotides can also have altered sugar moieties or inter-sugar linkages. Exemplary among these are the phosphorothioate and other sulfur containing species which are known for use in the art. All such analogs are comprehended by this invention so long as they function effectively to hybridize with APC/EPCR/PAR1 induced mRNA.

Such antisense polynucleotides can readily be synthesized using recombinant means, or can be synthesized in vitro. Equipment for such synthesis is sold-by several vendors, including Applied Biosystems. The preparation of other oligonucleotides such as phosphorothioates and alkylated derivatives is also well known to those of skill in the art.

In addition to antisense polynucleotides, ribozymes can be used to target and inhibit transcription of APC/EPCR/PAR1 induced protective protein. A ribozyme is an RNA molecule that catalytically cleaves other RNA molecules. Different kinds of ribozymes have been described, including group I ribozymes, hammerhead ribozymes, hairpin ribozymes, RNAse P, and axhead ribozymes (see, e.g., Castanotto et al., Adv. in Pharmacology 25: 289-317, 1994 for a general review of the properties of different ribozymes).

The general features of hairpin ribozymes are described, e.g., in Hampel et al., Nucl. Acids Res., 18: 299-304, 1990; Hampel et al., 1990, European Patent Publication No. 0 360 257; U.S. Pat. No. 5,254,678. Methods of preparing are well known to those of skill in the art (see, e.g., Wong-Staal et al., WO 94/26877; Ojwang et al., Proc. Natl. Acad. Sci. USA, 90: 6340-6344, 1993; Yamada et al., Human Gene Therapy 1: 39-45, 1994; Leavitt et al., Proc. Natl. Acad. Sci. USA, 92: 699-703, 1995; Leavitt et al., Human Gene Therapy 5: 1151-120, 1994; and Yamada et al., Virology 205: 121-126, 1994).

APC/EPCR/PAR1 induced protective protein activity can also be decreased by the addition of an inhibitor of the APC/EPCR/PAR1 induced protective protein. This can be accomplished in any of a number of ways, including by providing a dominant negative APC/EPCR/PAR1 induced protective polypeptide, e.g., a form of APC/EPCR/PAR1 induced protective protein that itself has no activity and which, when present in the same cell as a functional APC/EPCR/PAR1 induced protective protein, reduces or eliminates the APC/EPCR/PAR1 induced protective protein activity of the functional APC/EPCR/PAR1 induced protective protein. Design of dominant negative forms is well known to those of skill and is described, e.g., in Herskowitz, Nature, 329: 219-22, 1987. Also, inactive polypeptide variants (muteins) can be used, e.g., by screening for the ability to inhibit APC/EPCR/PAR1 induced protective protein activity. Methods of making muteins are well known to those of skill (see, e.g., U.S. Pat. Nos. 5,486,463, 5,422,260, 5,116,943, 4,752,585, 4,518,504). In addition, any small molecule, e.g., any peptide, amino acid, nucleotide, lipid, carbohydrate, or any other organic or inorganic molecule can be screened for the ability to bind to or inhibit APC/EPCR/PAR1 induced protective protein activity, as described below.

(F) Modulators and Binding Compounds

The compounds tested as modulators of a APC/EPCR/PAR1 induced protective protein can be any small chemical compound, or a biological entity, such as a protein, sugar, nucleic acid or lipid. Typically, test compounds will be small chemical molecules and peptides. Essentially any chemical compound can be used as a potential modulator or binding compound in the assays of the invention, although most often compounds can be dissolved in aqueous or organic (especially DMSO-based) solutions. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In one preferred embodiment, high throughput screening methods involve providing a combinatorial chemical or peptide library containing a large number of potential therapeutic compounds (potential modulator or binding compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka, Int. J. Pept. Prot. Res. 37: 487-493, 1991 and Houghton et al., Nature 354: 84-88, 1991). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90: 6909-6913, 1993), vinylogous polypeptides (Hagihara et al., J. Amer. Chem. Soc. 114: 6568, 1992), nonpeptidal peptidomimetics with glucose scaffolding (Hirschmann et al., J. Amer. Chem. Soc. 114: 9217-9218, 1992), analogous organic syntheses of small compound libraries (Chen et al., J. Amer. Chem. Soc. 116: 2661, 1994), oligocarbamates (Cho et al., Science 261: 1303, 1993), and/or peptidyl phosphonates (Campbell et al., J. Org. Chem. 59: 658, 1994), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14: 309-314, 1996 and PCT/US96/10287), carbohydrate libraries (see, e.g., Liang et al., Science, 274: 1520-1522, 1996 and U.S. Pat. No. 5,593,853), small organic molecule libraries (see, e.g., benzodiazepines, Baum, C&EN, page 33, Jan. 18, 1993; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

(G) Solid State and Soluble High Throughput Assays

In one embodiment, the invention provides soluble assays using molecules such as an N-terminal or C-terminal domain either alone or covalently linked to a heterologous protein to create a chimeric molecule. In another embodiment, the invention provides solid phase based in vitro assays in a high throughput format, where a domain, chimeric molecule, APC/EPCR/PAR1 induced protective protein, or cell or tissue expressing a APC/EPCR/PAR1 induced protective protein is attached to a solid phase substrate.

In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 different compounds is possible using the integrated systems of the invention. More recently, microfluidic approaches to reagent manipulation have been developed.

The molecule of interest can be bound to the solid state component, directly or indirectly, via covalent or non covalent linkage, e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85: 2149-2154, 1993 (describing solid phase synthesis of, e.g., peptides); Geysen et al., J. Immun. Meth. 102: 259-274, 1987 (describing synthesis of solid phase components on pins); Frank & Doring, Tetrahedron 44: 6031-6040, 1988 (describing synthesis of various peptide sequences on cellulose disks); Fodor et al., Science, 251: 767-777, 1991; Sheldon et al., Clinical Chemistry 39: 718-719, 1993; and Kozal et al., Nature Medicine 2: 753-759, 1996 (all describing arrays of biopolymers fixed to solid substrates). Nonchemical approaches for fixing tag binders to substrates include other common methods, such as heat, cross-linking by UV radiation, and the like.

(H) Rational Drug Design Assays

Yet another assay for compounds that modulate APC/EPCR/PAR1 induced protective protein activity involves computer assisted drug design, in which a computer system is used to generate a three-dimensional structure of a APC/EPCR/PAR1 induced protective protein based on the structural information encoded by its amino acid sequence. The input amino acid sequence interacts directly and actively with a pre-established algorithm in a computer program to yield secondary, tertiary, and quaternary structural models of the protein. The models of the protein structure are then examined to identify regions of the structure that have the ability to bind. These regions are then used to identify compounds that bind to the protein.

The three-dimensional structural model of the protein is generated by entering protein amino acid sequences of at least 10 amino acid residues or corresponding nucleic acid sequences encoding a APC/EPCR/PAR1 induced protective polypeptide into the computer system. The nucleotide sequence encoding the polypeptide, or the amino acid sequence thereof, and conservatively modified versions thereof, of the naturally occurring gene sequence. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential modulator binding regions are identified by the computer system. Three-dimensional structures for potential modulators are generated by entering amino acid or nucleotide sequences or chemical formulas of compounds, as described above. The three-dimensional structure of the potential modulator is then compared to that of the APC/EPCR/PAR1 induced protective protein to identify compounds that bind to the protein. Binding affinity between the protein and compound is determined using energy terms to determine which compounds have an enhanced probability of binding to the protein.

Computer systems are also used to screen for mutations, polymorphic variants, alleles and interspecies homologs of APC/EPCR/PAR1 induced genes. Such mutations can be associated with disease states or genetic traits. As described above, GeneChip™ and related technology can also be used to screen for mutations, polymorphic variants, alleles and interspecies homologs. Once the variants are identified, diagnostic assays can be used to identify patients having such mutated genes. Identification of the mutated APC/EPCR/PAR1 induced genes involves receiving input of a first nucleic acid or amino acid sequence of the naturally occurring APC/EPCR/PAR1 induced gene, respectively, and conservatively modified versions thereof. The sequence is entered into the computer system as described above. The first nucleic acid or amino acid sequence is then compared to a second nucleic acid or amino acid sequence that has substantial identity to the first sequence. The second sequence is entered into the computer system in the manner described above. Once the first and second sequences are compared, nucleotide or amino acid differences between the sequences are identified. Such sequences can represent allelic differences in various APC/EPCR/PAR1 induced genes, and mutations associated with disease states and genetic traits.

Diagnostic Methods

In addition to assays, the creation of animal models, and nucleic acid based therapeutics, identification of important differentially expressed genes allows the use of these genes in diagnosis (e.g., diagnosis of cell states and abnormal endothelial cell conditions). Disorders based on mutant or variant differentially expressed genes can be determined. Methods for identifying cells containing variant differentially expressed genes comprising determining all or part of the sequence of at least one endogeneous differentially expressed genes in a cell are provided. As will be appreciated by those in the art, this can be done using any number of sequencing techniques. Methods of identifying the differentially expressed genotype of an individual comprising determining all or part of the sequence of at least one differentially expressed gene of the individual are also provided. This is generally done in at least one tissue of the individual, and can include the evaluation of a number of tissues or different samples of the same tissue. The method can include comparing the sequence of the sequenced differentially expressed gene to a known differentially expressed gene, i.e., a wild-type gene.

The sequence of all or part of the differentially expressed gene can then be compared to the sequence of a known differentially expressed gene to determine if any differences exist. This can be done using any number of known sequence identity programs, such as Bestfit, and others outlined herein. In some methods, the presence of a difference in the sequence between the differentially expressed gene of the patient and the known differentially expressed gene is indicative of a disease state or a propensity for a disease state, as outlined herein.

Similarly, diagnosis of endothelial cell states can be done using the methods and compositions herein. By evaluating the gene expression profile of endothelial cells from a patient, the endothelial cell state can be determined. This is particularly useful to verify the action of a drug, for example an immunosuppressive drug. Other methods comprise administering the drug to a patient and removing a cell sample, particularly of endothelial cells, from the patient. The gene expression profile of the cell is then evaluated, as outlined herein, for example by comparing it to the expression profile from an equivalent sample from a healthy individual. In this manner, both the efficacy (i.e., whether the correct expression profile is being generated from the drug) and the dose (is the dosage correct to result in the correct expression profile) can be verified.

The present discovery relating to the role of differentially expressed in endothelial cells thus provides methods for inducing or maintaining differing endothelial cell states. In one method, the differentially expressed proteins, and particularly differentially expressed fragments, are useful in the study or treatment of conditions which are mediated by endothelial cell activity, i.e., to diagnose, treat or prevent endothelial cell-mediated disorders. Thus, “endothelial cell mediated disorders” or “disease states” can include conditions involving, for example, arthritis, diabetes, or multiple sclerosis.

Methods of modulating endothelial cell activity in cells or organisms are provided. Some methods comprise administering to a cell an anti-differentially expressed antibody or other agent identified herein or by the methods provided herein, that reduces or eliminates the biological activity of the endogeneous differentially expressed protein. Alternatively, the methods comprise administering to a cell or organism a recombinant nucleic acid encoding a differentially expressed protein or modulator including anti-sense nucleic acids. As will be appreciated by those in the art, this can be accomplished in any number of ways. In some methods, the activity of differentially expressed is increased by increasing the amount of differentially expressed in the cell, for example by overexpressing the endogeneous differentially expressed or by administering a differentially expressed gene, using known gene therapy techniques, for example. In one method, the gene therapy techniques include the incorporation of the exogenous gene using enhanced homologous recombination (EHR), for example as described in PCT/US93/03868, hereby incorporated by reference in its entirety.

Methods for diagnosing an endothelial cell activity related condition in an individual are provided. The methods comprise measuring the activity of differentially expressed protein in a tissue from the individual or patient, which can include a measurement of the amount or specific activity of the protein. This activity is compared to the activity of differentially expressed from either an unaffected second individual or from an unaffected tissue from the first individual. When these activities are different, the first individual can be at risk for an endothelial cell activity mediated disorder.

Furthermore, nucleotide sequences encoding a differentially expressed protein can also be used to construct hybridization probes for mapping the gene which encodes that differentially expressed protein and for the genetic analysis of individuals with genetic disorders. The nucleotide sequences provided herein can be mapped to a chromosome and specific regions of a chromosome using known techniques, such as in situ hybridization, linkage analysis against known chromosomal markers, and hybridization screening with libraries.

Antibodies

In some methods, the differentially expressed proteins can be used to generate polyclonal and monoclonal antibodies to differentially expressed proteins, which are useful as described herein. A number of immunogens are used to produce antibodies that specifically bind differentially expressed polypeptides. Full-length differentially expressed polypeptides are suitable immunogens. Typically, the immunogen of interest is a peptide of at least about 3 amino acids, more typically the peptide is at least 5 amino acids in length, the fragment is at least 10 amino acids in length and typically the fragment is at least 15 amino acids in length. The peptides can be coupled to a carrier protein (e.g., as a fusion protein), or are recombinantly expressed in an immunization vector. Antigenic determinants on peptides to which antibodies bind are typically 3 to 10 amino acids in length. Naturally occurring polypeptides are also used either in pure or impure form. Recombinant polypeptides are expressed in eukaryotic or prokaryotic cells and purified using standard techniques. The polypeptide, or a synthetic version thereof, is then injected into an animal capable of producing antibodies. Either monoclonal or polyclonal antibodies can be generated for subsequent use in immunoassays to measure the presence and quantity of the polypeptide.

These antibodies find use in a number of applications. For example, the differentially expressed antibodies can be coupled to standard affinity chromatography columns and used to purify differentially expressed proteins as further described below. The antibodies can also be used as blocking polypeptides, as outlined above, since they will specifically bind to the differentially expressed protein.

The anti-differentially expressed protein antibodies can comprise polyclonal antibodies. Methods for producing polyclonal antibodies are known to those of skill in the art. In brief, an immunogen, for example, a purified polypeptide, a polypeptide coupled to an appropriate carrier (e.g., GST and keyhole limpet hemocyanin), or a polypeptide incorporated into an immunization vector such as a recombinant vaccinia virus (see, U.S. Pat. No. 4,722,848) is mixed with an adjuvant and animals are immunized with the mixture. The animal's immune response to the immunogen preparation is monitored by taking test bleeds and determining the titer of reactivity to the polypeptide of interest. When appropriately high titers of antibody to the immunogen are obtained, blood is collected from the animal and antisera are prepared. Further fractionation of the antisera to enrich for antibodies reactive to the polypeptide is performed where desired. See, e.g., Coligan, 1991, Current Protocols in Immunology Wiley/Greene, NY; and Harlow and Lane, 1989, Antibodies: A Laboratory Manual Cold Spring Harbor Press, NY., each incorporated herein by reference

Antibodies, including binding fragments and single chain recombinant versions thereof, against predetermined fragments of differentially expressed proteins are raised by immunizing animals, e.g., with conjugates of the fragments with carrier proteins as described above.

The anti-differentially expressed protein antibodies can, alternatively, be monoclonal antibodies. The monoclonal antibodies are prepared from cells secreting the desired antibody. These antibodies are screened for binding to normal or modified polypeptides, or screened for agonistic or antagonistic activity, e.g., activity mediated through the differentially expressed proteins. In some instances, it is desirable to prepare monoclonal antibodies from various mammalian hosts, such as mice, rodents, primates, and humans. Description of techniques for preparing such monoclonal antibodies are found in, e.g., Stites et al. (eds.) Basic and Clinical Immunology (4th ed.) Lange Medical Publications, Los Altos, Calif., and references cited therein; Harlow and Lane, Supra; Goding, 1986, Monoclonal Antibodies: Principles and Practice (2d ed.) Academic Press, New York, N.Y.; and Kohler and Milstein, Nature 256: 495-497, 1975, each incorporated herein by reference.

The immunizing agent will typically include the differentially expressed protein polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies. Principles and Practice, Academic Press, 1986, pp. 59-103, incorporated herein by reference). Immortalized cell lines are usually transformed mammalian cells, particularly mycloma cells of rodent, bovine and human origin. Usually, rat or mouse myeloma cell lines are employed. The hybridoma cells can be cultured in a suitable culture medium that contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Immortalized cell lines are those that fuse efficiently, support stable high level expression of antibody by the selected antibody-producing cells, and are sensitive to a medium such as HAT medium. More immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133: 3001, 1984; Brodeur et al., Monoclonal Antibody Production Techniques and Applications, Marcel Dekker, Inc., New York, 1987, pp. 51-63, each incorporated herein by reference).

The culture medium in which the hybridoma cells are cultured can then be assayed for the presence of monoclonal antibodies directed against differentially expressed protein. The binding specificity of monoclonal antibodies produced by the hybridoma cells is determined by immunoprecipitation or by an in vitro binding assay, such as radioimmunoassay (RIA) or enzyme-linked immunosorbent assay (ELISA). Such techniques and assays are known in the art. The binding affinity of the monoclonal antibody can, for example, be determined by the Scatchard analysis of Munson and Pollard, Anal. Biochem. 107: 220, 1980, incorporated herein by reference.

After the desired hybridoma cells are identified, the clones can be subcloned by limiting dilution procedures and grown by standard methods (Goding, supra). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Other suitable techniques involve selection of libraries of recombinant antibodies in phage or similar vectors. See, Huse et al., Science 246: 1275-1281, 1989; and Ward, et al., Nature 341: 544-546, 1989, each incorporated herein by reference.

Also, recombinant immunoglobulins can be produced. See, U.S. Pat. No. 4,816,567; and Queen et al., Proc. Nat'l Acad. Sci. USA 86: 10029-10033, 1989, each incorporated herein by reference.

Briefly, nucleic acids encoding light and heavy chain variable regions, optionally linked to constant regions, are inserted into expression vectors. The light and heavy chains can be cloned in the same or different expression vectors. The DNA segments encoding antibody chains are operably linked to control sequences in the expression vector(s) that ensure the expression of antibody chains. Such control sequences include a signal sequence, a promoter, an enhancer, and a transcription termination sequence. Expression vectors are typically replicable in the host organisms either as episomes or as an integral part of the host chromosome.

E. coli is one procaryotic host useful for expressing antibodies. Other microbial hosts suitable for use include bacilli, such as Bacillus subtilus, and other enterobacteriaceae, such as Salmonella, Serratia, and various Pseudomonas species. In these prokaryotic hosts, one can also make expression vectors, which typically contain expression control sequences compatible with the host cell (e.g., an origin of replication) and regulatory sequences such as a lactose promoter system, a tryptophan (trp) promoter system, a beta-lactamase promoter system, or a promoter system from phage lambda.

Other microbes, such as yeast, can also be used for expression. Saccharomyces is one host, with suitable vectors having expression control sequences, such as promoters, including 3-phosphoglycerate kinase or other glycolytic enzymes, and an origin of replication, termination sequences and the like as desired.

Mammalian tissue cell culture can also be used to express and produce the antibodies (See Winnacker, From Genes to Clones, VCH Publishers, N.Y., 1987, incorporated herein by reference). Eukaryotic cells are useful because a number of suitable host cell lines capable of secreting intact antibodies have been developed. Suitable host cells for expressing nucleic acids encoding the immunoglobulins include: monkey kidney CV1 line transformed by SV40 (COS-7, ATCC CRL 1651); human embryonic kidney line (293) (Graham et al., J. Gen. Virol. 36: 59, 1977); baby hamster kidney cells (BHK, ATCC CCL 10); Chinese hamster ovary-cells-DHFR (CHO, Urlaub and Chasin, Proc. Natl. Acad. Sci. U.S.A. 77: 4216, 1980); mouse sertoli cells (TM4, Mather, Biol. Reprod. 23: 243-251, 1980); monkey kidney cells (CV1 ATCC CCL 70); african green monkey kidney cells (VERO-76, ATCC CRL 1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); and, TR1 cells (Mather et al., Annals N.Y. Acad. Sci. 383: 44-46, 1982); baculovirus cells. Each citation incorporated herein by reference

The vectors containing the polynucleotide sequences of interest (e.g., the heavy and light chain encoding sequences and expression control sequences) can be transferred into the host cell. Calcium chloride transfection is commonly utilized for prokaryotic cells, whereas calcium phosphate treatment or electroporation can be used for other cellular hosts. See generally Sambrook et al., Molecular Cloning: A Laboratory Manual Cold Spring Harbor Press, 2d ed., 1989, incorporated herein by reference. When heavy and light chains are cloned on separate expression vectors, the vectors are co-transfected to obtain expression and assembly of intact immunoglobulins. After introduction of recombinant DNA, cell lines expressing immunoglobulin products are cell selected. Cell lines capable of stable expression are useful (i.e., undiminished levels of expression after fifty passages of the cell line).

Once expressed, the whole antibodies, their dimers, individual light and heavy chains, or other immunoglobulin forms can be purified according to standard procedures of the art, including ammonium sulfate precipitation, affinity columns, column chromatography, gel electrophoresis and the like. See generally Scopes, Protein Purification, Springer-Verlag, N.Y., 1982, incorporated herein by reference. Substantially pure immunoglobulins are of at least about 90 to 95% homogeneity, and are typically 98 to 99% homogeneity or more.

Frequently, the polypeptides and antibodies will be labeled by joining, either covalently or non-covalently, a substance which provides for a detectable signal. A wide variety of labels and conjugation techniques are known and are reported extensively in both the scientific and patent literature. Thus, an antibody used for detecting an analyte can be directly labeled with a detectable moiety, or can be indirectly labeled by, for example, binding to the antibody a secondary antibody that is, itself directly or indirectly labeled.

Antibodies are also used for affinity chromatography in isolating differentially expressed proteins. Columns are prepared, e.g., with the antibodies linked to a solid support, e.g., particles, such as agarose, Sephadex, or the like, where a cell lysate is passed through the column, washed, and treated with increasing concentrations of a mild denaturant, whereby purified differentially expressed polypeptides are released.

A further approach for isolating DNA sequences which encode a human monoclonal antibody or a binding fragment thereof is by screening a DNA library from human B cells according to the general protocol outlined by Huse et al., Science 246:1275-1281, 1989, incorporated herein by reference, and then cloning and amplifying the sequences which encode the antibody (or binding fragment) of the desired specificity. Such B cells can be obtained from a human immunized with the desired antigen, fragments, longer polypeptides containing the antigen or fragments or anti-idiotypic antibodies. Optionally, such B cells are obtained from an individual who has not been exposed to the antigen. B cell can also be obtained from transgenic non-human animals expressing human immunoglobulin sequences. The transgenic non-human animals can be immunized with an antigen or collection of antigens. The animals can also be unimmunized. B cell mRNA sequences encoding human antibodies are used to generate cDNA using reverse transcriptase. The V region encoding segments of the cDNA sequences are then cloned into a DNA vector that directs expression of the antibody V regions. Typically the V region sequences are specifically amplified by PCR prior to cloning. Also typically, the V region sequences are cloned into a site within the DNA vector that is constructed so that the V region is expressed as a fusion protein. Examples of such fusion proteins include m13 coliphage gene 3 and gene 8 fusion proteins. The collection of cloned V region sequences is then used to generate an expression library of antibody V regions. To generate an expression library, the DNA vector comprising the cloned V region sequences is used to transform eukaryotic or prokaryotic host cells. In addition to V regions, the vector can optionally encode all or part of a viral genome, and can comprise viral packaging sequences. In some cases the vector does not comprise an entire virus genome, and the vector is then used together with a helper virus or helper virus DNA sequences. The expressed antibody V regions are found in, or on the surface of, transformed cells or virus particles from the transformed cells. This expression library, comprising the cells or virus particles, is then used to identify V region sequences that encode antibodies, or antibody fragments reactive with predetermined antigens. To identify these V region sequences, the expression library is screened or selected for reactivity of the expressed V regions with the predetermined antigens. The cells or virus particles comprising the cloned V region sequences, and having the expressed V regions, are screened or selected by a method that identifies or enriches for cells or virus particles that have V regions reactive (e.g., binding association or catalytic activity) with a predetermined antigen. For example, radioactive or fluorescent labeled antigen that then binds to expressed V regions can be detected and used to identify or sort cells or virus particles. Antigen bound to a solid matrix or bead can also be used to select cells or virus particles having reactive V regions on the surface. The V region sequences thus identified from the expression library can then be used to direct expression, in a transformed host cell, of an antibody or fragment thereof, having reactivity with the predetermined antigen.

The protocol described by Huse is rendered more efficient in combination with phage-display technology. See, e.g., Dower et al., WO 91/17271 and McCafferty et al., WO 92/01047, U.S. Pat. Nos. 5,871,907, 5,858,657, 5,837,242, 5,733,743 and 5,565,332, each incorporated herein by reference. In these methods, libraries of phage are produced in which members (display packages) display different antibodies on their outer surfaces. Antibodies are usually displayed as Fv or Fab fragments. Phage displaying antibodies with a desired specificity can be selected by affinity enrichment to the antigen or fragment thereof. Phage display combined with immunized transgenic non-human animals expressing human immunoglobulin genes can be used to obtain antigen specific antibodies even when the immune response to the antigen is weak.

In a variation of the phage-display method, human antibodies having the binding specificity of a selected murine antibody can be produced. See, for example, WO 92/20791, incorporated herein by reference. In this method, either the heavy or light chain variable region of the selected murine antibody is used as a starting material. If, for example, a light chain variable region is selected as the starting material, a phage library is constructed in which members display the same light chain variable region (i.e., the murine starting material) and a different heavy chain variable region. The heavy chain variable regions are obtained from a library of rearranged human heavy chain variable regions. A phage showing strong specific binding (e.g., at least 10⁸ and typically at least 10⁹ M⁻¹) can then be selected. The human heavy chain variable region from this phage then serves as a starting material for constructing a further phage library. In this library, each phage displays the same heavy chain variable region (i.e., the region identified from the first display library) and a different light chain variable region. The light chain variable regions are obtained from a library of rearranged human variable light chain regions. Again, phage showing strong specific binding for the selected are selected. Artificial antibodies that are similar to human antibodies can be obtained from phage display libraries that incorporate random or synthetic sequences, for example, in CDR regions.

In another embodiment, fragments of antibodies against differentially expressed protein or protein analogs are provided. Typically, these fragments exhibit specific binding to the differentially expressed protein receptor similar to that of a complete immunoglobulin. Antibody fragments include separate heavy chains, light chains F_{ab}, F_{ab′} F_{(ab′)2} and Fᵥ. Fragments are produced by recombinant DNA techniques, or by enzymic or chemical separation of intact immunoglobulins.

The antibodies can be monovalent antibodies. Methods for preparing monovalent antibodies are well known in the art. For example, one method involves recombinant expression of immunoglobulin light chain and modified heavy chain. The heavy chain is truncated generally at any point in the F_{c} region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

An alternative approach is the generation of humanized immunoglobulins by linking the CDR regions of non-human antibodies to human constant regions by recombinant DNA techniques. See U.S. Pat. No. 5,585,089, incorporated herein by reference. Humanized forms of non-human (e.g., murine) antibodies are immunoglobulins, immunoglobulin chains or fragments thereof (such as Fᵥ, F_{ab}, F_{ab′}, F_{ab2} or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fᵥ framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an F_{c} region, typically that of a human immunoglobulin. See Jones et al., Nature, 321: 522-525, 1986; Riechmann et al., Nature, 332: 323-329, 1988; and Presta, Curr. Op. Struct. Biol., 2: 593-596, 1992, each incorporated herein by reference.

Chimeric and humanized antibodies have the same or similar binding specificity and affinity as a mouse or other nonhuman antibody that provides the starting material for construction of a chimeric or humanized antibody. Chimeric antibodies are antibodies whose light and heavy chain genes have been constructed, typically by genetic engineering, from immunoglobulin gene segments belonging to different species. For example, the variable (V) segments of the genes from a mouse monoclonal antibody can be joined to human constant (C) segments, such as IgG₁ and IgG₄. Human isotype IgG₁ is typically used. A typical chimeric antibody is thus a hybrid protein consisting of the V or antigen-binding domain from a mouse antibody and the C or effector domain from a human antibody.

Humanized antibodies have variable region framework residues substantially from a human antibody (termed an acceptor antibody) and complementarity determining regions substantially from a mouse-antibody (referred to as the donor immunoglobulin). See, Queen et al., Proc. Natl. Acad. Sci. U.S.A. 86: 10029-10033, 1989 and WO 90/07861, U.S. Pat. No. 5,693,762, U.S. Pat. No. 5,693,761, U.S. Pat. No. 5,585,089, U.S. Pat. No. 5,530,101 and U.S. Pat. No. 5,225,539, each incorporated herein by reference. The constant region(s), if present, are also substantially or entirely from a human immunoglobulin. The human variable domains are usually chosen from human antibodies whose framework sequences exhibit a high degree of sequence identity with the murine variable region domains from which the CDRs were derived. The heavy and light chain variable region framework residues can be derived from the same or different human antibody sequences. The human antibody sequences can be the sequences of naturally occurring human antibodies or can be consensus sequences of several human antibodies. See WO 92/22653, incorporated herein by reference. Certain amino acids from the human variable region framework residues are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid should usually be substituted by the equivalent framework amino acid from the mouse antibody when it is reasonably expected that the amino acid: (1) noncovalently binds antigen directly, (2) is adjacent to a CDR region, (3) otherwise interacts with a CDR region (e.g. is within about 6 A of a CDR region), or (4) participates in the VL-VH interface.

Other candidates for substitution are acceptor human framework amino acids that are unusual for a human immunoglobulin at that position. These amino acids can be substituted with amino acids from the equivalent position of the mouse donor antibody or from the equivalent positions of more typical human immunoglobulins. Other candidates for substitution are acceptor human framework amino acids that are unusual for a human immunoglobulin at that position. The variable region frameworks of humanized immunoglobulins usually show at least 85% sequence identity to a human variable region framework sequence or consensus of such sequences.

Human antibodies can also be produced using various techniques known in the art, including phage display libraries discussed above (Hoogenboom and Winter, J. Mol. Biol., 227: 381, 1991; Marks et al., J. Mol. Biol., 222: 581, 1991). The techniques of Cole et al. and Boerner et al. are also available for the preparation of human monoclonal antibodies (Cole et al., Monoclonal Antibodies and Cancer Therapy, Alan R. Liss, p. 77, 1985 and Boerner et al., J. Immunol., 147: 86-95, 1991). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016; see also Marks et al., Bio/Technology 10: 779-783, 1992; Lonberg et al., Nature, 368: 856-859, 1994; Morrison, Nature, 368: 812-13, 1994; Fishwild et al., Nature Biotechnology, 14: 845-51, 1996; Neuberger, Nature Biotechnology, 14: 826, 1996; Lonberg and Huszar, Intern. Rev. Immunol., 13: 65-93, 1995. Each citation incorporated herein by reference.

Bispecific antibodies are monoclonal, typically human or humanized, antibodies that have binding specificities for at least two different antigens. In the present case, one of the binding specificities is for the differentially expressed protein, the other one is for any other antigen, and for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities Milstein and Cuello, Nature, 305: 537-539, 1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J, 10: 3655-3659, 1991. Each citation incorporated herein by reference.

Antibody variable domains with the desired binding specificities (antibody-antigen combining sites) can be fused to immunoglobulin constant domain sequences. The fusion is typically with an immunoglobulin heavy-chain constant domain, comprising at least part of the hinge, CH2, and CH3 regions. Typically the first heavy-chain constant region (CH1) contains the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121: 210, 1986, incorporated herein by reference.

Heteroconjugate antibodies are also within the scope of the present methods and compositions. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980. Each citation incorporated herein by reference.

The anti-differentially expressed protein antibodies have various utilities. For example, anti-differentially expressed protein antibodies can be used in diagnostic assays for a differentially expressed protein, e.g., detecting its expression in specific cells, tissues, or serum. Various diagnostic assay techniques can be used, such as competitive binding assays, direct or indirect sandwich assays and immunoprecipitation assays conducted in either heterogeneous or homogeneous phases (Zola, Monoclonal Antibodies: A Manual of Techniques, CRC Press, Inc., pp. 147-158, 1987). The antibodies used in the diagnostic assays can be labeled with a detectable moiety. The detectable moiety should be capable of producing, either directly or indirectly, a detectable signal. For example, the detectable moiety can be a radioisotope, such as 3H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety can be employed, including those methods described by Hunter et al., Nature, 144: 945, 1962; David et al., Biochemistry, 13: 1014, 1974; Pain et al., J. Immunol. Meth., 40: 219, 1981; and Nygren, J. Histochem. and Cytochem., 30: 407, 1982. Each citation incorporated herein by reference.

Anti-differentially expressed protein antibodies also are useful for the affinity purification of differentially expressed protein from recombinant cell culture or natural sources. In this process, the antibodies against differentially expressed protein are immobilized on a suitable support, such a Sephadex resin or filter paper, using methods well known in the art. The immobilized antibody then is contacted with a sample containing the differentially expressed protein to be purified, and thereafter the support is washed with a suitable solvent that will remove substantially all the material in the sample except the differentially expressed protein, which is bound to the immobilized antibody. Finally, the support is washed with another suitable solvent that will release the differentially expressed protein from the antibody.

Pharmaceutical Compositions and Methods of Administration

The anti-differentially expressed protein antibodies can also be used in treatment. In some methods, the genes encoding the antibodies are provided, such that the antibodies bind to and modulate the differentially expressed protein within the cell. In other methods, a therapeutically effective amount of a differentially expressed protein, agonist or antagonist is administered to a patient. A “therapeutically effective amount”, “pharmacologically acceptable dose”, “pharmacologically acceptable amount” means that a sufficient amount of an immunosuppressive agent or combination of agents is present to achieve a desired result, e.g., preventing, delaying, inhibiting or reversing a symptom of a disease or disorder or the progression of disease or disorder when administered in an appropriate regime.

Pharmaceutically acceptable carriers are determined in part by the particular composition being administered, as well as by the particular method used to administer the composition. Accordingly, there is a wide variety of suitable formulations of pharmaceutical compositions (see, e.g., Remington 's Pharmaceutical Sciences, 18^{th} ed., 1990, incorporated herein by reference). The pharmaceutical compositions generally comprise a differentially expressed protein, agonist or antagonist in a form suitable for administration to a patient. The pharmaceutical compositions are generally formulated as sterile, substantially isotonic and in full compliance with all Good Manufacturing Practice (GMP) regulations of the U.S. Food and Drug Administration.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art.

In some methods, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like, particularly the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The nucleic acids, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the packaged nucleic acid with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Compositions can be administered, for example, by intravenous infusion, orally, topically, intraperitoneally, intravesically or intrathecally. Formulations for injection can be presented in unit dosage form, e.g., in ampules or in multidose containers, with an added preservative.

Injection solutions and suspensions can be prepared from sterile powders, granules, and tablets of the kind previously described. Cells transduced by the packaged nucleic acid as described above in the context of ex vivo therapy can also be administered intravenously or parenterally as described above.

The dose administered to a patient should be sufficient to effect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the vector to be administered in the treatment or prophylaxis of conditions resulting from expression of the differentially expressed proteins of the methods and compositions, the physician evaluates circulating plasma levels of the vector, vector toxicities, progression of the disease, and the production of anti-vector antibodies. In general, the dose equivalent of a naked nucleic acid from a vector is from about 1 μg to 100 μg for a typical 70 kilogram patient, and doses of vectors which include a retroviral particle are calculated to yield an equivalent amount of therapeutic nucleic acid.

For administration, inhibitors and transduced cells can be administered at a rate determined by the LD₅₀ of the inhibitor, vector, or transduced cell type, and the side-effects of the inhibitor, vector or cell type at various concentrations, as applied to the mass and overall health of the patient. Administration can be accomplished via single or divided doses.

Transduced cells are prepared for reinfusion according to established methods. See Abrahamsen et al., J. Clin. Apheresis 6: 48-53, 1991; Carter et al., J. Clin. Arpheresis 4: 113-117, 1998; Aebersold et al., J. Immunol. Meth. 112: 1-7, 1998; Muul et al., J. Immunol. Methods, 101: 171-181, 1987; and Carter et al., Transfusion 27: 362-365, 1987, each incorporated herein by reference. After a period of about 2-4 weeks in culture, the cells should number between 1×10⁸ and 1×10¹². In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent.

Kits

The differentially expressed protein, agonist or antagonist or their homologs are useful tools for examining expression and regulation of signaling in endothelial cells via the PAR1 pathway. Reagents that specifically hybridize to nucleic acids encoding differentially expressed proteins (including probes and primers of the differentially expressed proteins), and reagents that specifically bind to the differentially expressed proteins, e.g., antibodies, are used to examine expression and regulation.

Nucleic acid assays for the presence of differentially expressed proteins in a sample include numerous techniques are known to those skilled in the art, such as Southern analysis, northern analysis, dot blots, RNase protection, S1 analysis, amplification techniques such as PCR and LCR, high density oligonucleotide array analysis, and in situ hybridization. In in situ hybridization, for example, the target nucleic acid is liberated from its cellular surroundings in such as to be available for hybridization within the cell while preserving the cellular morphology for subsequent interpretation and analysis. The following articles provide an overview of the art of in situ hybridization: Singer et al., Biotechniques 4: 230-250, 1986; Haase et al., Methods in Virology, vol. VII, pp. 189-226, 1984; and Nucleic Acid Hybridization: A Practical Approach (Hames et al., eds. 1987), each incorporated herein by reference. In addition, a differentially expressed protein can be detected with the various immunoassay techniques described above. The test sample is typically compared to both a positive control (e.g., a sample expressing recombinant differentially expressed protein) and a negative control.

Kits for screening endothelial cell activity modulators. Such kits can be prepared from readily available materials and reagents are provided. For example, such kits can comprise any one or more of the following materials: the differentially expressed proteins, agonists, or antagonists, reaction tubes, and instructions for testing the activities of differentially expressed genes. A wide variety of kits and components can be prepared depending upon the intended user of the kit and the particular needs of the user. For example, the kit can be tailored for in vitro or in vivo assays for measuring the activity of a differentially expressed proteins or endothelial cell activity modulators.

Kits comprising probe arrays as described above are provided. Optional additional components of the kit include, for example, other restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions.

Usually, the kits also contain instructions for carrying out the methods.

Other embodiments and uses will be apparent to one skilled in the art in light of the present disclosures.

The following are provided by way of illustration, not by limitation.

### EXAMPLES

### Example 1

Activation of Endothelial Cell Protease Activated Receptor 1 by the Protein C Pathway

The coagulant and inflammatory exacerbation in sepsis is counterbalanced by the protective protein C (PC) pathway. Here we show that activated PC (APC) utilizes endothelial cell PC receptor (EPCR) as a coreceptor for cleavage of protease activated receptor (PAR)1 on endothelial cells. Gene profiling demonstrates that PAR1 signaling accounts for all APC induced protective genes, including the immuno-modulatory monocyte chemoattractant protein-1 that is selectively induced by PAR1, but not by PAR2 activation. The unexpected finding that the prototypical thrombin receptor is the target for APC signaling provides novel insight into APC's protective effects in sepsis.

PAR1 deficient murine fibroblasts are not activated by proteases, unless transfected with an appropriate PAR (Camerer, et al., Proc Natl Acad Sci USA, 97: 5255, 2000; Riewald, W. Ruf, Proc Natl Acad Sci USA, 98: 7742, 2001). We exploited the unresponsiveness of these cells to APC to characterize the requirement for protease signaling by APC. Stimulation with APC was performed in the presence of 100 nM hirudin, previously shown to block all cell surface thrombin-mediated PAR1 signaling (M. Riewald et al., Blood, 97: 3109, 2001). PAR1-deficient fibroblasts were responsive to 20 nM APC only when EPCR was co-expressed with a PAR (FIG. 1A). Expression of EPCR or PAR2 alone or co-expression of PAR2 with an EPCR mutant deficient in APC binding (P. C. Liaw, et al., J Biol Chem, 276: 8364, 2001) failed to support APC signaling (FIG. 1A), demonstrating that only EPCR bound APC can efficiently activate PARs.

Co-expression of EPCR and PAR1 also resulted in responsiveness to APC (FIG. 1B). Like thrombin signaling, APC signaling was inhibited by cleavage blocking antibodies to PAR1, whereas anti-PAR1 antibodies did not prevent signaling by direct PAR1 or other G-protein coupled receptor agonists, excluding non-specific PAR1 desensitization. Thus, APC signals through a proteolytic mechanism and not through a protease independent receptor crosstalk between EPCR and PARs. There was no difference in the APC dose response of PAR1 versus PAR2 activation, indicating that the presentation of APC by binding to EPCR, rather than specific features in the scissile bond of the PARs, determine efficiency of PAR cleavage. The finding that the prototypical thrombin receptor PAR1 can be activated by other proteases is in line with recent data indicating that coagulation factor Xa also cleaves PAR1 (Camerer, et al., Proc Natl Acad Sci USA, 97: 5255, 2000; Riewald, W. Ruf, Proc Natl Acad Sci USA, 98: 7742, 2001; M. Riewald et al., Blood, 97: 3109, 2001). EPCR has a potential cytoplasmic palmitoylation site that can influence the co-signaling properties of the receptor. However, mutation of the single cytoplasmic Cys to Ser did not reduce signaling of EPCR-bound APC through PAR1 or PAR2 (FIG. 1A). Thus, palmitoylation of EPCR is not a strict requirement for APC-dependent cleavage of PARs.

Although the heterologous over-expression experiments clearly demonstrated that EPCR-bound APC could activate PAR1 and -2, it remained to be established whether EPCR is properly positioned on the surface of primary endothelial cells to support the cleavage of PARs by APC. Stimulation of endothelial cells with APC induced MAP kinase Erk1/2 phosphorylation, a common response of PAR1 and PAR2 signaling (FIGS. 1B and 1C). Active site blocked APC failed to induce MAP kinase phosphorylation, demonstrating that proteolytic activity of APC is necessary for PAR cleavage on primary cells. Moreover, Erk1/2 phosphorylation in response to APC, but not thrombin, was inhibited by a 10-fold molar excess of active site-blocked APC that competes for EPCR binding, confirming receptor dependence of APC signaling in endothelial cells. Cleavage blocking anti-PAR1 antibodies inhibited Erk1/2 phosphorylation in response to APC, without affecting the response to the PAR2 agonist peptide. Thus, MAP kinase phosphorylation by the PC pathway on endothelial cells is dependent on PAR1.

To determine whether PAR activation accounts for APC-dependent gene induction in endothelial cells, large scale mRNA expression profiles were determined for endothelial cells stimulated with APC or with selective agonist peptides for PAR1 or PAR2 for 90 minutes. This time was chosen to capture both early transcription related and delayed effector gene induction events. Based on three independent experiments, 1% of the ˜7000 represented genes were reproducibly upregulated by stimulation with APC or a PAR agonist (FIG. 2). PAR1 and PAR2 agonists induced most of these genes to a similar degree (FIG. 2A). The most prominent exception was the transcript of monocyte chemoattractant protein-1 (MCP-1), which was only induced by the PAR1 agonist.

Overall, gene induction by APC correlated with direct agonist stimulation of PAR1 (FIG. 2B) as well as PAR2 (FIG. 2C). Some genes were induced by PAR agonist peptides, but not by APC stimulation. Most significantly, none of the transcripts was induced by APC, but not by the PAR1 agonist, and APC stimulation resulted in the potent upregulation of the MCP-1 gene (FIG. 2, B and C). An additional 1% of the genes represented on the microarray showed more inter-experimental variability in agonist induction and did not meet the stringent criteria for inclusion in FIG. 2, A-C. Analysis of these additional genes further showed that none of the PAR2 selective transcripts was induced by APC, but APC upregulated other PAR1 selective genes (Table 1). Hierarchical clustering of the larger gene set-confirmed the similarity between APC signaling and the PAR1 response. Gene profiling thus demonstrates that all transcriptional responses to APC signaling of endothelial cells are accounted for by PAR1 signaling.

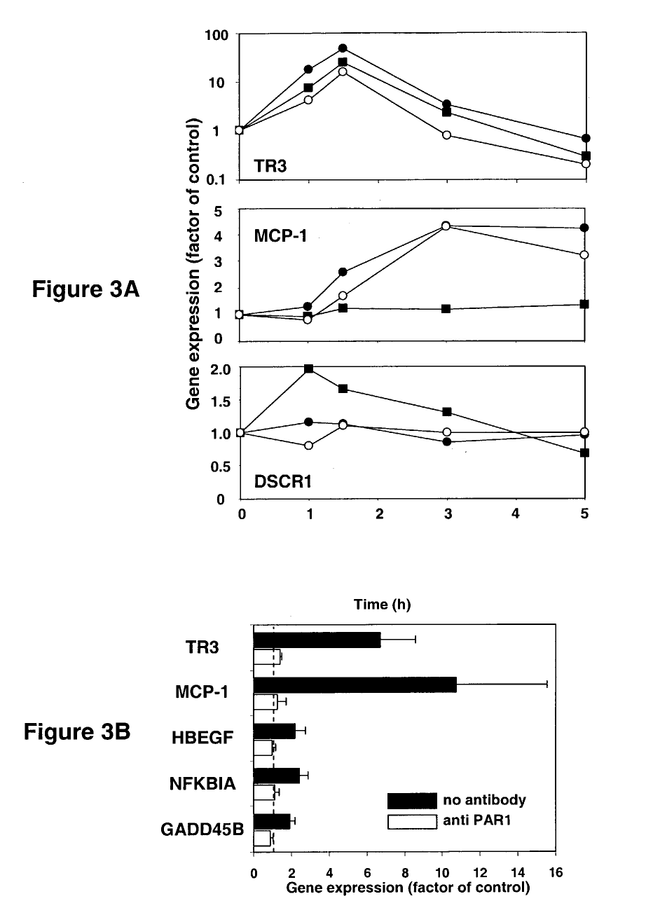

Time course experiments in primary endothelial cells confirmed that MCP-1 was similarly induced by APC and the PAR1 agonist, but not by direct activation of PAR2, whereas all three agonists induced the nuclear receptor TR3 with similar kinetics (FIG. 3A). The transcript for DSCR1, a negative regulator of calcineurin was selected as an example for a gene that was not induced by APC stimulation. Time course experiments confirmed the gene chip data and further demonstrate that this transcript shows selective upregulation by PAR2 stimulation (FIG. 3A). Thus, APC cannot induce a response that is typical for PAR2 agonist stimulation. As expected, anti-PAR1 antibodies blocked the induction of the PAR1 specific MCP-1 gene by APC. Anti-PAR1 antibodies also inhibited the APC-dependent induction of genes responsive to either PAR1 or PAR2 stimulation, such as TR3, HBEGF, NFKBIa, and GADD45B (FIG. 3B). Thus, PAR1 selective and PAR2 permissive APC responses were blocked by anti-PAR1 antibodies, demonstrating that PAR2 cannot substitute for PAR1 in APC signaling of endothelial cells. Simultaneous expression of PAR1 and PAR2 in knock-out fibroblasts did not produce a similar PAR1 specificity of APC signaling, excluding that PAR1 coexpression restricts PAR2 cleavage by EPCR-bound APC. The selective APC mediated activation of PAR1 in endothelial cells can result from a cell-type specific colocalization of PAR1 and EPCR in a particular microenvironment or from potential post-translational modifications of PAR2 (S. J. Compton, et al., Br J Pharmacol, 134: 705, 2001) that can restrict APC cleavage.

Pretreatment of cells with high concentrations of APC had been described to inhibit inflammatory signaling and induce cell survival pathways in endothelial cells and monocytes (D. E. Joyce, et al., J Biol Chem, 276: 11199, 2001; Shu et al., FEBS Lett., 477: 208, 2000; S. T. Grey et al., J Immunol 153: 3664, 1994). APC signaling and direct PAR activation similarly induced an early transcriptional response that can be interpreted as counter-regulatory mechanisms of proinflammatory signaling pathways (Table 2). These include negative regulators of the G-protein coupled receptor (AKAP12, gravin), the MAP kinase/egr (DUSP1 and 5, NAB 1), and the NFκB (NFκBIα) pathways. The NFκB-dependent genes TNFAIP3 (A20) (E. G. Lee et al., Science 289: 2350, 2000) and ZFP36 (TTP) (E. Carballo, et al., Science, 281: 1001, 1998) are crucial to terminate TNF signaling and targeted deletions of these genes result in chronic inflammation in vivo. NFκB-dependent genes with known anti-apoptotic functions were also similarly upregulated by APC and PAR signaling. TNFAIP3 (A20), IER3, and GADD45B are known as TNF-induced antiapoptotic genes. Two additional PAR and APC induced anti-apoptotic genes, i.e. A1 and IAP-1, were also upregulated after long term (16 hours) stimulation with 10-fold higher concentrations of APC (D. E. Joyce, et al., J. Biol. Chem., 276: 11199, 2001), but other APC regulated genes in the latter study were not changed at the early time point of our experiments. The concordant upregulation of protective genes by PAR1 agonists and APC provides strong evidence that all anti-inflammatory and anti-apoptotic effects of APC signaling are PAR mediated in endothelial cells.

Activation of the prototypical thrombin receptor PAR1 by APC can be relevant even if the generation of APC is thrombin dependent. Infusion of low concentrations of thrombin into primates produces a prominent activation of the protein C pathway without major signs of platelet activation, a highly sensitive thrombin-mediated PAR1 response in vivo (S. R. Hanson et al., J Clin Invest, 92: 2003, 1993). The absence of detectable platelet PAR1 activation by thrombin can result from the presence of high concentrations of inhibitors and competing substrates, foremost fibrinogen, that occupy the PAR1 interactive exosite I of thrombin (T. K. Vu, et al., Nature 353: 674, 1991). Endothelial cell thrombomodulin also occupies exosite I (P. Fuentes-Prior et al., Nature 404: 518, 2000) and thus inhibits PAR1 activation by thrombin on cell surfaces. Rather, thrombin activates the protein C pathway (S. R. Hanson et al., J Clin Invest, 92: 2003, 1993) due to thrombin's recruitment to endothelial cell thrombomodulin that inhibits thrombin-dependent PAR signaling (B. W. Grinnell, D. T. Berg, Am J Physiol, 270: H603, 1996). Given that thrombin recruited to thrombomodulin activates EPCR-bound PC(C. T. Esmon et al., Thromb Haemost, 82: 251, 1999) and that EPCR-bound APC activates PAR1, the physiological activation of the PC pathway emerges as a highly relevant activator of protective PAR1 signaling on endothelial cells.

The coreceptor function of EPCR in protective APC signaling is well supported by the anti-inflammatory functions of EPCR in sepsis models (F. B. Taylor et al., Blood, 95: 1680, 2000). In severe sepsis endothelial dysfunction and the downregulation of thrombomodulin promote a widespread reduction in PC activation and increased thrombin production, but EPCR remains detectable on thrombomodulin-depleted endothelial cells (S. N. Faust et al., N Engl J Med, 345: 408, 2001). The finding that even in the presence of normal PC plasma levels therapeutically administered APC is beneficial in severe sepsis (G. R. Bernard et al., N Engl J Med, 344: 699, 2001) can thus be explained by the utilization of residual EPCR for protective APC signaling. Whereas this exogenous APC targets EPCR expressing endothelial cells throughout the vasculature, thrombin signaling is locally more restricted to sites of ongoing coagulation activation. Furthermore, thrombin activates other cells, leads to platelet secretion and aggregation, and generates fibrin, supporting the recruitment and activation of inflammatory cells. In sum, thrombin's effects are thus unlikely to reproduce the endothelium-restricted, protective PAR1 signaling of APC in the escalation of the sepsis syndrome.

An unexpected finding was the PAR1 selective induction of MCP-1 in endothelial cells. In addition to direct protective effects of APC on the endothelial cells, the induction of MCP-1 by APC can indicate indirect anti-inflammatory effects for APC signaling in vivo. MCP-1 has a role in monocyte recruitment to local sites of inflammation that is coupled to potent anti-inflammatory regulation of the monocyte cytokine response (S. A. Luther, et al., Nat Immunol, 2: 102, 2001). The importance of local cytokine networks for sepsis outcome is well appreciated and MCP-1 can locally control the inflammatory response of the innate immune system. In addition, MCP-1 influences the acquired immune system by inducing TH, polarization and the associated upregulation of anti-inflammatory cytokines, including interleukin 10 (C. Gerard, et al., Nat Immunol, 2: 108, 2001). In both systemically and locally induced sepsis models, MCP-1 administration is protective and neutralization of MCP-1 increases lethality (D. A. Zisman et al., JClin Invest, 99: 2832, 1997; C. L. Bone-Larson et al., Am JPathol, 157: 1177, 2000), while interleukin 10 counteracts the systemic inflammatory response (M. E. Sewnath et al., Jlmmunol, 166: 6323, 2001). The presented data thus link the PC pathway specific activation of PAR1 to a local and systemic immuno-modulatory chemokine response of relevance for controlling the host defense in sepsis.

The inflammatory cytokine production in sepsis disables the physiological anticoagulant pathway by downregulating thrombomodulin, but EPCR remains detectable on thrombomodulin-depleted endothelial cells (S. N. Faust et al., N Engl J Med, 345: 408, 2001). Therapeutically administered APC can utilize residual EPCR as a coreceptor in signaling to achieve protection from severe sepsis (G. R. Bernard et al., N Engl J Med, 344: 699, 2001). In the escalation of sepsis syndrome, thrombin is unlikely to reproduce endothelium-restricted, protective PAR1 signaling of APC, because thrombin's targets include PARs on a number of cell types that are activated in the context of microthrombotic organ dysfunction. Unexpectedly, MCP-1 was identified as a gene that is selectively upregulated by APC-dependent PAR1 signaling in endothelial cells. In systemically and locally induced sepsis models, MCP-1 is protective (D. A. Zisman et al., J Clin Invest, 99: 2832, 1997; C. L. Bone-Larson et al., Am J Pathol, 157: 1177, 2000). In addition to the direct endothelial protective functions of APC, local MCP-1 induction by APC can promote indirect anti-inflammatory effects through an immuno-modulatory chemokine network that controls the host defense in sepsis.

The expression profile for an endothelial cell upregulated by thrombin signaling or unchanged or downregulated by APC-PAR1 signaling is shown in Table 4. Table 4 shows the following unchanged or downregulated by APC-PAR1 signaling and upregulated by thrombin signaling: SH-PTP3, W28170, fructose-6-phosphate,2-kinase/fructose-2,6-bisphosphatase, W28616, BID, NF-KB2, thrombospondin-1,6-phosphofructo-2-kinase/fructose-2,6-bisphosphatase, neurofibromatosis 2 tumor suppressor, cationic amino acid transporter 2, W28616, NF-kappa-B p65delta3, TTF-I interacting peptide 12, ATRC2, alpha adducin, sortilin, p53 cellular tumor antigen, W28257, hERRa1, G protein-coupled receptor, EGFRBP-GRB2, Pax8, SH-PTP3, 5T4, bc1-xL, disintegrin-metalloprotease, C1q-related factor, cyclin D1, A1743606, myosin-IXb, GNS, ISLR, Stat2, stem cell factor, c-ets-1, usurpin-beta, chromosome 5q21-22, clone-A3-A, thrombospondin, ELL2, dual-specificity protein phosphatase, vitronectin receptor alpha subunit, PCTAIRE-2, follistatin, ets-2, ELL2, utrophin, C8FW phosphoprotein, PCTAIRE-2, fra-2, MINOR, GADD34, IL8, SSR alpha, neuron derived orphan receptor, CGGBP, nma, jun-B, epsilon tyrosine phosphatase, GRO-beta, CtIP, PRDII-BF1, vascular endothelial growth factor, stanniocalcin-related protein, CL 100, BMP-2A, NF-ATc, mrg1, jun-B, VEGF, STC, and ATF3; these genes are referred to herein as genes downregulated by anti-inflammatory APC-PAR1 signaling.

The identified gene products can be used to develop specific activators or PAR1 agonists to reduce systemic inflammation in a mammalian subject or to treat sepsis in a mammalian subject. Gene expression profiles are provided for endothelial cells in an anti-inflammatory state as a result of APC/PAR1 signaling. Table 1 shows the following genes that are unchanged by PAR2 agonist signaling and upregulated by PAR1 agonist signaling or APC signaling in endothelial cells: MCP-1, qe82d12.x1, RACH1, PAC747L4, T-plastin, endothelin receptor type B-like protein, VEGF, and wx60d10.x1. These genes are referred to herein as PAR1 agonist/APC upregulated endothelial cell expression profile genes.

Materials and Methods

Reporter Gene Assay. The human EPCR cDNA was PCR amplified and subcloned into pEGFP-N1 (Clontech) introducing a linker of two Gly residues between EPCR and the C-terminal EGFP protein. A mutant EPCR construct that lacks PC/APC binding activity was generated by replacing Tyr154 with Ala (P. C. Liaw, et al., J Biol Chem, 276: 8364, 2001) using oligonucleotide directed mutagenesis. A palmitoylation resistant mutant of EPCR was generated by replacing the single cytoplasmic Cys221 with Ser. The coding sequences of all constructs were confirmed by DNA sequencing. Thrombin, APC, hirudin, the PAR1 agonist peptide TFLLRNPNDK, the PAR2 agonist peptide SLIGRL, and monoclonal anti PAR1 antibodies ATAP2 (used at 10 μg/ml) and WEDE15 (used at 20 μg/ml) were as described previously (M. Riewald, W. Ruf, Proc Natl Acad Sci USA, 98: 7742, 2001; P. J. O'Brien et al., J Biol Chem, 275: 13502, 2000; M. Riewald et al., Blood, 97: 3109, 2001). APC was active site modified with 0.5 mM H-D-Phe-L-Phe-Arg chloromethylketone (Bachem Bioscience Inc.) and the absence of residual APC activity and free chloromethyl ketone was confirmed following extensive dialysis. Lysophosphatidic acid (LPA) was obtained from Sigma. The murine PAR deficient M6-11 cell line was co-transfected with human PAR1 or PAR2 and an egr-1 promoter luciferase reporter construct Overnight serum starved cells were incubated with 20 nM APC, 10 μM PAR1 agonist TFLLRNPNDK, 100 μM PAR2 agonist SLIGRL, or 5 nM thrombin (IIa) for 5 h. All experiments involving APC contained 100 nM hirudin to exclude thrombin signaling. Cleavage blocking antibodies against PAR1 were added 10 min prior to the agonists where indicated.

MAP kinase phosphorylation in endothelial cells. Primary human umbilical vein endothelial cells (HUVEC) were incubated for 5 hours with fresh M199 containing 2% serum prior to agonist stimulation as above. Erk1/2 phosphorylation was analyzed by Western blotting after 6 min of stimulation. To block APC binding to EPCR, 100 nM chloromethylketone modified APC (APC-CK) was added. To block PAR1 cleavage, anti PAR1 antibodies were added 10 minutes prior to agonist stimulation. MAP kinase phosphorylation was quantified by laser densitometry of blots and statistical significance was determined by t-test.

Microarray Analysis. Confluent human umbilical vein endothelial cells HUVEC-C (ATCC CRL 1730) were serum deprived for 5 h in M199 medium, 2 mM CaCl₂, 10 mM Hepes, followed by stimulation with 10 μM TFLLRNPNDK, 100 μM SLIGRL, 10 nM APC/100 nM hirudin, or 5 nM thrombin (IIa) for 90 min at 37° C. Total RNA was isolated followed by reverse transcription. Purified double stranded cDNA was in vitro transcribed in the presence of biotinylated dUTP and dCTP, fragmented cRNA was hybridized to the HG-U95Av2 array (Affymetrix, Santa Clara, Calif.), followed by staining with streptavidin-phycoerythrin. Arrays were scanned and analyzed using GeneChip3.1 software. Three independent experiments analyzing gene induction by PAR1, PAR2, or APC yielded 12 data sets from high density microarrays with 9 comparisons between the agonists and control. An average fold-induction for the three independent experiments was obtained by calculating the means of the logarithm of the individual fold-increases for each agonist. To eliminate non-expressed and highly variable genes, the following criteria were set for genes shown: (1) The gene's expression level had to be scored “Present” by the GeneChip software algorithm in more than two of the 12 data sets. (2) GeneChip3.1 comparison analysis of stimulated versus control had to score “Increased” in more than one out of the 9 comparisons. (3) The ratio of the average fold induction for all agonists and the mean of the standard deviations for the inductions by each agonist had to be larger than 1 for inclusion in FIG. 2 or larger than 0.5 for the evaluation of PAR1 and PAR2 selective responses in the larger gene set.

TaqMan Real Time PCR. For TaqMan (Applied Biosystems) real-time RT-PCR 4 μg total cellular RNA from stimulated primary HUVEC was reverse transcribed followed by RNAse treatment using the Superscript Kit (Invitrogen). TaqMan probes were custom designed for genes of interests. All samples were normalized with human GAPDH probes and primers obtained from Applied Biosystems.

TABLE 1
Gene induction profiles determined by microarray analysis of endothelial cells
stimulated with APC, PAR1, or PAR2 agonists. The first set shows genes that were
induced with high reproducibility by the three agonists. Selected genes from a larger
gene set with higher variability between repeat experiments is shown below, grouped
according to PAR1 and PAR2 selective expression. Certain genes were not induced
by APC, but were induced by PAR1 or PAR2 agonists.

| | Average | | |
|---|---|---|---|
| | Induction | | |
| Gene | PAR1 | PAR2 | APC |
| Genes represented in FIG. 2 A-C | | | |
| L13740/FEATURE=/DEFINITION=HUMTR3A Human TR3 orphan receptor mRNA | 4.8 | 5.4 | 3.3 |
| L13740/FEATURE=/DEFINITION=HUMTR3A Human TR3 orphan receptor mRNA | 4.7 | 5.4 | 3.2 |
| Cluster Incl M60278:Human heparin-binding EGF-like growth factor mRNA | 3.6 | 3.9 | 3.5 |
| U12767/FEATURE=/DEFINITION=HSU12767 Human mitogen induced nuclear orphan receptor | 4.9 | 3.3 | 2.6 |
| (MINOR) | | | |
| M29039/FEATURE=cds/DEFINITION=HUMJUNCAA Human transactivator (jun-B) gene | 2.8 | 2.8 | 4.9 |
| M60721/FEATURE=mRNA/DEFINITION=HUMHB24 Human homeobox gene | 2.8 | 3.7 | 2.3 |
| U15932/FEATURE=/DEFINITION=HSU15932 Human dual-specificity protein phosphatase | 2.9 | 3.3 | 1.8 |
| Cluster Incl J02931:Human placental tissue factor (two forms) mRNA | 3.0 | 2.7 | 2.1 |
| M22489/FEATURE=/DEFINITION=HUMBMP2A Human bone morphogenetic protein 2A | 2.3 | 3.2 | 2.4 |
| (BMP-2A) | | | |
| Cluster Incl X75918:H.sapiens mRNA for NOT | 2.3 | 3.3 | 2.2 |
| Cluster Incl U75697:Human transcription regulator RPD3-2B mRNA | 2.7 | 2.6 | 2.3 |
| Cluster Incl U12767:Human mitogen induced nuclear orphan receptor (MINOR) | 2.4 | 3.4 | 1.7 |
| Cluster Incl M92843:H.sapiens zinc finger transcriptional regulator mRNA, | 2.0 | 3.4 | 1.8 |
| Cluster Incl M30474:Human kidney gamma-glutamyl transpeptidase type II mRNA | 2.7 | 2.1 | 2.3 |
| L19871/FEATURE=/DEFINITION=HUMATF3X Human activating transcription factor 3 (ATF3) | 2.5 | 2.6 | 1.8 |
| Cluster Incl M22489:Human bone morphogenetic protein 2A (BMP-2A) | 2.4 | 2.9 | 1.6 |
| Cluster Incl X63741:H.sapiens pilot mRNA | 1.9 | 3.0 | 1.9 |
| X68277/FEATURE=cds/DEFINITION=HSCL100 H.sapiens CL 100 mRNA for protein tyrosine | 2.1 | 2.8 | 1.9 |
| phosphatase | | | |
| Cluster Incl M28211:Homo sapiens GTP-binding protein (RAB4) mRNA | 2.0 | 2.7 | 1.8 |
| Cluster Incl A1288757:qm11h01.x1 Homo sapiens cDNA, 3′ end/clone=IMAGE-1881553 | 1.8 | 2.3 | 2.3 |
| Cluster Incl M36820:Human cytokine (GRO-beta) mRNA | 2.1 | 1.6 | 2.6 |
| Cluster Incl AL031432:Human DNA sequence from clone 465N24 on chromosome 1p35.1-36.13. | 1.9 | 2.6 | 1.7 |
| Cluster Incl Z48054:H.sapiens mRNA for peroxisomal targeting signal 1 (SKL type) receptor | 2.0 | 1.9 | 2.2 |
| Cluster Incl D15050:Human mRNA for transcription factor AREB6 | 2.1 | 1.7 | 2.2 |
| Cluster Incl X89066:H.sapiens mRNA for TRPC1 protein | 2.6 | 2.2 | 0.9 |
| L05072/FEATURE=expaned_cds/DEFINITION=HUMIFNRF1A Homo sapiens interferon | 1.9 | 2.0 | 1.7 |
| regulatory factor 1 gene | | | |
| Cluster Incl X51345:Human jun-B mRNA for JUN-B protein | 1.8 | 1.8 | 2.1 |
| Cluster Incl D78579:Homo sapiens mRNA for neuron derived orphan receptor | 2.6 | 1.4 | 1.5 |
| Cluster Incl J04076:Human early growth response 2 protein (EGR2) mRNA | 2.0 | 2.0 | 1.5 |
| Cluster Incl AB014569:Homo sapiens mRNA for K1AA0669 protein | 1.3 | 2.0 | 2.1 |
| Cluster Incl AJ005821:Homo sapiens mRNA for X-like 1 protein | 2.4 | 1.6 | 1.4 |
| Cluster Incl Y00630:Human mRNA for Arg-Serpin (plasminogen activator-inhibitor 2, PAI-2) | 2.2 | 1.7 | 1.5 |
| Cluster Incl M31516:Human decay-accelerating factor mRNA | 1.7 | 1.9 | 1.5 |
| X94216/FEATURE=cds /DEFINITION=HSVEGFC H.sapiens mRNA for VEGF-C protein | 1.4 | 2.1 | 1.7 |
| Cluster Incl AL023584:Human DNA sequence from clone 67K17 on chromosome 6q24.1-24.3. | 1.8 | 2.0 | 1.3 |
| Cluster Incl M28225:Human JE gene encoding a monocyte secretory protein [= MCP-1] | 2.1 | 1.1 | 2.0 |
| Cluster Incl D86181:Homo sapiens DNA for galactocerebrosidase | 1.9 | 1.5 | 1.6 |
| Cluster Incl AF017786:Homo sapiens phosphatidic acid phosphohydrolase homolog (Dri42) | 1.6 | 1.6 | 1.8 |
| U04636/FEATURE=mRNA /DEFINITION=HSU04636 Human cyclooxygenase-2 (hCox-2) gene | 1.3 | 1.3 | 2.3 |
| Guanine Nucleotide Exchange Factor 2 | 1.4 | 1.4 | 2.1 |
| Cluster Incl AF039843:Homo sapiens Sprouty 2 (SPRY2) mRNA | 1.6 | 1.8 | 1.5 |
| Cluster Incl U08015:Human NF-ATc mRNA | 1.4 | 2.0 | 1.4 |
| M59465/FEATURE=/DEFINITION=HUMA20 Human tumor necrosis factor alpha inducible | 1.4 | 1.3 | 2.1 |
| protein A20 mRNA | | | |
| U43142/FEATURE=/DEFINITION=HSU43142 Human vascular endothelial growth factor related | 1.4 | 1.9 | 1.4 |
| protein VRP | | | |
| Cluster Incl U79259:Human clone 23945 mRNA | 1.5 | 1.7 | 1.5 |
| Cluster Incl AF068197:Homo sapiens BCE-1 mRNA | 1.5 | 1.8 | 1.4 |
| Cluster Incl AF054182:Homo sapiens mitochondrial processing peptidase beta-subunit mRNA | 1.8 | 1.3 | 1.5 |
| Cluster Incl AL050078:Homo sapiens mRNA; cDNA DKFZp566G0746 | 1.6 | 1.6 | 1.3 |
| Cluster Incl J04111:Human c-jun proto oncogene (JUN) | 1.7 | 1.5 | 1.4 |
| M69043/FEATURE=/DEFINITION=HUMMAD3A Homo sapiens MAD-3 mRNA encoding 1kB- | 1.4 | 1.4 | 1.8 |
| like activity | | | |
| Cluster Incl AF050110:Homo sapiens TGFb inducible early protein and early growth response | 1.7 | 1.4 | 1.4 |
| protein alpha genes | | | |
| S81439/FEATURE=/DEFINITION=S81439 EGR alpha=early growth response gene alpha | 1.8 | 1.4 | 1.3 |
| Cluster Incl A1670788:tz10c02.x1 Homo sapiens cDNA, 3′ end/clone=IMAGE-2288162 | 1.5 | 1.4 | 1.6 |
| Cluster Incl A8022718:Homo sapiens m.RNA for DEPP (decidual protein induced by progesterone) | 1.3 | 2.0 | 0.9 |
| Cluster Incl U25997:Homo sapiens stanniocalcin precursor (STC) mRNA | 1.6 | 1.4 | 1.2 |
| Cluster Incl AB004066:Homo sapiens mRNA for DEC1 | 1.5 | 1.3 | 1.5 |
| Cluster Incl U07802:Human Tis1 1d gene | 1.5 | 1.6 | 1.2 |
| Cluster Incl AL096858:Novel human gene mapping to chomosome 1 | 1.3 | 1.5 | 1.4 |
| M26683/FEATURE=/DEFINITION=HUMIFNIND Human interferon gamma treatment inducible | 1.6 | 1.0 | 1.6 |
| mRNA | | | |
| Cluster Incl AF022375:Homo sapiens vascular endothelial growth factor mRNA | 1.5 | 1.3 | 1.4 |
| Cluster Incl U81607:Homo sapiens gravin mRNA | 1.4 | 1.3 | 1.5 |
| Cluster Incl L43821:Homo sapiens enhancer of filamentation (HEF1) mRNA | 1.4 | 1.2 | 1.6 |
| Cluster Incl AF045451:Homo sapiens transcriptional regulatory protein p54 | 1.5 | 1.4 | 1.2 |
| Cluster Incl AF078077:Homo sapiens growth arrest and DNA-damage-inducible protein | 1.3 | 1.3 | 1.5 |
| GADD45beta | | | |
| Cluster Incl D50917:Human mRNA for KIAA0127 gene | 1.4 | 1.3 | 1.2 |
| Cluster Incl A1985272:ws06b05.x1 Homo sapiens cDNA, 3′ end/clone=IMAGE-2496369 | 1.3 | 1.4 | 1.1 |
| S81914/FEATURE=/DEFINITION=S81914 IEX-1=radiation-inducible immediate-early gene | 1.3 | 1.2 | 1.3 |
| Cluster Incl Z25821:H.sapiens gene for mitochondrial dodecenoyl-CoA delta-isomerase | 1.1 | 1.7 | 1.0 |
| Cluster Incl W27545:32c4 Homo sapiens cDNA | 1.2 | 1.2 | 1.2 |
| Cluster Incl U59863:Human TRAF-interacting protein I-TRAF mRNA | 1.3 | 1.1 | 1.2 |
| Genes with higher variability (not represented in FIG.2) | | | |
| PAR1 enhanced (<1.3 fold with PAR2 agonist) | | | |
| Cluster Incl A1186701:qe82d12.x1 Homo sapiens cDNA, 3′ end/clone=IMAGE-1745495 | 1.9 | 1.1 | 1.6 |
| Cluster Incl U35735:Human RACH1 (RACH1) mRNA | 1.8 | 1.1 | 1.2 |
| Cluster Incl AL035297:H.sapiens gene from PAC 747L4 | 1.7 | 1.0 | 1.4 |
| Cluster Incl M22299:Human T-plastin polypeptide mRNA | 1.6 | 1.2 | 2.0 |
| Cluster Incl U87460:Human putative endothelin receptor type B-like protein mRNA | 1.4 | 0.9 | 1.8 |
| AF024710/FEATURE=/DEFINITION=AF024710 Homo sapiens vascular endothelial growth | 1.4 | 1.1 | 1.1 |
| factor (VEGF) | | | |
| Cluster Incl A1953789:wx69d10.x1 Homo sapiens cDNA, 3′ end/clone=IMAGE-2548915 | 1.3 | 0.9 | 1.4 |
| PAR2 enhanced (<1.3 fold with PAR1 agonist) | | | |
| Cluster Incl A1038821:ox96d03.x1 Homo sapiens cDNA, 3′ end/clone=IMAGE-1664165 | 1.2 | 2.0 | 1.3 |
| Cluster Incl U65093:Human msg1-related gene 1 (mrg1) mRNA | 1.3 | 1.7 | 1.0 |
| Cluster Incl Y13115:Homo sapiens mRNA for serine/threonine protein kinase SAK | 1.2 | 1.6 | 1.0 |
| Cluster Incl W26851:17b12 Homo sapiens cDNA | 1.2 | 1.6 | 1.1 |
| Cluster Incl U53831:Homo sapiens interferon regulatory factor 7B | 1.1 | 1.5 | 1.3 |
| Cluster Incl U27655:Human RGP3 mRNA | 1.2 | 1.5 | 1.2 |
| Cluster Incl U58334:Human Bcl2, p53 binding protein Bbp/53BP2 (BBP/53BP2) mRNA | 1.1 | 1.5 | 1.1 |
| Cluster Incl AC004475:Homo sapiens chromosome 19, cosmid F23858 | 1.2 | 1.4 | 1.2 |
| Cluster Incl L38696:Homo sapiens autoantigen p542 mRNA | 1.0 | 1.3 | 1.1 |
| Cluster Incl AL049354:Homo sapiens mRNA; cDNA DKFZp566E183 | 1.1 | 1.3 | 0.9 |
| Cluster Incl Z25821:H.sapiens gene for mitochondrial dodecenoyl-CoA delta-isomerase | 1.1 | 1.7 | 1.0 |
| Genes not induced by APC (<1.3 fold with APC) | | | |
| Cluster Incl AB028069:Homo sapiens mRNA for activator of S phase Kinase | 2.0 | 2.0 | 1.2 |
| Cluster Incl U85267:Homo sapiens down syndrome candidate region 1 (DSCR1) gene | 1.6 | 2.0 | 0.9 |
| Cluster Incl U78082:Human RNA polymerase transcriptional regulation mediator (h-MED6) | 1.9 | 1.3 | 1.1 |
| mRNA | | | |
| Cluster Incl D38537:Human mRNA for protoporphyrinogen oxidase | 1.3 | 1.6 | 1.0 |
| M83667/FEATURE=mRNA/DEFINITION=HUMNFIL6BA Human NF-IL6-beta protein | 1.4 | 1.3 | 1.1 |

TABLE 2
Selected protective genes (from FIG. 2A-C) induced in endothelial cells by PAR
activation and APC signaling. Average fold inductions with the different agonists and
OMIM abbreviations and GeneBank accession numbers are given. IAP and the Bc12
homologue A1 did not meet the stringent criteria for inclusion in FIG. 2. These genes
were reproducibly upregulated in the gene chip experiment and had been previously
found induced upon prolonged APC stimulation (D. E. Joyce, et al., J Biol Chem,
276: 11199, 2001).

| OMIM | Accession | Gene | PAR1 | PAR2 | APC |
|---|---|---|---|---|---|
| AKAP12 | U81607 | A-KINASE ANCHOR PROTEIN 12, GRAVIN | 1.39 | 1.28 | 1.47 |
| DUSP1 | X68277 | DUAL-SPECIFICITY PHOSPHATASE 1 | 2.05 | 2.78 | 1.94 |
| DUSP5 | U15932 | DUAL-SPECIFICITY PHOSPHATASE 5 | 2.93 | 3.27 | 1.76 |
| GADD45B | AF078077 | GROWTH ARREST- AND DNA | 1.26 | 1.27 | 1.50 |
| | | DAMAGE-INDUCIBLE GENE GADD45, BETA | | | |
| IER3 | S81914 | IMMEDIATE-EARLY RESPONSE 3 | 1.30 | 1.23 | 1.29 |
| NAB1 | AF045451 | NGFIA-BINDING PROTEIN | 1.49 | 1.38 | 1.24 |
| NFKBIA | M69043 | NUCLEAR FACTOR OF KAPPA LIGHT CHAIN | 1.38 | 1.40 | 1.79 |
| | | GENE | | | |
| | | ENHANCER IN B CELLS INHIBITOR, ALPHA | | | |
| TNFAIP3 | M59465 | TUMOR NECROSIS FACTOR-ALPHA- | 1.39 | 1.32 | 2.10 |
| | | INDUCED PROTEIN 3, A20 | | | |
| ZFP36 | M92843 | ZINC FINGER PROTEIN 36, TRISTETRAPROLIN | 1.98 | 3.38 | 1.83 |
| | | (TTP) | | | |
| BCL2A1 | U27467 | BCL2 RELATED PROTEIN A1, BFL-1 | 2.04 | 2.57 | 1.87 |
| BIRC3 | U45878 | HUMAN INHIBITOR OF APOPTOSIS PROTEIN | 2.39 | 1.41 | 1.79 |
| | | (IAP) 1 | | | |

### Example 2

The methods and compositions identify unique gene targets for anti-inflammatory therapy utilizing the endothelial cell APC/EPCR/PAR1 signaling pathway. Genes that are down-regulated by anti-inflammatory APC/EPCR/PAR1 signaling are shown in Table 3. Thrombin signaling through PAR1 has opposing effects to APC signaling and promotes inflammation. Table 3 indicates genes that are up-regulated or induced by thrombin, but not by APC/EPCR/PAR1 signaling.

The identified gene products can be used as targets to develop specific inhibitors to reduce systemic inflammation in a mammalian subject or to treat sepsis in a mammalian subject. The expression profile for an endothelial cell upregulated by thrombin signaling or unchanged or downregulated by APC-PAR1 signaling is shown. Table 3 shows the following unchanged or downregulated by APC-PAR1 signaling and upregulated by thrombin signaling: SH-PTP3, W28170, fructose-6-phosphate,2-kinase/fructose-2,6-bisphosphatase, W28616, BID, NF-KB2, thrombospondin-1,6-phosphofructo-2-kinase/fructose-2,6-bisphosphatase, neurofibromatosis 2 tumor suppressor, cationic amino acid transporter 2, W28616, NF-kappa-B p65delta3, TTF-I interacting peptide 12, ATRC2, alpha adducin, sortilin, p53 cellular tumor antigen, W28257, hERRa1, G protein-coupled receptor, EGFRBP-GRB2, Pax8, SH-PTP3, 5T4, bc1-xL, disintegrin-metalloprotease, C1q-related factor, cyclin D1, AI743606, myosin-IXb, GNS, ISLR, Stat2, stem cell factor, c-ets-1, usurpin-beta, chromosome 5q21-22, clone-A3-A, thrombospondin, ELL2, dual-specificity protein phosphatase, vitronectin receptor alpha subunit, PCTAIRE-2, follistatin, ets-2, ELL2, utrophin, C8FW phosphoprotein, PCTAIRE-2, fra-2, MINOR, GADD34, IL8, SSR alpha, neuron derived orphan receptor, CGGBP, nma, jun-B, epsilon tyrosine phosphatase, GRO-beta, CtIP, PRDII-BF1, vascular endothelial growth factor, stanniocalcin-related protein, CL 100, BMP-2A, NF-ATc, mrg1, jun-B, VEGF, STC, and ATF3; these genes are referred to herein as genes downregulated by anti-inflammatory APC-PAR1 signaling.

TABLE 3
Protective genes induced in endothelial cells by APC-PAR1 signaling/ down regulation
and thrombin signaling. Average fold inductions with apc or thrombin;
OMIM abbreviations and GeneBank accession numbers are indicated.

| | Induction (log 2) by | |
|---|---|---|
| GENEBANK | APC | Thrombin |
| D13540/FEATURE=/DEFINITION=HUMSHPTP3 Homo sapiens SH-PTP3 mRNA | −1.33 | 0.65 |
| for protein-tyrosine phosphatase, complete cds | | |
| Cluster Incl W28170:43a12 Homo sapiens cDNA/gb=W28170/gi=1308118 | −1.07 | 0.15 |
| /ug=Hs.181165/len=912 | | |
| Cluster Incl AB012229:Homo sapiens gene for fructose-6-phosphate,2-kinase/fructose-2, | −1.07 | 0.25 |
| 6-bisphosphatase, partial cds/cds=(0,530)/gb=AB012229/gi=2982725/ug=Hs.101313 | | |
| /len=531 | | |
| Cluster Incl W28616:49b9 Homo sapiens cDNA/gb=W28616/gi=1308564 | −0.97 | 0.30 |
| /ug=Hs.74335/len=840 | | |
| Cluster Incl AF042083:Homo sapiens BH3 interacting domain death agonist (BID) | −0.87 | 0.20 |
| mRNA, complete cds/cds=(140,727)/gb=AF042083/gi=3540246/ug=Hs.172894 | | |
| /len=1105 | | |
| U20816/FEATURE=mRNA#1/DEFINITION=HSU20816 Human nuclear factor | −0.77 | 0.70 |
| kappa-B2 (NF-KB2) gene, partial cds | | |
| U12471/FEATURE=cds#2/DEFINITION=HSU12471 Human thrombospondin-1 gene, | −0.77 | 0.70 |
| partial cds | | |
| Cluster Incl D49817:Homo sapiens mRNA for 6-phosphofructo-2-kinase/fructose-2, 6- | −0.73 | 0.20 |
| bisphosphatase, complete cds/cds=(18,1580)/gb=D49817/gi=1468914/ug=Hs.101313 | | |
| /len=1756 | | |
| Neurofibromatosis 2 Tumor Suppressor | −0.73 | 0.55 |
| Cluster Incl D29990:Human mRNA for cationic amino acid transporter 2, complete cds | −0.70 | 0.75 |
| /cds=(0,1976)/gb=D29990/gi=484049/ug=Hs.153985/len=1977 | | |
| Cluster Incl W28616:49b9 Homo sapiens cDNA/gb=W28616/gi=1308564 | −0.67 | −0.05 |
| /ug=Hs.74335/len=840 | | |
| U33838/FEATURE=/DEFINITION=HSU33838 Human NF-kappa-B p65delta3 | −0.67 | 0.10 |
| mRNA, spliced transcript lacking exons 6 and 7, partial cds | | |
| Cluster Incl AF000421:Homo sapiens TTF-1 interacting peptide 12 mRNA, partial cds | −0.60 | 0.20 |
| /cds=(0,614)/gb=AF000421/gi=2183080/ug=Hs.159219/len=689 | | |
| Cluster Incl U76368:Human cationic amino acid transporter-2A (ATRC2) mRNA, | −0.60 | 0.75 |
| complete cds/cds=(194,2167)/gb=U76368/gi=2252785/ug=Hs.153985/len=2185 | | |
| Cluster Incl L07261:Human alpha adducin mRNA, partial cds including alternate exons | −0.57 | 0.10 |
| A and B/cds=UNKNOWN/gb=L07261/gi=178087/ug=Hs.183706/len=2343 | | |
| Cluster Incl X98248:H.sapiens mRNA for sortilin/cds=(21,2522)/gb=X98248 | −0.53 | −0.15 |
| /gi=1834494/ug=Hs.104247/len=3723 | | |
| X02469/FEATURE=cds/DEFINITION=HSP53 Human mRNA for p53 cellular tumor | −0.53 | −0.10 |
| antigen | | |
| Cluster Incl W28257:44c1 Homo sapiens cDNA/gb=W28257/gi=1308205 | −0.50 | 0.20 |
| /ug=Hs.239274/len=821 | | |
| L38487/FEATURE=mRNA/DEFINITION=HUMHERRA1 Human estrogen receptor- | −0.50 | 0.10 |
| related protein (hERRa1) mRNA, 3″ end, partial cds | | |
| D38449/FEATURE=/DEFINITION=HUMGPCRAA Human mRNA for G protein- | −0.47 | 0.10 |
| coupled receptor, complete cds | | |
| Cluster Incl M96995:Homo sapiens epidermal growth factor receptor-binding protein | −0.43 | 0.05 |
| GRB2 (EGFRBP-GRB2) mRNA sequence/cds(78,731)/gb=M96995/gi=181975 | | |
| /ug=Hs.239528/len=1109 | | |
| X69699/FEATURE=/DEFINITION=HSPAX8A H.sapiens Pax8 mRNA | −0.43 | 0.10 |
| D13540/FEATURE=/DEFINITION=HUMSHPTP3 Homo sapiens SH-PTP3 mRNA | −0.43 | 0.40 |
| for protein-tyrosine phosphatase, complete cds | | |
| Z29083/FEATURE=cds/DEFINITION=HS5T4OA H.sapiens 5T4 gene for 5T4 | −0.43 | 0.10 |
| Oncofetal antigen | | |
| Z23115/FEATURE=cds/DEFINITION=HSBCLXL H.sapiens bc1-xL mRNA | −0.40 | 0.10 |
| Cluster Incl Z48579:H.sapiens mRNA for disintegrin-metalloprotease (partial) | −0.40 | 0.20 |
| /cds=(0,2075)/gb=Z48579/gi=1616600/ug=Hs.172028/len=2423 | | |
| Cluster Incl AF095154:Homo sapiens C1q-related factor mRNA, complete cds | −0.40 | 0.10 |
| /cds=(13,789)/gb=AF095154/gi=3747096/ug=Hs.134012/len=1284 | | |
| M64349/FEATURE=/DEFINITION=HUMCYCD1 Human cyclin D (cyclin D1) | −0.37 | 0.00 |
| mRNA, complete cds | | |
| Cluster Incl A1743606:wg51108.x1 Homo sapiens cDNA, 3 end/clone=IMAGE- | −0.37 | −0.05 |
| 2368647/clone_end=3″ /gb=A1743606/gi=5111894/ug=Hs.5947/len=742Δ | | |
| Cluster Incl U42391:Human myosin-IXb mRNA, complete cds/cds=(0,6068) | −0.33 | −0.15 |
| /gb=U42391/gi=1147782/ug=Hs.159629/len=6069 | | |
| Cluster Incl Z12173:H.sapiens GNS mRNA encoding glucosamine-6-sulphatase | −0.33 | 0.60 |
| /cds=(87,1745)/gb=Z12173/gi=31866/ug=Hs.2703/len=2379 | | |
| Cluster Incl AB003184:Homo sapiens mRNA for ISLR, complete cds/cds=(98,1384) | −0.30 | 0.05 |
| /gb=AB003184/gi=2554603/ug=Hs.102171/len=2110 | | |
| U18671/FEATURE=mRNA/DEFINITION=HSU18671 Human Stat2 gene, complete | −0.30 | 0.05 |
| cds | | |
| M59964/FEATURE=/DEFINITION=HUMSCF Human stem cell factor mRNA, | −0.27 | 0.75 |
| complete cds | | |
| X14798/FEATURE=cds#1/DEFINITION=HSCETS1 Human DNA for c-ets-1 proto- | −0.27 | 0.75 |
| oncogene | | |
| Cluster Incl AF015451:Homo sapiens Usurpin-beta mRNA, complete cds/cds=(0,1388) | −0.23 | 0.45 |
| /gb=AF015451/gi=3133282/ug=Hs.195175/len=1389 | | |
| Cluster Incl AB002450:Homo sapiens mRNA from chromosome 5q21-22, clone-A3-A | −0.20 | 0.45 |
| /cds=UNKNOWN/gb=AB002450/gi=2943816/ug=Hs.169400/len=1053 | | |
| X14787/FEATURE=cds/DEFINITION=HSTS Human mRNA for thrombospondin | −0.20 | 0.40 |
| Cluster Incl U88629:Human RNA polymerase II elongation factor ELL2, complete cds | −0.20 | 0.70 |
| /cds=(0,1922)/gb=U88629/gi=1946346/ug=Hs.173334/len=1923 | | |
| U15932/FEATURE=/DEFINITION=HSU15932 Human dual-specificity protein | −0.20 | 0.60 |
| phosphatase mRNA, complete cds | | |
| M14648/FEATURE=/DEFINITION=HUMVTNR Human cell adhesion protein | −0.17 | 0.60 |
| (vitronectin) receptor alpha subunit mRNA, complete cds | | |
| X66360/FEATURE=cds/DEFINITION=HSSTHPKC H.sapiens mRNA PCTAIRE-2 | −0.17 | 0.65 |
| for serine/threonine protein kinase | | |
| Cluster Incl M19481:Human follistatin gene/cds=(0,953)/gb=M19481/gi=182720 | −0.13 | 1.30 |
| /ug=Hs.9914/len=954 | | |
| J04102/FEATURE=/DEFINITION=HUMETS2A Human erythroblastosis virus | −0.10 | 0.50 |
| oncogene homolog 2 (ets-2) mRNA, complete cds | | |
| U88629/FEATURE=cds/DEFINITION=HSU88629 Human RNA polymerase II | −0.10 | 0.75 |
| elongation factor ELL2, complete cds | | |
| Cluster Incl X69086:H.sapiens mRNA for utrophin/cds=(0,10301)/gb=X69086 | −0.10 | 0.60 |
| /gi=34811/ug=Hs.104252/len=10302 | | |
| Cluster Incl AJ000480:Homo sapiens mRNA for C8FW phosphoprotein/cds=(0,674) | −0.10 | 1.15 |
| /gb=AJ000450/gi=2274958/ug=Hs.143513/len=675 | | |
| Cluster Incl X66360:H.sapiens mRNA PCTAIRE-2 for serine/threonine protein kinase | −0.07 | 0.35 |
| /cds=(69,1640)/gb=X66360/gi=36616/ug=Hs.123063/len=1738 | | |
| Cluster Incl X16706:Human fra-2 mRNA/cds=(3,983)/gb=X16706/gi=31464 | −0.07 | 0.50 |
| /ug=Hs.155210/len=1007 | | |
| U12767/FEATURE=/DEFINITION=HSU12767 Human mitogen induced nuclear | −0.07 | 1.15 |
| orphan receptor (MINOR) mRNA, complete cds | | |
| Cluster Incl U83981:Homo sapiens apoptosis associated protein (GADD34) mRNA, | −0.07 | 0.50 |
| complete cds/cds=(222,2246)/gb=U83981/gi=3258617/ug=Hs.76556/len=2331 | | |
| M28130/FEATURE=mRNA/DEFINITION=HUMIL8A Human interleukin 8 (IL8) | −0.07 | 0.40 |
| gene, complete cds | | |
| Cluster Incl Z12830:H.sapiens mRNA for SSR alpha subunit/cds=(29,889)/gb=Z12830 | −3.7E- | 0.40 |
| /gi=551637/ug=Hs.76152/len=974 | 17 | |
| Cluster Incl D78579:Homo sapiens mRNA for neuron derived orphan receptor, complete | 0.00 | 1.55 |
| cds/cds=(731,2611)/gb=D78579/gi=1651190/ug=Hs.80561/len=3802 | | |
| Cluster Incl AF094481:Homo sapiens trinucleotide repeat DNA binding protein p20- | 0.00 | 0.50 |
| CGGBP (CGGBP) gene, complete cds/cds=(479,982)/gb=AF094481/gi=4140681 | | |
| /ug=Hs.107587/len=1043 | | |
| Cluster Incl U23070:Human putative transmembrane protein (nma) mRNA, complete | 0.00 | 1.10 |
| cds/cds=(372,1154)/gb=U23070/gi=1262172/ug=Hs.78776/len=1521 | | |
| M29039/FEATURE=cds/DEFINITION=HUMJUNCAA Human transactivator (jun-B) | 0.00 | 0.60 |
| gene; complete cds | | |
| Tyrosine Phosphatase, Epsilon | 0.03 | 1.40 |
| Cluster Incl M36820:Human cytokine (GRO-beta) mRNA, complete cds/cds=(74,397) | 0.03 | 0.35 |
| /gb=M36820/gi=183628/ug=Hs.75765/len=1110 | | |
| Cluster Incl U72066:Homo sapiens CtBP interacting protein CtIP (CtIP) mRNA, | 0.07 | 0.70 |
| complete cds/cds=(299,2992)/gb=U72066/gi=1730320/ug=Hs.29287/len=3237 | | |
| Cluster Incl X51435:Human PRDII-BF1 gene for a DNA-binding protein | 0.07 | 0.85 |
| /cds=(324,8477)/gb=X51435/gi=38017/ug=Hs.306/len=9020 | | |
| Cluster Incl AF022375:Homo sapiens vascular endothelial growth factor mRNA, | 0.07 | 0.75 |
| complete cds/cds=(701,1276)/gb=AF022375/gi=3719220/ug=Hs.73793 /len=3154 | | |
| Cluster Incl AF098462:Homo sapiens stanniocalcin-related protein mRNA, complete cds | 0.07 | 1.00 |
| /cds=(134,1042)/gb=AF098462/gi=4050037/ug=Hs.155223/len=2380 | | |
| X68277/FEATURE=cds/DEFINITION=HSCL100 H.sapiens CL 100 mRNA for | 0.07 | 1.75 |
| protein tyrosine phosphatase | | |
| Cluster Incl M22489:Human bone morphogenetic protein 2A (BMP-2A) mRNA | 0.10 | 0.90 |
| /cds=(323,1513)/gb=M22489/gi=179501/ug=Hs.73853/len=1547 | | |
| Cluster Incl U08015:HumanNF-ATc mRNA, complete cds/cds=(239,2389) | 0.10 | 0.55 |
| /gb=U08015/gi=500631/ug=Hs.96149/len=2743 | | |
| Cluster Incl U65093:Human msg1-related gene 1 (mrg1) mRNA, complete cds | 0.13 | 0.95 |
| /cds=(199,840)/gb=U65093/gi=1853998/ug=Hs.82071/len=899 | | |
| Cluster Incl X51345:Human jun-B mRNA for JUN-B protein/cds=(253,1296) | 0.13 | 0.75 |
| /gb=X51345/gi=34014/ug=Hs.198951/len=1797 | | |
| AF024710/FEATURE=/DEFINITION=AF024710 Homo sapiens vascular endothelial | 0.13 | 0.90 |
| growth factor (VEGF) mRNA, 3″UTR | | |
| Cluster Incl U25997:Homo sapiens stanniocalcin precursor (STC) mRNA, complete cds | 0.13 | 1.10 |
| /cds=(284,1027)/gb=U25997/gi=3006202/ug=Hs.25590/len=3881 | | |
| L19871/FEATURE=/DEFINITION=HUMATF3X Human activating transcription | 0.20 | 1.10 |
| factor 3 (ATF3) mRNA, complete cds | | |

### Example 3

Coagulation and inflammatory exacerbation in sepsis mediated by thrombin binding and proteolytic activation at PAR1 is counterbalanced by an anti-inflammatory response by the protective protein C pathway mediated by activated protein C (APC) binding to EPCR and PAR1 and proteolytic activation at PAR1. The site of proteolytic cleavage on PAR1 by thrombin and by APC are the same. The PAR1 deficient fibroblast cell-based assay system is useful to screen for PAR1 polypeptides that are activated only by APC proteolytic cleavage and not by thrombin proteolytic cleavage. The PAR1 deficient fibroblast cell-based assay system is also useful to screen for PAR1 polypeptides that are activated only by thrombin proteolytic cleavage and not by APC cleavage. DNA encoding a PAR1 polypeptide is treated by in vitro mutagenesis. DNA encoding a mutagenized PAR1 polypeptide is transfected into PAR1 deficient fibroblast cells and assayed for activation by APC or by thrombin. Mutagenized PAR1 polypeptides activated by APC and not thrombin, or activated by thrombin and not APC, are subjected to iterative rounds of in vitro mutagenesis to isolate DNA encoding PAR1 polypeptides responsive to activation by either protease, APC or thrombin, but not both proteases. PAR1 activation is measured as described above utilizing, for example, by egr-1 promoter/luciferase assay, or by MAP kinase phosphorylation assay, or by microarray analysis of a panel of genes for regulation of gene expression levels.

Transgenic mice expressing a mutant PAR1 polypeptide activated by APC and not thrombin, or activated by thrombin and not APC are useful as a model for testing the pathway for sepsis in a mammalian subject. Such transgenic mice can be more succeptible or less susceptible to inflammation or sepsis depending upon the nature of the change in PAR1 due to the mutation. Test compounds in the form of pharmaceutical compositions administered to transgenic mice that prevent inflammation or sepsis by acting as APC agonists or thrombin antagonists can also contribute to the understanding of the mechanism of sepsis.

All publications and patent documents cited above are hereby incorporated by reference in their entirety for all purposes to the same extent as if each were so individually denoted.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed:

1. A method for identifying a compound which modulates signaling in endothelial cells via a protease activated receptor (PAR)-1 pathway comprising:
contacting a test compound with a cell-based assay system comprising a cell co-expressing endothelial protein C receptor (EPCR) and PAR1 capable of signaling responsiveness to activated protein C (APC);
providing APC to said assay system in an amount selected to be effective to activate signaling; and
detecting an effect of said test compound on PAR1 signaling in said assay system, effectiveness of said test compound in said assay being indicative of said modulation.

2. The method of claim 1, wherein said assay system comprises an endothelial cell.

3. The method of claim 1, wherein said assay system comprises a PAR1 deficient fibroblast cell.

4. The method of claim 3, further comprising measuring a reporter gene activity in said PAR1 deficient fibroblast cell.

5. The method of claim 1, wherein said detecting step further comprises determining a level of gene expression for a panel of genes in endothelial cells subject to activation or repression mediated by APC, EPCR, and PAR1.

6. The method of claim 5, wherein said panel of genes includes at least one of SH-PTP3, W28170, fructose-6-phosphate, 2-kinase/fructose-2,6-bisphosphatase, W28616, BID, NF-KB2, thrombospondin-1,6-phosphofructo-2-kinase/fructose-2,6-bisphosphatase, neurofibromatosis 2 tumor suppressor, cationic amino acid transporter 2, W28616, NF-kappa-B p65delta3, TTF-I interacting peptide 12, ATRC2, alpha adducin, sortilin, p53 cellular tumor antigen, W28257, hERRa1, G protein-coupled receptor, EGFRBP-GRB2, Pax8, SH-PTP3, 5T4, bc1-xL, disintegrin-metalloprotease, C1q-related factor, cyclin D1, A1743606, myosin-IXb, GNS, ISLR, Stat2, stem cell factor, c-ets-1, usurpin-beta, chromosome 5q21-22, clone-A3-A, thrombospondin, ELL2, dual-specificity protein phosphatase, vitronectin receptor alpha subunit, PCTAIRE-2, follistatin, ets-2, ELL2, utrophin, C8FW phosphoprotein, PCTAIRE-2, fra-2, MINOR, GADD34, IL8, SSR alpha, neuron derived orphan receptor, CGGBP, nma, jun-B, epsilon tyrosine phosphatase, GRO-beta, CtIP, PRDII-BF1, vascular endothelial growth factor, stanniocalcin-related protein, CL 100, BMP-2A, NF-ATc, mrg1, jun-B, VEGF, STC, or ATF3.

7. The method of claim 5 wherein said panel of genes includes at least one of MCP-1, qe82d12.x1, RACH1, PAC747L4, T-plastin, endothelin receptor type B-like, VEGF, or wx69d10.x 1.

8. A method for identifying genes which reduce an inflammatory response in a tissue comprising:
contacting a test compound with a cell-based assay system comprising an endothelial cell capable of signaling responsiveness to activated protein C (APC) by an endothelial protein C receptor (EPCR)-dependent protease activated receptor (PAR)-1 pathway;
stimulating said cell-based assay system with a test compound specific for signaling by APC or PAR1;
isolating cDNA corresponding to mRNA in stimulated cells;
analyzing the isolated cDNA using a gene expression analysis system to identify a panel of genes that are regulated in stimulated cells by signaling by APC through said EPCR-dependent PAR1 signaling pathway, wherein regulation of one or more of said genes is capable of reducing an inflammatory response in the tissue.

9. The method of claim 8 further comprising analyzing said isolated cDNA using said gene expression analysis system to identify said panel of genes that are up-regulated by signaling through PAR1 or APC and down-regulated by signaling through PAR2.

10. The method of claim 9 wherein said panel of genes includes at least one of MCP-1, qe82d12.x1, RACH1, PAC747L4, T-plastin, endothelin receptor type B-like, VEGF, or wx69d10.x1.

11. The method of claim 8 further comprising analyzing said isolated cDNA using said gene expression analysis system to identify said panel of genes that are down-regulated by signaling through PAR1 or APC and up-regulated by signaling through thrombin.

12. The method of claim 11 wherein said panel of genes includes at least one of SH-PTP3, W28170, fructose-6-phosphate, 2-kinase/fructose-2,6-bisphosphatase, W28616, BID, NF-KB2, thrombospondin-1,6-phosphofructo-2-kinase/fructose-2,6-bisphosphatase, neurofibromatosis 2 tumor suppressor, cationic amino acid transporter 2, W28616, NF-kappa-B p65delta3, TTF-I interacting peptide 12, ATRC2, alpha adducin, sortilin, p53 cellular tumor antigen, W28257, hERRa1, G protein-coupled receptor, EGFRBP-GRB2, Pax8, SH-PTP3, 5T4, bc1-xL, disintegrin-metalloprotease, C1q-related factor, cyclin D1, A1743606, myosin-IXb, GNS, ISLR, Stat2, stem cell factor, c-ets-1, usurpin-beta, chromosome 5q21-22, clone-A3-A, thrombospondin, ELL2, dual-specificity protein phosphatase, vitronectin receptor alpha subunit, PCTAIRE-2, follistatin, ets-2, ELL2, utrophin, C8FW phosphoprotein, PCTAIRE-2, fra-2, MINOR, GADD34, IL8, SSR alpha, neuron derived orphan receptor, CGGBP, nma, jun-B, epsilon tyrosine phosphatase, GRO-beta, CtIP, PRDII-BF1, vascular endothelial growth factor, stanniocalcin-related protein, CL 100, BMP-2A, NF-ATc, mrg1, jun-B, VEGF, STC, or ATF3.

13. A method for identifying a compound which modulates signaling in endothelial cells via a protease activated receptor (PAR)-1 pathway comprising:
contacting a test compound with a cell-based assay system comprising an endothelial cell capable of signaling responsiveness to activated protein C (APC) by an endothelial protein C receptor (EPCR)-dependent PAR1 pathway;
stimulating said cell-based assay system with said test compound specific for signaling by APC or PAR1;
isolating cDNA corresponding to mRNA in two populations of cells stimulated in both the presence and absence of said test compound;
analyzing said isolated cDNA using a gene expression analysis system to identify a panel of genes which are regulated in stimulated cells; and
detecting up-regulated or down-regulated genes in both populations of stimulated cells, thereby identifying said test compounds that modulate signaling through APC or PAR1.

14. The method of claim 13 wherein said detecting step further comprises identifying said test compounds that regulate gene expression by up-regulating genes that signal through PAR1 or APC and down-regulating genes that signal through PAR2.

15. The method of claim 13 wherein said detecting step further comprises identifying said test compounds that regulate gene expression by down-regulating genes that signal through PAR1 or APC and by up-regulating genes that signal through thrombin.

16. A method for detecting an activation state of an endothelial protein C receptor (EPCR)-dependent protease activated receptor (PAR)-1 pathway in a tissue comprising:
contacting a test compound with a cell-based assay system comprising an endothelial cell capable of signaling responsiveness to activated protein C (APC) by an endothelial protein C receptor (EPCR)-dependent PAR1 pathway;
stimulating said cell-based assay system with said test compound specific for signaling by APC or PAR1;
identifying a panel of genes which represent regulated genes that correlate with an activation state for endothelial cell signaling mediated by APC, EPCR or PAR1; and
determining levels of gene expression for members of said panel of genes in said tissue subject to activation mediated by APC or PAR1 thereby determining said gene activation state of said tissue.

17. The method of claim 16 wherein said determining step further comprises identifying said levels of gene expression for members of said panel of genes that are up-regulated by signaling through APC and EPCR-dependent PAR1 pathway, and identifying said panel of genes that are down-regulated by signaling through PAR2.

18. The method of claim 17 wherein said panel of genes includes at least one of MCP-1, qe82d12.x1, RACH1, PAC747L4, T-plastin, endothelin receptor type B-like, VEGF, or wx69d10.x1.

19. The method of claim 16 wherein said determining step further comprises identifying said levels of gene expression for said panel of genes that are down-regulated by signaling through APC and EPCR-dependent PAR1 pathway, and identifying said levels of gene expression for said panel of genes that are up-regulated by signaling through thrombin.

20. The method of claim 19 wherein said panel of genes includes at least one of SH-PTP3, W28170, fructose-6-phosphate, 2-kinase/fructose-2,6-bisphosphatase, W28616, BID, NF-KB2, thrombospondin-1,6-phosphofructo-2-kinase/fructose-2,6-bisphosphatase, neurofibromatosis 2 tumor suppressor, cationic amino acid transporter 2, W28616, NF-kappa-B p65delta3, TTF-I interacting peptide 12, ATRC2, alpha adducin, sortilin, p53 cellular tumor antigen, W28257, hERRa1, G protein-coupled receptor, EGFRBP-GRB2, Pax8, SH-PTP3, 5T4, bc1-xL, disintegrin-metalloprotease, C1q-related factor, cyclin D1, A1743606, myosin-IXb, GNS, ISLR, Stat2, stem cell factor, c-ets-1, usurpin-beta, chromosome 5q21-22, clone-A3-A, thrombospondin, ELL2, dual-specificity protein phosphatase, vitronectin receptor alpha subunit, PCTAIRE-2, follistatin, ets-2, ELL2, utrophin, C8FW phosphoprotein, PCTAIRE-2, fra-2, MINOR, GADD34, IL8, SSR alpha, neuron derived orphan receptor, CGGBP, nma, jun-B, epsilon tyrosine phosphatase, GRO-beta, CtIP, PRDII-BF1, vascular endothelial growth factor, stanniocalcin-related protein, CL 100, BMP-2A, NF-ATc, mrg1, jun-B, VEGF, STC, or ATF3.

21. A method for treating inflammation or sepsis in a mammalian subject comprising:
administering a therapeutically effective amount of a compound which modulates signaling in endothelial cells via a endothelial protein C receptor (EPCR)-dependent protease activated receptor (PAR)-1 pathway, wherein said compound acts as an agonist of APC-mediated PAR1 signaling in a cell-based assay system, and said compound is effective to reduce the incidence of inflammation or sepsis in the mammalian subject.

22. The method of claim 21, further comprising administering a therapeutically effective amount of a second compound acting as an antagonist of thrombin-mediated PAR1 signaling in a cell-based assay system, said second compound is effective to reduce the incidence of inflammation or sepsis in the mammalian subject.

23. The method of claim 21 further comprising identifying genes that are up-regulated by signaling through APC, EPCR, and PAR1, and identifying genes that are down-regulated by signaling through PAR2.

24. The method of claim 21 further comprising identifying genes that are down-regulated by signaling through APC, EPCR, or PAR1 and up-regulated by signaling through thrombin.

25. A method for treating stroke in a mammalian subject comprising:
administering a therapeutically effective amount of a compound which modulates signaling in endothelial cells via an endothelial protein C receptor (EPCR)-dependent protease activated receptor (PAR)-1 pathway, wherein said compound acts as an agonist of APC-mediated PAR1 signaling in a cell-based assay system and said compound is effective to reduce the incidence of stroke in the mammalian subject.

26. The method of claim 25, further comprising administering a therapeutically effective amount of a second compound acting as an antagonist of thrombin-mediated PAR1 signaling in a cell-based assay system, said second compound is effective to reduce the incidence of stroke in the mammalian subject.

27. The method of claim 25 further comprising identifying genes that are up-regulated by signaling through APC, EPCR, and PAR1, in the cell-based assay system, and identifying genes that are down-regulated by signaling through PAR2 in said cell-based assay system.

28. The method of claim 25 further comprising identifying genes that are down-regulated by signaling through APC, EPCR, or PAR1 and up-regulated by signaling through thrombin.

29. A method for treating ischemic injury in a mammalian subject comprising:
administering a therapeutically effective amount of a compound which modulates signaling in endothelial cells via a endothelial protein C receptor (EPCR)-dependent protease activated receptor (PAR)-1 pathway, wherein said compound acts as an agonist of APC-mediated PAR1 signaling in a cell-based assay system and said compound is effective to reduce the incidence of ischemic injury in the mammalian subject.

30. The method of claim 29, further comprising administering a therapeutically effective amount of a second compound acting as an antagonist of thrombin-mediated PAR1 signaling in a cell-based assay system, said second compound is effective to reduce the incidence of ischemic injury in the mammalian subject.

31. The method of claim 29 further comprising identifying genes that are up-regulated by signaling through APC, PAR1, and EPCR in said cell-based assay system, and identifying genes that are down-regulated by signaling through PAR2 in said cell-based assay system.

32. The method of claim 29 further comprising identifying genes that are down-regulated by signaling through APC, EPCR, or PAR1 and up-regulated by signaling through thrombin.

33. A method for preventing apoptosis in a mammalian cell comprising:
administering a therapeutically effective amount of a compound which modulates signaling in endothelial cells via a endothelial protein C receptor (EPCR)-dependent protease activated receptor (PAR)-1 pathway, wherein said compound acts as an agonist of PAR1 signaling in a cell-based assay system and said compound is effective to reduce the incidence of apoptosis in the mammalian cell.

34. The method of claim 33 further comprising administering a therapeutically effective amount of a second compound acting as an antagonist of thrombin-mediated PAR1 signaling in a cell-based assay system, said second compound is effective to reduce the incidence of apoptosis in the mammalian cell.

35. The method of claim 33 further comprising identifying genes that are up-regulated by signaling through APC, PAR1, and EPCR in said cell-based assay system, and identifying genes that are down-regulated by signaling through PAR2 in said cell-based assay system.

36. The method of claim 33 further comprising identifying genes that are down-regulated by signaling through APC, EPCR, or PAR1 and up-regulated by signaling through thrombin.

37. A method of identifying a test compound that prevents apoptosis in a mammalian cell comprising:
contacting a test compound to said mammalian cell in a cell-based assay system to co-express endothelial protein C receptor (EPCR) and protease activated receptor (PAR)-1 capable of signaling responsiveness to activated protein C (APC);
assaying for an effect of said test compound on death of said mammalian cell, thereby identifying compounds that prevent apoptosis in said mammalian cell.

38. A method of screening drug candidates in a mammalian subject comprising:
administering a therapeutically effective amount of a compound to said mammalian subject wherein said compound acts as an agonist of an endothelial protein C receptor (EPCR)-dependent protease activated receptor (PAR)-1 pathway, and wherein said compound modulates signaling via a PAR1 signaling pathway in an endothelial cell-based assay system.

39. The method of claim 38 further comprising administering a therapeutically effective amount of a second compound acting as an antagonist of thrombin-mediated PAR1 signaling in a cell-based assay system, said second compound modulates signaling via a PAR1 signaling pathway in an endothelial cell-based assay system.

40. The method of claim 38, wherein said signaling down-regulates genes by activated protein C (APC)/EPCR/PAR1 signaling and up-regulates genes by thrombin signaling.

41. The method of claim 40, wherein said panel of genes that are regulated in stimulated cells includes at least one of SH-PTP3, W28170, fructose-6-phosphate, 2-kinase/fructose-2,6-bisphosphatase, W28616, BID, NF-KB2, thrombospondin-1,6-phosphofructo-2-kinase/fructose-2,6-bisphosphatase, neurofibromatosis 2 tumor suppressor, cationic amino acid transporter 2, W28616, NF-kappa-B p65delta3, TTF-I interacting peptide 12, ATRC2, alpha adducin, sortilin, p53 cellular tumor antigen, W28257, hERRa1, G protein-coupled receptor, EGFRBP-GRB2, Pax8, SH-PTP3; 5T4, bc1-xL, disintegrin-metalloprotease, C1q-related factor, cyclin D1, A1743606, myosin-IXb, GNS, ISLR, Stat2, stem cell factor, c-ets-1, usurpin-beta, chromosome 5q21-22, clone-A3-A, thrombospondin, ELL2, dual-specificity protein phosphatase, vitronectin receptor alpha subunit, PCTAIRE-2, follistatin, ets-2, ELL2, utrophin, C8FW phosphoprotein, PCTAIRE-2, fra-2, MINOR, GADD34, IL8, SSR alpha, neuron derived orphan receptor, CGGBP, nma, jun-B, epsilon tyrosine phosphatase, GRO-beta, CtIP, PRDII-BF1, vascular endothelial growth factor, stanniocalcin-related protein, CL 100, BMP-2A, NF-ATc, mrg1, jun-B, VEGF, STC, or ATF3.

42. The method of claim 38, wherein said signaling up-regulates genes by APC/EPCR/PAR1 signaling and down-regulates genes by PAR2 signaling.

43. The method of claim 42, wherein said panel of genes that are regulated in stimulated cells includes at least one of MCP-1, qe82d12.x1, RACH1, PAC747L4, T-plastin, endothelin receptor type B-like, VEGF, or wx69d10.x1.

44. The method of claim 42, wherein said up-regulation by APC/EPCR/PAR1 signaling is approximately 1.3-fold or greater, and said down-regulation by PAR2 is less than approximately 1.3-fold.
